SEQUENCE LISTING (128 sequences)

SEQ ID NO: 1 - protein, 5 aa, Homo sapiens
TYGMH

SEQ ID NO: 2 - protein, 17 aa, Homo sapiens
IISPDGGHQDYADPVRG

SEQ ID NO: 3 - protein, 15 aa, Homo sapiens
TLCARTDCTWVRSDS

SEQ ID NO: 4 - protein, 12 aa, Homo sapiens
RASQRVSGDYLA

SEQ ID NO: 5 - protein, 7 aa, Homo sapiens
GASSRAT

SEQ ID NO: 6 - protein, 9 aa, Homo sapiens
QHYNGPPVT

SEQ ID NO: 7 - protein, 124 aa, Homo sapiens
QVQLEEYGGGVVQPGRSLRLSCVASGSTLGTYGMHWVRQAPGKGLEWVAIISPDGGHQDYADPVRGRFTVSRDNSKNTLYLQMDNLRVEDTAVYYCATTLCARTDCTWVRSDSWGQGTLVTVSS

SEQ ID NO: 8 - protein, 108 aa, Homo sapiens
ANVLTQSPGTLSLSPGERATLSCRASQRVSGDYLAWYQQKRGQAPRLLIYGASSRATGIPDRFSGSGSGTDFTLTISRLEPEDFAVYYCQHYNGPPVTFGQGTKLEIK

SEQ ID NO: 9 - protein, 5 aa, Homo sapiens
TFYMT

SEQ ID NO: 10 - protein, 17 aa, Homo sapiens
NIKQDGSEKDYLDSVRG

SEQ ID NO: 11 - protein, 14 aa, Homo sapiens
NGLQDSYSGDFFDH

SEQ ID NO: 12 - protein, 13 aa, Homo sapiens
SGTGSNIGHNYVS

SEQ ID NO: 13 - protein, 7 aa, Homo sapiens
DNTKRPS

SEQ ID NO: 14 - protein, 11 aa, Homo sapiens
ATWDADLSAVL

SEQ ID NO: 15 - protein, 123 aa, Homo sapiens
EVQLVESGGALVQPGGSLRLSCAASGFKFSTFYMTWIRQAPGKGLEWVANIKQDGSEKDYLDSVRGRFNISRDNAKSVLYLQMNTLSADDTGVYYCTRNGLQDSYSGDFFDHWGQGILVTVSS

SEQ ID NO: 16 - protein, 110 aa, Homo sapiens
QSVLTQPPSISAAPGQKVTISCSGTGSNIGHNYVSWYQQLPRTAPRLLIFDNTKRPSGIPDRFSGSKSGSSATLGITGLQTGDEAHYFCATWDADLSAVLFGGGTKLTVL

SEQ ID NO: 17 - protein, 5 aa, Homo sapiens
DYNMN

SEQ ID NO: 18 - protein, 17 aa, Homo sapiens
SITRSSRTIYYADSVKG

SEQ ID NO: 19 - protein, 14 aa, Homo sapiens
EDFDVSTGPYYMDV

SEQ ID NO: 20 - protein, 12 aa, Homo sapiens
RASQSVSNMFLV

SEQ ID NO: 21 - protein, 7 aa, Homo sapiens
GASTRAT

SEQ ID NO: 22 - protein, 9 aa, Homo sapiens
QQNGNSPYT

SEQ ID NO: 23 - protein, 123 aa, Homo sapiens
EVQLVESGGGLVKPGGSLRLSCAASGFIFSDYNMNWVRQAPGKGLEWVSSITRSSRTIYYADSVKGRFTISRDNAKNSLHLQMNSLRDADTAVYYCAREDFDVSTGPYYMDVWGNGTTVIVSS

SEQ ID NO: 24 - protein, 108 aa, Homo sapiens
EIVLTQSPGTLSLSPGERATLSCRASQSVSNMFLVWYQQKPGQAPRLLMYGASTRATDIPDRFSGSGSGTDFTLTISRLEPEDFAVYYCQQNGNSPYTFGQGTKLEIK

SEQ ID NO: 25 - protein, 5 aa, Homo sapiens
DYTMH

SEQ ID NO: 26 - protein, 17 aa, Homo sapiens
AISYGGTNKYYADSVKG

SEQ ID NO: 27 - protein, 9 aa, Homo sapiens
DSGYRSLLH

SEQ ID NO: 28 - protein, 16 aa, Homo sapiens
RSSQSLVHRNGYNYLD

SEQ ID NO: 29 - protein, 7 aa, Homo sapiens
MASKRAS

SEQ ID NO: 30 - protein, 8 aa, Homo sapiens
MQALQTWT

SEQ ID NO: 31 - protein, 118 aa, Homo sapiens
QVQLVESGGGVVQPGRSLRLSCAASGIAFNDYTMHWVRRSPDKGLEWVAAISYGGTNKYYADSVKGRFTISRDSSKNTLFLQMDSLRVEDTAVYYCARDSGYRSLLHWGQGTLVTVSS

SEQ ID NO: 32 - protein, 111 aa, Homo sapiens
EIVLTQSPLSLPVTPGEPASISCRSSQSLVHRNGYNYLDWYLQKPGQSPQLLIYMASKRASGVPDRFSGSGSGTEFTLKISRVEAEDVGIYYCMQALQTWTFGQGTKVEVN

SEQ ID NO: 33 - protein, 118 aa, synthetic construct
  REGION 1..118: note = 7G6_Hg
QVQLVESGGGVVQPGRSLRLSCAASGIAFNDYTMHWVRRAPGKGLEWVAAISYGGTNKYYADSVKGRFTISRDSSKNTLYLQMNSLRAEDTAVYYCARDSGYRSLLHWGQGTLVTVSS

SEQ ID NO: 34 - protein, 118 aa, synthetic construct
  REGION 1..118: note = 7G6_Hf
QVQLVESGGGVVQPGRSLRLSCAASGIAFNDYTMHWVRQAPGKGLEWVAAISYGGTNKYYADSVKGRFTISRDNSKNTLYLQMNSLRAEDTAVYYCARDSGYRSLLHWGQGTLVTVSS

SEQ ID NO: 35 - protein, 111 aa, synthetic construct
  REGION 1..111: note = 7G6_Lg
EIVLTQSPLSLPVTPGEPASISCRSSQSLVHRNGYNYLDWYLQKPGQSPQLLIYMASKRASGVPDRFSGSGSGTDFTLKISRVEAEDVGVYYCMQALQTWTFGQGTKVEIK

SEQ ID NO: 36 - protein, 111 aa, synthetic construct
  REGION 1..111: note = 7G6_Lf
DIVMTQSPLSLPVTPGEPASISCRSSQSLVHRNGYNYLDWYLQKPGQSPQLLIYMASKRASGVPDRFSGSGSGTDFTLKISRVEAEDVGVYYCMQALQTWTFGQGTKVEIK

SEQ ID NO: 37 - protein, 123 aa, synthetic construct
  REGION 1..123: note = 15E3_Hg
EVQLVESGGGLVQPGGSLRLSCAASGFKFSTFYMTWIRQAPGKGLEWVANIKQDGSEKDYLDSVRGRFTISRDNAKSSLYLQMNSLRAEDTAVYYCTRNGLQDSYSGDFFDHWGQGTLVTVSS

SEQ ID NO: 38 - protein, 123 aa, synthetic construct
  REGION 1..123: note = 15E3_Hf
EVQLVESGGGLVQPGGSLRLSCAASGFKFSTFYMTWVRQAPGKGLEWVANIKQDGSEKDYLDSVRGRFTISRDNAKNSLYLQMNSLRAEDTAVYYCTRNGLQDSYSGDFFDHWGQGTLVTVSS

SEQ ID NO: 39 - protein, 110 aa, synthetic construct
  REGION 1..110: note = 15E3_Lg
QSVLTQPPSVSAAPGQKVTISCSGTGSNIGHNYVSWYQQLPGTAPKLLIFDNTKRPSGIPDRFSGSKSGSSATLGITGLQTGDEADYFCATWDADLSAVLFGGGTKLTVL

SEQ ID NO: 40 - protein, 110 aa, synthetic construct
  REGION 1..110: note = 15E3_Lf
QSVLTQPPSVSAAPGQKVTISCSGTGSNIGHNYVSWYQQLPGTAPKLLIYDNTKRPSGIPDRFSGSKSGTSATLGITGLQTGDEADYYCATWDADLSAVLFGGGTKLTVL

SEQ ID NO: 41 - protein, 123 aa, synthetic construct
  REGION 1..123: note = 2F8_Hg
EVQLVESGGGLVQPGGSLRLSCAASGFIFSDYNMNWVRQAPGKGLEWVSSITRSSRTIYYADSVKGRFTISRDNAKNSLYLQMNSLRDEDTAVYYCAREDFDVSTGPYYMDVWGKGTTVTVSS

SEQ ID NO: 42 - protein, 108 aa, synthetic construct
  REGION 1..108: note = 2F8_Lg
EIVLTQSPGTLSLSPGERATLSCRASQSVSNMFLVWYQQKPGQAPRLLMYGASTRATGIPDRFSGSGSGTDFTLTISRLEPEDFAVYYCQQNGNSPYTFGQGTKLEIK

SEQ ID NO: 43 - protein, 108 aa, synthetic construct
  REGION 1..108: note = 2F8_Lf
EIVLTQSPGTLSLSPGERATLSCRASQSVSNMFLVWYQQKPGQAPRLLIYGASTRATGIPDRFSGSGSGTDFTLTISRLEPEDFAVYYCQQNGNSPYTFGQGTKLEIK

SEQ ID NO: 44 - protein, 124 aa, synthetic construct
  REGION 1..124: note = 17H9_Hg
QVQLVESGGGVVQPGRSLRLSCAASGSTLGTYGMHWVRQAPGKGLEWVAIISPDGGHQDYADPVRGRFTVSRDNSKNTLYLQMNSLRAEDTAVYYCATTLCARTDCTWVRSDSWGQGTLVTVSS

SEQ ID NO: 45 - protein, 124 aa, synthetic construct
  REGION 1..124: note = 17H9_Hf
QVQLVESGGGVVQPGRSLRLSCAASGSTLGTYGMHWVRQAPGKGLEWVAIISPDGGHQDYADPVRGRFTISRDNSKNTLYLQMNSLRAEDTAVYYCATTLCARTDCTWVRSDSWGQGTLVTVSS

SEQ ID NO: 46 - protein, 108 aa, synthetic construct
  REGION 1..108: note = 17H9_Lg
ANVLTQSPGTLSLSPGERATLSCRASQRVSGDYLAWYQQKPGQAPRLLIYGASSRATGIPDRFSGSGSGTDFTLTISRLEPEDFAVYYCQHYNGPPVTFGQGTKLEIK

SEQ ID NO: 47 - protein, 108 aa, synthetic construct
  REGION 1..108: note = 17H9_Lf
EIVLTQSPGTLSLSPGERATLSCRASQRVSGDYLAWYQQKPGQAPRLLIYGASSRATGIPDRFSGSGSGTDFTLTISRLEPEDFAVYYCQHYNGPPVTFGQGTKLEIK

SEQ ID NO: 48 - protein, 108 aa, synthetic construct
  REGION 1..108: note = 17H9_Lg_A1E
ENVLTQSPGTLSLSPGERATLSCRASQRVSGDYLAWYQQKPGQAPRLLIYGASSRATGIPDRFSGSGSGTDFTLTISRLEPEDFAVYYCQHYNGPPVTFGQGTKLEIK

SEQ ID NO: 49 - protein, 108 aa, synthetic construct
  REGION 1..108: note = 17H9_Lg_N2I
AIVLTQSPGTLSLSPGERATLSCRASQRVSGDYLAWYQQKPGQAPRLLIYGASSRATGIPDRFSGSGSGTDFTLTISRLEPEDFAVYYCQHYNGPPVTFGQGTKLEIK

SEQ ID NO: 50 - protein, 123 aa, synthetic construct
  REGION 1..123: note = 2F8_Hg_M115Y
EVQLVESGGGLVQPGGSLRLSCAASGFIFSDYNMNWVRQAPGKGLEWVSSITRSSRTIYYADSVKGRFTISRDNAKNSLYLQMNSLRDEDTAVYYCAREDFDVSTGPYYYDVWGKGTTVTVSS

SEQ ID NO: 51 - protein, 14 aa, synthetic construct
  REGION 1..14: note = 2F8 CDRH3 variant
EDFNVYTGPYYYDV

SEQ ID NO: 52 - protein, 7 aa, synthetic construct
  REGION 1..7: note = 7G6 CDRL2 variant
MASNRAS

SEQ ID NO: 53 - protein, 123 aa, synthetic construct
  REGION 1..123: note = 2F8 VH variant
EVQLVESGGGLVQPGGSLRLSCAASGFIFSDYNMNWVRQAPGKGLEWVSSITRSSRTIYYADSVKGRFTISRDNAKNSLYLQMNSLRDEDTAVYYCAREDFNVYTGPYYYDVWGKGTTVTVSS

SEQ ID NO: 54 - protein, 111 aa, synthetic construct
  REGION 1..111: note = 7G6 VL variant
EIVLTQSPLSLPVTPGEPASISCRSSQSLVHRNGYNYLDWYLQKPGQSPQLLIYMASNRASGVPDRFSGSGSGTDFTLKISRVEAEDVGVYYCMQALQTWTFGQGTKVEIK

SEQ ID NO: 55 - protein, 139 aa, Arachis hypogaea
RQQWELQGDRRCQSQLERANLRPCEQHLMQKIQRDEDSYGRDPYSPSQDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 56 - protein, 151 aa, Arachis hypogaea
RQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 57 - protein, 507 aa, Arachis hypogaea
RQQPEENACQFQRLNAQRPDNRIESEGGYIETWNPNNQEFECAGVALSRLVLRRNALRRPFYSNAPQEIFIQQGRGYFGLIFPGCPRHYEEPHTQGRRSQSQRPPRRLQGEDQSQQQRDSHQKVHRFDEGDLIAVPTGVAFWLYNDHDTDVVAVSLTDTNNNDNQLDQFPRRFNLAGNTEQEFLRYQQQSRQSRRRSLPYSPYSPQSQPRQEEREFSPRGQHSRRERAGQEEENEGGNIFSGFTPEFLEQAFQVDDRQIVQNLRGETESEEEGAIVTVRGGLRILSPDRKRRADEEEEYDEDEYEYDEEDRRRGRGSRGRGNGIEETICTASAKKNIGRNRSPDIYNPQAGSLKTANDLNLLILRWLGPSAEYGNLYRNALFVAHYNTNAHSIIYRLRGRAHVQVVDSNGNRVYDEELQEGHVLVVPQNFAVAGKSQSENFEYVAFKTDSRPSIANLAGENSVIDNLPEEVVANSYGLQREQARQLKNNNPFKFFVPPSQQSPRAVA

SEQ ID NO: 58 - protein, 124 aa, Arachis hypogaea
MRRERGRQGDSSSCERQVDRVNLKPCEQHIMQRIMGEQEQYDSYDIRSTRSSDQQQRCCDELNEMENTQRCMCEALQQIMENQCDRLQDRQMVQQFKRELMNLPQQCNFRAPQRCDLDVSGGRC

SEQ ID NO: 59 - protein, 330 aa, Homo sapiens [sequence omitted]

SEQ ID NO: 60 - protein, 327 aa, synthetic construct
  REGION 1..327: note = IgG4 S228P
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 107 aa, Homo sapiens [sequence omitted]

SEQ ID NO: 62 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 63 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 64 - DNA, 15 bp, Homo sapiens
  source 1..15: mol_type = unassigned DNA
acctatggcatgcac

SEQ ID NO: 65 - DNA, 51 bp, Homo sapiens
  source 1..51: mol_type = unassigned DNA
attatatcacctgatggaggtcaccaagactatgcagaccccgtgaggggc

SEQ ID NO: 66 - DNA, 45 bp, Homo sapiens
  source 1..45: mol_type = unassigned DNA
acgttgtgtgctaggaccgactgtacatgggtgaggtctgactcc

SEQ ID NO: 67 - DNA, 36 bp, Homo sapiens
  source 1..36: mol_type = unassigned DNA
agggccagtcagagagtcagtggcgactacttagcc

SEQ ID NO: 68 - DNA, 21 bp, Homo sapiens
  source 1..21: mol_type = unassigned DNA
ggtgcatccagcagggccact

SEQ ID NO: 69 - DNA, 27 bp, Homo sapiens
  source 1..27: mol_type = unassigned DNA
caacactataatggtccacccgtcact

SEQ ID NO: 70 - DNA, 372 bp, Homo sapiens
  source 1..372: mol_type = unassigned DNA
caggtgcaactggaggagtatgggggaggcgtggtccagcctgggaggtccctgagactctcctgtgtagcctctggatccaccttgggtacctatggcatgcactgggtccgtcaggctccaggcaaggggctggagtgggtggcaattatatcacctgatggaggtcaccaagactatgcagaccccgtgaggggccgattcaccgtttccagagacaattccaagaataccctttatctgcaaatggacaacctgagagttgaggacacggctgtttattattgtgcgaccacgttgtgtgctaggaccgactgtacatgggtgaggtctgactcctggggtcagggaaccctggtcaccgtctcctca

SEQ ID NO: 71 - DNA, 324 bp, Homo sapiens
  source 1..324: mol_type = unassigned DNA
gcaaatgtgttgacgcagtctccaggcaccctgtctttgtctccaggggaaagagccaccctctcctgcagggccagtcagagagtcagtggcgactacttagcctggtaccagcagaagcgtggccaggctcccaggctcctcatctatggtgcatccagcagggccactggcatcccagacaggttcagtggcagtgggtctgggacggacttcactctcaccatcagcagactggagcctgaagattttgcagtgtattactgtcaacactataatggtccacccgtcacttttggccaggggaccaagctggagatcaaa

SEQ ID NO: 72 - DNA, 15 bp, Homo sapiens
  source 1..15: mol_type = unassigned DNA
actttttatatgacg

SEQ ID NO: 73 - DNA, 51 bp, Homo sapiens
  source 1..51: mol_type = unassigned DNA
aatataaagcaggacggaagtgagaaagactatctggactctgtgcggggc

SEQ ID NO: 74 - DNA, 42 bp, Homo sapiens
  source 1..42: mol_type = unassigned DNA
aatggtctccaagattcttacagtggtgacttttttgaccac

SEQ ID NO: 75 - DNA, 39 bp, Homo sapiens
  source 1..39: mol_type = unassigned DNA
tctggaaccggatccaacattggacataattatgtctcc

SEQ ID NO: 76 - DNA, 21 bp, Homo sapiens
  source 1..21: mol_type = unassigned DNA
gacaatactaagcgaccctca

SEQ ID NO: 77 - DNA, 33 bp, Homo sapiens
  source 1..33: mol_type = unassigned DNA
gcaacgtgggatgccgacctgagtgctgtgctt

SEQ ID NO: 78 - DNA, 369 bp, Homo sapiens
  source 1..369: mol_type = unassigned DNA
gaagtgcaactggtggagtcggggggagccttggtccagccgggggggtccctgagactgtcctgtgcagcctctggattcaaatttagcactttttatatgacgtggatccgccaggctccagggaagggcctggagtgggtggccaatataaagcaggacggaagtgagaaagactatctggactctgtgcggggccgtttcaacatctccagagacaacgccaagagcgtcctgtatctgcagatgaacaccctgagcgccgatgacacgggagtctactattgtacgagaaatggtctccaagattcttacagtggtgacttttttgaccactggggccagggaatcctggtcaccgtctcctca

SEQ ID NO: 79 - DNA, 330 bp, Homo sapiens
  source 1..330: mol_type = unassigned DNA
cagtctgtattgacgcagccgccctcaatatctgcggccccaggacagaaggtcaccatctcctgctctggaaccggatccaacattggacataattatgtctcctggtaccaacaactcccaagaacagccccccgactcctcatttttgacaatactaagcgaccctcaggcattcctgaccgattctctggctctaagtctggctcgtcagccaccctgggcatcaccggactccagactggggacgaggcccattatttctgcgcaacgtgggatgccgacctgagtgctgtgcttttcggcggcgggaccaagctgaccgtcctg

SEQ ID NO: 80 - DNA, 15 bp, Homo sapiens
  source 1..15: mol_type = unassigned DNA
gattataacatgaat

SEQ ID NO: 81 - DNA, 51 bp, Homo sapiens
  source 1..51: mol_type = unassigned DNA
tccattactagaagtagtaggaccatttactacgcagactctgtgaagggc

SEQ ID NO: 82 - DNA, 42 bp, Homo sapiens
  source 1..42: mol_type = unassigned DNA
gaggatttcgatgtttcgactggcccctactacatggacgtc

SEQ ID NO: 83 - DNA, 36 bp, Homo sapiens
  source 1..36: mol_type = unassigned DNA
agggccagtcagagtgttagcaacatgttcttagtc

SEQ ID NO: 84 - DNA, 21 bp, Homo sapiens
  source 1..21: mol_type = unassigned DNA
ggtgcatctaccagggccact

SEQ ID NO: 85 - DNA, 27 bp, Homo sapiens
  source 1..27: mol_type = unassigned DNA
cagcagaatggtaactcaccatacact

SEQ ID NO: 86 - DNA, 369 bp, Homo sapiens
  source 1..369: mol_type = unassigned DNA
gaggtgcagctggtggagtctgggggaggcttggtcaagccgggggggtcgctgagactctcctgtgcagcgtctggattcatcttcagcgattataacatgaattgggtccgccaggctccagggaaggggctggagtgggtttcatccattactagaagtagtaggaccatttactacgcagactctgtgaagggccgattcaccatatccagagacaatgccaagaactcactgcatctgcaaatgaacagtctcagagacgcggacacggctgtgtattattgtgcgagagaggatttcgatgtttcgactggcccctactacatggacgtctggggcaacgggaccacggtcatcgtctcctca

SEQ ID NO: 87 - DNA, 324 bp, Homo sapiens
  source 1..324: mol_type = unassigned DNA
gaaattgtgttgacgcagtctccaggcaccctgtctttgtctccaggggaaagagccaccctctcctgcagggccagtcagagtgttagcaacatgttcttagtctggtatcagcagaaacctggccaggctcccaggctcctcatgtatggtgcatctaccagggccactgacatcccagacaggttcagtggcagtgggtctgggacagacttcactctcaccatcagcagactggagcctgaagattttgcagtgtattactgtcagcagaatggtaactcaccatacacttttggccaggggaccaagctggagatcaaa

SEQ ID NO: 88 - DNA, 42 bp, synthetic construct
  misc_feature 1..42: note = 2F8 variant CDRH3
  source 1..42: mol_type = other DNA
gaggatttcaacgtttacactggcccctactactacgacgtc

SEQ ID NO: 89 - DNA, 369 bp, synthetic construct
  misc_feature 1..369: note = 2F8 variant VH
  source 1..369: mol_type = other DNA
gaggtgcagctggtggagtctgggggaggcttggtccagccgggggggtcgctgagactctcctgtgcagcgtctggattcatcttcagcgattataacatgaattgggtccgccaggctccagggaaggggctggagtgggtttcatccattactagaagtagtaggaccatttactacgcagactctgtgaagggccgattcaccatatccagagacaatgccaagaactcactgtatctgcaaatgaacagtctcagagacgaggacacggctgtgtattattgtgcgagagaggatttcaacgtttacactggcccctactactacgacgtctggggcaaggggaccacggtcaccgtctcctca

SEQ ID NO: 90 - DNA, 15 bp, Homo sapiens
  source 1..15: mol_type = unassigned DNA
gactacactatgcac

SEQ ID NO: 91 - DNA, 51 bp, Homo sapiens
  source 1..51: mol_type = unassigned DNA
gctatatcatatggtgggactaataaatactacgcagattccgtgaagggc

SEQ ID NO: 92 - DNA, 27 bp, Homo sapiens
  source 1..27: mol_type = unassigned DNA
gattctggttatcggagtcttttgcac

SEQ ID NO: 93 - DNA, 48 bp, Homo sapiens
  source 1..48: mol_type = unassigned DNA
aggtcgagtcagagcctcgtgcatagaaatggatacaactatttagat

SEQ ID NO: 94 - DNA, 21 bp, Homo sapiens
  source 1..21: mol_type = unassigned DNA
atggcttctaaacgggcctcc

SEQ ID NO: 95 - DNA, 24 bp, Homo sapiens
  source 1..24: mol_type = unassigned DNA
atgcaagctctacaaacttggacg

SEQ ID NO: 96 - DNA, 354 bp, Homo sapiens
  source 1..354: mol_type = unassigned DNA
caggtgcagctggtggagtctgggggaggcgtggtgcagcctgggaggtccctgagactctcatgtgcagcctctggcatcgccttcaatgactacactatgcactgggtccgccggtctccagacaagggcctggagtgggtggcagctatatcatatggtgggactaataaatactacgcagattccgtgaagggccgattcaccatctccagagacagttccaagaacaccctgtttctgcagatggacagcctgagagttgaggacacggctgtgtattactgtgcgagagattctggttatcggagtcttttgcactggggccagggaaccctggtcaccgtctcctca

SEQ ID NO: 97 - DNA, 333 bp, Homo sapiens
  source 1..333: mol_type = unassigned DNA
gagattgtgttgactcagtctccactctccctgcccgtcacccctggtgagccggcctccatctcctgcaggtcgagtcagagcctcgtgcatagaaatggatacaactatttagattggtacctgcagaagccagggcagtctccacagctcctgatctatatggcttctaaacgggcctccggggtccctgacaggttcagtggcagtgggtcaggcacagaatttacactgaaaatcagcagagtggaggctgaggatgttggaatttattactgcatgcaagctctacaaacttggacgttcggccaagggaccaaggtggaagtcaac

SEQ ID NO: 98 - DNA, 21 bp, synthetic construct
  misc_feature 1..21: note = 7G6 variant CDRL2
  source 1..21: mol_type = other DNA
atggcttctaaccgggcctcc

SEQ ID NO: 99 - DNA, 333 bp, synthetic construct
  misc_feature 1..333: note = 7G6 variant VL
  source 1..333: mol_type = other DNA
gagattgtgttgactcagtctccactctccttgcccgtcacccctggtgagccggcctccatctcctgcaggtcgagtcagagcctcgtgcatagaaatggatacaactatttagattggtacctgcagaagccagggcagtctccacagctcctgatctatatggcttctaaccgggcctccggggtccctgacaggttcagtggcagtgggtcaggcacagattttacactgaaaatcagcagagtggaggctgaggatgttggagtttattactgcatgcaagctctacaaacttggacgttcggccaagggaccaaggtggaaatcaaa

SEQ ID NO: 100 - protein, 122 aa, synthetic construct
QQLKESGGGLVKPGGSLKLCCKASGFTFSDYNMNWVRQAPGKGLEWIGSITRSSRTIYYADSVKGRFTLSRDNAQSTVCLQLNSLTAADTATYFCAREDFNVYTGPYYYDVWGPGTLVTVSS

SEQ ID NO: 101 - protein, 108 aa, synthetic construct
YVMMTQTPSSVSEAVGGTVTIYCRASQSVSNMFLVWYQQKPGQPPKLLIYGASTRATGVPSRFRGSGSGTQFTLTISGMKAEDVATYYCQQNGNSPYTFGGGTEVVVK

SEQ ID NO: 102 - protein, 118 aa, synthetic construct
QEQLVESGGGLVQPEGSLTLTCKASGFDFSDYTMHWVRQAPGKGPEWIAAISYGGTNKYYADSVKGRFTISRSTSLNTVTLQMTSLTAVDTATYFCARDSGYRSLLHWGQGTLVTVSS

SEQ ID NO: 103 - protein, 111 aa, synthetic construct
AAVLTQTPSPVSAAVGGTVTIKCRSSQSLVHRNGYNYLDWYQQKPGQPPKLLIYMASKRASGVPDRFSGSGSGTQFTLTISGVQCDDAATYYCMQALQTWTFGGGTKVVVK

SEQ ID NO: 104 - protein, 111 aa, synthetic construct
AAVLTQTPSPVSAAVGGTVTIKCRSSQSLVHRNGYNYLDWYQQKPGQPPKLLIYMASKRASGVPDRFSGSGSGTQFTLTISGVQCDDAATYYCMQALQTWTLGPGTKLEIK

SEQ ID NO: 105 - protein, 111 aa, synthetic construct
AAVLTQTPSPVSAAVGGTVTIKCRSSQSLVHRNGYNYLDWYQQKPGQPPKLLIYMASNRASGVPDRFSGSGSGTQFTLTISGVQCDDAATYYCMQALQTWTFGGGTKVVVK

SEQ ID NO: 106 - protein, 123 aa, synthetic construct
QQLKESGGGLVKPGGSLKLCCKASGFTFSTYGMHWVRQAPGKGLEWIGIISPDGGHQDYADPVRGRFTLSRDNAQSTVCLQLNSLTAADTATYFCARTLCARTDCTWVRSDSWGPGTLVTVSS

SEQ ID NO: 107 - protein, 108 aa, synthetic construct
DVVMTQTPSPVSAAVGGTVTINCRASQRVSGDYLAWYQQKPGQPPKLLIYGASSRATGVPSRFSGSGSGTEFTLTISDLECADAATYYCQHYNGPPVTFGGGTEVVVK

SEQ ID NO: 108 - protein, 108 aa, synthetic construct
DVVMTQTPSPVSAAVGGTVTINCRASQRVSGDYLAWYQQKPGQPPKLLIYGASSRATGVPSRFSGSGSGTEFTLTISDLECADAATYYCQHYNGPPVTFGEETKLEIK

SEQ ID NO: 109 - protein, 123 aa, synthetic construct
QKQLVESGGGLDQPAGSLKLSCKDSGFTLSTFYMTWVHQAPGKGLEWIANIKQDGSEKDYLDSVRGRFTISSDNTQNMVDLEMNSLTAADMAIYFCARNGLQDSYSGDFFDHWGQGTLVTVSS

SEQ ID NO: 110 - protein, 110 aa, synthetic construct
QPALTQPSSVSGALGGSVTITCSGTGSNIGHNYVSWYQQLPGSVPKLLMFDNTKRPSGIPERFSGSKSGTTASLTISGLQPEDEADYYCATWDADLSAVLFGGGTKLTVL

SEQ ID NO: 111 - protein, 249 aa, Homo sapiens
EIVLTQSPATLSLSPGERATLSCRSSQSLVHRNGYNYLDWYQQKPGQAPRLLIYMASKRASGVPARFSGSGSGTDFTLTISSLEPEDSALYYCMQALQTWTFGGGTKLEIKGGGGSGGGGSGGGGSGGGGSEVQLVETGGGLVQPGGSLRLSCAASGFAFNDYTMHWVRQAPGKGLEWVSAISYGGTNKYYADSVKGRFTISRDNSKDTLYLHMNSLRAEDTAVYYCARDSGYRSLLHWGQGTLVTVSS

SEQ ID NO: 112 - protein, 249 aa, Homo sapiens
EIVLTQSPLSLPVTLGQPASISCRSSQSLVHRNGYNYLDWFHQRPGQSPRRLIYMASKRASGVPDRFSGSGSGTDFTLKISRVDAEDVGVYYCMQALQTWTFGQGTKLTVLGGGGSGGGGSGGGGSGGGGSEVQLVETGGGLVQPGGSLRLSCAASGFAFNDYTMHWVRQAPGKGLEWVSAISYGGTNKYYADSVKGRFTISRDNSKDTLYLHMNSLRAEDTAVYYCARDSGYRSLLHWGQGTLVTVSS

SEQ ID NO: 113 - protein, 249 aa, Homo sapiens
EIVLTQSPLSLPVTLGQPASISCRSSQSLVHRNGYNYLDWFHQRPGQSPRRLIYMASKRASGVPDRFSGSGSGTDFTLKISRVDAEDVGVYYCMQALQTWTFGQGTKLTVLGGGGSGGGGSGGGGSGGGGSQVQLVQSGGGLVQPGGSLRLSCAASGFAFNDYTMHWVRQAPGKGLEWVSAISYGGTNKYYADSVKGRFTISRDNGKKSLYLQMNSLIPEDTALYYCAKDSGYRSLLHWGQGTTVTVSS

SEQ ID NO: 114 - protein, 249 aa, Homo sapiens
EIVLTQSPLSLPVTLGQPASISCRSSQSLVHRNGYNYLDWFHQRPGQSPRRLIYMASKRASGVPDRFSGSGSGTDFTLKISRVDAEDVGVYYCMQALQTWTFGQGTKLTVLGGGGSGGGGSGGGGSGGGGSQVQLVQSGGGLVQPGGSLRLSCAASGFAFNDYTMHWVRQAPGKGLEWVSAISYGGTNKYYADSVKGRFTISRDNSKNTLYLQMNSLRAEDTAVYYCAADSGYRSLLHWGQGTLVTVSS

SEQ ID NO: 115 - protein, 249 aa, Homo sapiens
EIVLTQSPLSLPVTLGQPASISCRSSQSLVHRNGYNYLDWFHQRPGQSPRRLIYMASKRASGVPDRFSGSGSGTDFTLKISRVDAEDVGVYYCMQALQTWTFGQGTKLTVLGGGGSGGGGSGGGGSGGGGSEVQLVESGGGLVQPGGSLRLSCAASGFAFNDYTMHWVRQAPGKGLEWVSAISYGGTNKYYADSVKGRFTISRDNSKNTLYLQMNSLRAEDTAVYYCAKDSGYRSLLHWGQGTLVTVSS

SEQ ID NO: 116 - protein, 249 aa, Homo sapiens
EIVLTQSPATLSLSPGERATLSCRSSQSLVHRNGYNYLDWYQQKPGQAPRLLIYMASKRASGVPARFSGSGSGTDFTLTISSLEPEDSALYYCMQALQTWTFGGGTKLEIKGGGGSGGGGSGGGGSGGGGSQVQLVQSGGGLVQPGGSLRLSCAASGFAFNDYTMHWVRQAPGKGLEWVSAISYGGTNKYYADSVKGRFTISRDNSKNTLYLQMNSLRAEDTAVYYCAADSGYRSLLHWGQGTLVTVSS

SEQ ID NO: 117 - protein, 249 aa, Homo sapiens
EIVLTQSPATLSLSPGERATLSCRSSQSLVHRNGYNYLDWYQQKPGQAPRLLIYMASKRASGVPARFSGSGSGTDFTLTISSLEPEDSALYYCMQALQTWTFGGGTKLEIKGGGGSGGGGSGGGGSGGGGSEVQLVESGGGLVQPGGSLRLSCAASGFAFNDYTMHWVRQAPGKGLEWVSAISYGGTNKYYADSVKGRFTISRDNSKNTLYLQMNSLRAEDTAVYYCAKDSGYRSLLHWGQGTLVTVSS

SEQ ID NO: 118 - protein, 253 aa, Homo sapiens
QSVLTQPPSVSAAPGQKVTISCSGTGSNIGHNYVSWYQQLPGTAPKLLIYDNTKRPSGIPDRFSGSKSGTSATLGITGLQTGDEADYYCATWDADLSAVLFGTGTKLTVLGGGGSGGGGSGGGGSGGGGSQVQLVQSGGGLVQPGGSLRLSCAASGFKFSTFYMTWVRQAPGKGLEWVSNIKQDGSEKDYLDSVRGRFTISRDNSKNTLYLQMNSLRAEDTAVYYCAANGLQDSYSGDFFDHWGQGTLVTVSS

SEQ ID NO: 119 - protein, 123 aa, Homo sapiens; identical to SEQ ID NO: 53

SEQ ID NO: 120 - protein, 108 aa, Homo sapiens; identical to SEQ ID NO: 24

SEQ ID NO: 121 - protein, 118 aa, Homo sapiens; identical to SEQ ID NO: 31

SEQ ID NO: 122 - protein, 111 aa, Homo sapiens; identical to SEQ ID NO: 32

SEQ ID NO: 123 - protein, 124 aa, Homo sapiens; identical to SEQ ID NO: 7

SEQ ID NO: 124 - protein, 108 aa, Homo sapiens; identical to SEQ ID NO: 8

SEQ ID NO: 125 - protein, 123 aa, Homo sapiens; identical to SEQ ID NO: 15

SEQ ID NO: 126 - protein, 110 aa, Homo sapiens; identical to SEQ ID NO: 16

SEQ ID NO: 127 - protein, 249 aa, Homo sapiens
EIVLTQSPLSLPVTPGEPASISCRSSQSLVHRNGYNYLDWYLQKPGQSPQLLIYMASKRASGVPDRFSGSGSGTEFTLKISRVEAEDVGIYYCMQALQTWTFGQGTKVEVNGGGGSGGGGSGGGGSGGGGSQVQLVESGGGVVQPGRSLRLSCAASGIAFNDYTMHWVRRSPDKGLEWVAAISYGGTNKYYADSVKGRFTISRDSSKNTLFLQMDSLRVEDTAVYYCARDSGYRSLLHWGQGTLVTVSS

SEQ ID NO: 128 - protein, 254 aa, Homo sapiens
QSVLTQPPSISAAPGQKVTISCSGTGSNIGHNYVSWYQQLPRTAPRLLIFDNTKRPSGIPDRFSGSKSGSSATLGITGLQTGDEAHYFCATWDADLSAVLFGGGTKLTVLGGGGGSGGGGSGGGGSGGGGSEVQLVESGGALVQPGGSLRLSCAASGFKFSTFYMTWIRQAPGKGLEWVANIKQDGSEKDYLDSVRGRFNISRDNAKSVLYLQMNTLSADDTGVYYCTRNGLQDSYSGDFFDHWGQGILVTVSS

(19) United States
(12) Patent Application Publication
(10) Pub. No.: US 2025/0320282 A1
(43) Pub. Date: Oct. 16, 2025

(54) ANTI-ALLERGEN ANTIBODIES AND USES THEREOF

(71) Applicant: MABYLON AG, Schlieren (CH)

(72) Inventors: Natascha WUILLEMIN, Zürich (CH); Dimitri BIELI, Basel (CH); Niccolò PENGO, Adliswil (CH); Rahel SCHEIBLING, Winterthur (CH); Chiara ARENA, Zürich (CH); Tiziana SONATI, Zürich (CH); Alcide BARBERIS, Schindellegi (CH); Pascal GASSER, Zürich (CH)

(21) Appl. No.: 18/861,983

(22) PCT Filed: May 18, 2023
(86) PCT No.: PCT/EP2023/063444

§ 371 Date: Oct. 31, 2024

(30) Foreign Application Priority Data
May 18, 2022 (WO) PCT/EP2022/063439

(51) Int. Cl. C07K 16/16; A61K 39/00; A61K 45/06; A61P 37/08

(52) U.S. Cl. CPC C07K 16/16; A61K 45/06; A61P 37/08; A61K 2039/505; C07K 2317/24; C07K 2317/31; C07K 2317/52; C07K 2317/565; C07K 2317/567; C07K 2317/622

(57) ABSTRACT

The present invention provides antibodies binding to peanut allergens, in particular to Ara h 2 and Ara h 3 or Ara h 6. The present invention also provides compositions and kits comprising distinct antibodies binding to distinct, non-overlapping epitopes of Ara h 2 and multispecific antibodies binding to distinct, non-overlapping epitopes of Ara h 2. In addition, the present invention also relates to the use of such antibodies, compositions and kits, e.g. for preventing or treating peanut allergy.

The present invention relates to antibodies binding to peanut allergens, in particular to Ara h 2 and Ara h 3 or Ara h 6. The present invention also relates to compositions and kits comprising distinct antibodies binding to distinct, non-overlapping epitopes of Ara h 2 and to multispecific antibodies binding to distinct, non-overlapping epitopes of Ara h 2. In addition, the present invention also relates to the use of such antibodies, compositions and kits, e.g. for preventing or treating peanut allergy.

Allergies are conditions caused by hypersensitivity of the immune system. Allergen encounter results in the production of allergen-binding immunoglobulin E (IgE) antibodies, which are pre-bound on FcεRI receptors on mast cells and basophils, where they trigger the release of inflammatory compounds, such as histamine, leukotriene and lipid mediators.

Peanut allergy is one of the most severe food allergies due to its prevalence, persistency, and potential severity of allergic reaction. Allergic reactions include clinical manifestations from skin, respiratory and gastrointestinal symptoms up to severe and life-threatening reactions, such as systemic anaphylaxis. Peanut allergy is the most common cause of food-induced anaphylaxis.

Up to date, at least sixteen peanut proteins were identified as allergenic. Among these peanut allergens, Ara h 1, Ara h 2, Ara h 3 and Ara h 6 are considered to be major allergens, which means that they trigger an immunological response in more than 50% of the allergic population. In particular, Ara h 2 was reported to be the dominant peanut allergen (Hemmings, Oliver et al. Ara h 2 is the dominant peanut allergen despite similarities with Ara h 6. The Journal of allergy and clinical immunology Vol. 146 (3) (2020): 621-630.e5. doi:10.1016/j.jaci.2020.03.026). In addition, Ara h 6 emerged as common and potent peanut allergen (Blanc, F et al. (2009), Capacity of purified peanut allergens to induce degranulation in a functional in vitro assay: Ara h 2 and Ara h 6 are the most efficient elicitors. Clinical & Experimental Allergy, 39:1277-1285.). In addition, Ara h 3, which makes up 19% of the total protein in peanut extracts, is classified as a major peanut allergen because it provokes sensitization of patients with this allergy.

Despite its prevalence, up to today, there is no cure for peanut allergy other than strict avoidance of peanuts and peanut-containing foods. However, total avoidance can be complicated, in particular if no declaration of ingredients is available. While allergen immunotherapy by repeated exposure to the allergen, also known as desensitization, attempts to reduce allergic sensitivity, it was recently found that it increases rather than decreases the risk of serious allergies (Chu D K, Wood R A, French S, et al. (April 2019). “Oral immunotherapy for peanut allergy (PACE): a systematic review and meta-analysis of efficacy and safety”. The Lancet. 393 (10187): 2222-2232).

Recently, antibodies against peanut allergens emerged as promising options for treating peanut allergy. For example, WO 2018/234383 describes various human monoclonal antibodies against peanut allergens.

In view of the above, it is the object of the present invention to provide improved human-derived antibodies against peanut allergens. It is also an object of the present invention to provide a composition comprising at least three distinct potent antibodies binding to distinct, non-overlapping epitopes on the major peanut allergen Ara h 2. Furthermore, it is also an object of the present invention to provide a potent multispecific antibody binding to distinct, non-overlapping epitopes on the major peanut allergen Ara h 2.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%, for example, x±5%, or x±7%, or x±10%, or x±12%, or x±15%, or x±20%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention, prophylaxis, attenuation, amelioration and therapy. The terms “subject” or “patient” are used interchangeably herein to mean all mammals including humans. Examples of subjects include humans, cows, dogs, cats, horses, goats, sheep, pigs, and rabbits. In some embodiments, the subject or patient is a human.

Doses are often expressed in relation to the bodyweight. Thus, a dose which is expressed as [g, mg, or other unit]/kg (or g, mg etc.) usually refers to [g, mg, or other unit] “per kg (or g, mg etc.) bodyweight”, even if the term “bodyweight” is not explicitly mentioned.

The term “binding” and similar reference usually means “specifically binding”, which does not encompass non-specific sticking. In particular, specific binding of an antibody means that the antibody recognizes its target antigen and binds its target with greater affinity (or at lower antibody concentrations, e.g. EC50) than it does to a structurally different antigen and/or to an antigen with a modified or mutated sequence. Thereby, a “greater” affinity may be at least 2 fold, 3 fold, 4 fold, 5 fold, 10 fold, 15 fold, 20 fold, 25 fold, 50 fold, 75 fold, 100 fold 150 fold, 200 fold, 500 fold, 750 fold, 1,000 fold, 1,500 fold, 2,000 fold, 5,000 fold, 7,500 fold, 10,000 fold or even higher affinity as compared to the binding to a control antigen. In some instances, antibody-binding to the control antigen may be undetectable (below detection threshold), while antibody-binding to the specific antigen may be well detected/determined.

As used herein, the term “antibody” encompasses various forms of antibodies including without being limited to, whole antibodies, antibody fragments (such as antigen binding fragments), human antibodies, chimeric antibodies, humanized antibodies, recombinant antibodies and genetically engineered antibodies (e.g., variant or mutant antibodies) as long as the characteristic properties according to the invention are retained. In some embodiments, the antibody is a human antibody. In some embodiments, the antibody is a monoclonal antibody. For example, the antibody may be a human monoclonal antibody.

As described above, the term “antibody” generally also includes antibody fragments. Fragments of the antibodies may retain the antigen-binding activity of the antibodies. Such fragments are referred to as “antigen-binding fragments”. Antigen-binding fragments include, but are not limited to, single chain antibodies, Fab, Fab′, F(ab′)2, Fv or scFv. Fragments of the antibodies can be obtained from the antibodies by methods that include digestion with enzymes, such as pepsin or papain, and/or by cleavage of disulfide bonds by chemical reduction. Alternatively, fragments of the antibodies can be obtained by recombinant means, for example by cloning and expressing a part (fragment) of the sequences of the heavy and/or light chain. The invention also encompasses single-chain Fv fragments (scFv) derived from the heavy and light chains of an antibody of the invention. For example, the invention includes a scFv comprising the CDRs from an antibody of the invention. Also included are heavy or light chain monomers and dimers, single domain heavy chain antibodies, single domain light chain antibodies, as well as single chain antibodies, e.g., single chain Fv in which the heavy and light chain variable domains are joined by a peptide linker. Antibody fragments of the invention may be contained in a variety of structures known to the person skilled in the art. In addition, the sequences of the invention may be a component of multispecific molecules in which the sequences of the invention target the epitopes of the invention and other regions of the molecule bind to other targets. Although the specification, including the claims, may, in some places, refer explicitly to antigen binding fragment(s), antibody fragment(s), variant(s) and/or derivative(s) of antibodies, it is understood that the term “antibody” includes all categories of antibodies, namely, antigen binding fragment(s), antibody fragment(s), variant(s) and derivative(s) of antibodies.

Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice or chicken) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). As used herein, the expression “human antibodies” includes non-naturally occurring sequence variants of human antibodies, which are usually obtained by introducing one or more mutations in the (naturally occurring) human antibodies. Such mutations include one or more mutations in a CDR or in a framework region, as well as Fc modifications (e.g., as known in the art for specific functionalities).

As used herein, the term “variable region” (variable region of a light chain (Vi), variable region of a heavy chain (VA)) denotes each of the pair of light and heavy chains which is involved directly in binding the antibody to the antigen.

Antibodies of the invention can be of any isotype (e.g., IgA, IgG, IgM i.e. an α, γ or μ heavy chain). Preferably, the antibody is of the IgG type or the IgA type. Within the IgG isotype, antibodies may be IgG1, IgG2, IgG3 or IgG4 subclass, preferably IgG1 or IgG4. Antibodies of the invention may have a κ or a λ light chain.

Antibodies according to the present invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies according to the present invention may be immunogenic in human and/or in non-human (or heterologous) hosts e.g., in mice. For example, the antibodies may have an idiotope that is immunogenic in non-human hosts, but not in a human host. Antibodies of the invention for human use include those that cannot be easily isolated from hosts such as mice, goats, rabbits, rats, non-primate mammals, etc. and cannot generally be obtained by humanization or from xeno-mice.

As used herein, the term “antigen” refers to any structural substance which serves as a target for the receptors of an adaptive immune response, in particular as a target for antibodies, T cell receptors, and/or B cell receptors. An “epitope”, also known as “antigenic determinant”, is the part (or fragment) of an antigen that is recognized by the immune system, in particular by antibodies, T cell receptors, and/or B cell receptors. Thus, one antigen has at least one epitope, i.e. a single antigen has one or more epitopes. An antigen may be (i) a peptide, a polypeptide, or a protein, (ii) a polysaccharide, (iii) a lipid, (iv) a lipoprotein or a lipopeptide, (v) a glycolipid, (vi) a nucleic acid, or (vii) a small molecule drug or a toxin. Thus, an antigen may be a peptide, a protein, a polysaccharide, a lipid, a combination thereof including lipoproteins and glycolipids, a nucleic acid (e.g. DNA, siRNA, shRNA, antisense oligonucleotides, decoy DNA, plasmid), or a small molecule drug (e.g. cyclosporine A, paclitaxel, doxorubicin, methotrexate, 5-aminolevulinic acid), or any combination thereof. Preferably, the antigen is selected from (i) a peptide, a polypeptide, or a protein, (ii) a polysaccharide, (iii) a lipid, (iv) a lipoprotein or a lipopeptide and (v) a glycolipid; more preferably, the antigen is a peptide, a polypeptide, or a protein.

As used herein, the term “mutation” relates to a change in the nucleic acid sequence and/or in the amino acid sequence in comparison to a reference sequence, e.g. a corresponding genomic sequence. A mutation, e.g. in comparison to a genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

As used herein (i.e. throughout the present specification), the term “sequence variant” refers to any alteration in comparison to a reference sequence. The term “sequence variant” includes nucleotide sequence variants and amino acid sequence variants. Preferably, a reference sequence is any of the sequences listed in the “Table of Sequences and SEQ ID Numbers” (Sequence listing), i.e. SEQ ID NO: 1 to SEQ ID NO: 99. In particular, a sequence variant shares (over the whole length of the sequence) at least 70% or at least 75%, preferably at least 80% or at least 85%, more preferably at least 90% or at least 93%, even more preferably at least 95% or at least 96%, still more preferably at least 97% or at least 98%, particularly preferably at least 99% sequence identity with its reference sequence. In some embodiments, the sequence variant shares at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity. Thereby, the higher the %-identity of a sequence variant, the more it is preferred. For example, a sequence variant having at least 84% sequence identity with a reference sequence is more preferred than a sequence variant having at least 75% sequence identity, but less than 84% sequence identity, with a reference sequence. In some embodiments, the sequence variant maintains the (biological) function of the reference sequence. For example, sequence variants relating to antibodies of the invention preferably maintain the specific binding to the peanut allergen, in particular Ara h 2 (and, optionally, additionally to Ara h 3 or Ara h 6).

Sequence identity may be calculated as described below. Usually a sequence variant may preserve the specific function of the reference sequence. In some embodiments, an amino acid sequence variant has an altered sequence in which one or more (e.g., 1, 2, 3, 4, 5, 6, 7, 8, 9, 10 or more) of the amino acids in the reference sequence is deleted or substituted, or one or more (e.g., 1, 2, 3, 4, 5, 6, 7, 8, 9, 10 or more) amino acids are inserted into or added to the sequence of the reference amino acid sequence. As a result of the alterations, the amino acid sequence variant has an amino acid sequence which is at least 70% or at least 75%, preferably at least 80% or at least 85%, more preferably at least 90% or at least 93%, even more preferably at least 95% or at least 96%, still more preferably at least 97% or at least 98%, particularly preferably at least 99% identical to the reference sequence. For example, variant sequences which are at least 90% identical have no more than 10 alterations, i.e., any combination of deletions, insertions or substitutions, per 100 amino acids of the reference sequence. The same, of course, also applies similarly to nucleic acid sequences.

The “% identity” of the sequence variant is usually determined with respect to the reference sequence. It is usually calculated with regard to the full length of the reference sequence (i.e. the sequence recited in the application). Percentage identity, as referred to herein, can be determined, for example, by methods known in the art, such as BLAST using the default parameters specified by the NCBI (the National Center for Biotechnology Information; http://www.ncbi.nlm.nih.gov/) [Blosum 62 matrix; gap open penalty=11 and gap extension penalty=1].

In general, while it is possible to have non-conservative amino acid substitutions, the substitutions are preferably conservative amino acid substitutions, wherein the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydroxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, cysteine, and glycine, with another.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

### Antibodies and Antigen-Binding Fragments Thereof

In a first aspect the present invention provides an (isolated) antibody, or an antigen-binding fragment thereof, which (specifically) binds to a peanut allergen, in particular to Ara h 2 (Arachis hypogaea allergen 2). Ara h 2 is a major peanut allergen, which is recognized by serum IgE from more than 90% of patients with peanut hypersensitivity. Ara h 2 is a 2S albumin storage protein of approximately 17.5 kDa. Two Ara h 2 isoforms are described, namely, Ara h 2.0101 (SEQ ID NO: 55) and Ara h 2.0201 (SEQ ID NO: 56) with Ara h 2.0201 containing twelve additional amino acids (Hales, Belinda et al. (2004). Isoforms of the Major Peanut Allergen Ara h 2: IgE Binding in Children with Peanut Allergy. International archives of allergy and immunology. 135. 101-7. 10.1159/000080652). Accordingly, the antibody, or the antigen-binding fragment thereof, of the present invention binds in particular to a polypeptide or protein having an amino acid sequence according to SEQ ID NO: 55 and/or 56.

Preferably, the (isolated) antibody, or an antigen-binding fragment thereof, which (specifically) binds to Ara h 2, further binds (specifically) to Ara h 3 (Arachis hypogaea allergen 3; also referred to as “Ara h 3.0101”; SEQ ID NO: 57) or to Ara h 6 (Arachis hypogaea allergen 6; also referred to as “Ara h 6.0101”; SEQ ID NO: 58). Ara h 3 and Ara h 6 are further major peanut allergens. Accordingly, the antibody, or the antigen-binding fragment thereof, of the present invention binds preferably to a polypeptide or protein having an amino acid sequence according to SEQ ID NO: 57 or 58. Preferably, the antibody, or the antigen-binding fragment thereof, of the invention binds (specifically) (i) to Ara h 2 and Ara h 3; or (ii) to Ara h 2 and Ara h 6.

The peanut allergen, in particular Ara h 2, Ara h 3 and/or Ara h 6, may be of peanut origin, recombinantly expressed or a synthetic peanut peptide.

Standard methods to assess binding of the antibody according to the present invention, or the antigen-binding fragment thereof, are known to those skilled in the art and include, for example, ELISA (enzyme-linked immunosorbent assay). Thereby, the relative affinities of antibody binding may be determined by measuring the concentration of the antibody (EC50) required to achieve 50% maximal binding at saturation. A specific example of an ELISA, which may be used to assess binding of an antibody, is described in the example section of this specification.

In general, the antibody, or an antigen-binding fragment thereof, according to the present invention, may comprise (at least) three complementarity determining regions (CDRs) on a heavy chain and (at least) three CDRs on a light chain. In general, complementarity determining regions (CDRs) are the hypervariable regions present in heavy chain variable domains and light chain variable domains. Typically, the CDRs of a heavy chain and the connected light chain of an antibody together form the antigen receptor. Usually, the three CDRs (CDR1, CDR2, and CDR3) are arranged non-consecutively in the variable domain. Since antigen receptors are typically composed of two variable domains (on two different polypeptide chains, i.e. heavy and light chain: heavy chain variable region (VH) and light chain variable region (VL)), there are typically six CDRs for each antigen receptor (heavy chain: CDRH1, CDRH2, and CDRH3; light chain: CDRL1, CDRL2, and CDRL3). For example, a classical IgG antibody molecule usually has two antigen receptors and therefore contains twelve CDRs. The CDRs on the heavy and/or light chain may be separated by framework regions, whereby a framework region (FR) is a region in the variable domain which is less “variable” than the CDR. For example, a variable region (or each variable region, respectively) may be composed of four framework regions, separated by three CDR's.

The sequences of the heavy chains and light chains of exemplary antibodies of the invention, comprising three different CDRs on the heavy chain and three different CDRs on the light chain were determined. The CDR amino acid sequences of the CDR1 of the heavy chain (CDRH1), the CDR2 of the heavy chain (CDRH2), the CDR3 of the heavy chain (CDRH3), the CDR1 of the light chain (CDRL1), the CDR2 of the light chain (CDRL2) and the CDR3 of the light chain (CDRL3) of exemplary antibodies 17H9, 15E3, 2F8 and 7G6, and exemplary variants thereof, are shown in Table 1 below.

The CDRs, in particular the three different CDRs (CDR1, CDR2 and CDR3) on the heavy chain and three different CDRs (CDR1, CDR2 and CDR3) on the light chain, as identified by the present invention may be grafted on any variable framework region, in particular any variable human framework region, without abrogating their specificity.

The human variable framework regions of the heavy chain (VH) may be retrieved from the website: “https://www.imgt.org/genedb/resultPage.action;jsessionid=49EB34C22C79EAC51862C761 08963216?gene.id.species=Homo+sapiens&molComponent=IG&geneTypeLike=variable&a llele.fcode=functional&cloneName=&locusLike=IGH&mainLocusLike=IGH+locus&cosLoc usLike=any&groupLike=any&subgroup=−1&geneLike=&selection=any”, the contents of which is incorporated herein by reference. Thus, the VH chain may be selected from the group consisting of the amino acid sequences encoded by the genes IGHV1-18, IGHV1-2, IGHV1-24, IGHV1-3, IGHV1-45, IGHV1-46, IGHV1-58, IGHV1-69, IGHV1-69-2, IGHV1-69D, IGHV1-8, IGHV2-26, IGHV2-5, IGHV2-70, IGHV2-70D, IGHV3-11, IGHV3-13, IGHV3-15, IGHV3-20, IGHV3-21, IGHV3-23, IGHV3-23D, IGHV3-30, IGHV3-30-3, IGHV3-30-5, IGHV3-33, IGHV3-35, IGHV3-43, IGHV3-43D, IGHV3-48, IGHV3-49, IGHV3-53, IGHV3-62, IGHV3-64, IGHV3-64D, IGHV3-66, IGHV3-7, IGHV3-72, IGHV3-73, IGHV3-74, IGHV3-9, IGHV3-NL1, IGHV4-28, IGHV4-30-1, IGHV4-30-2, IGHV4-30-4, IGHV4-31, IGHV4-34, and IGHV4-38-2.

The human variable framework regions of the light heavy chain (VK, kappa) may be retrieved from the website: “https://www.imgt.org/genedb/resultPage.action;jsessionid=49EB34C22C79EAC51862C761 08963216?gene.id.species=Homo+sapiens&molComponent=IG&geneTypeLike=variable&a llele.fcode-functional&cloneName=&locusLike=IGK&mainLocusLike=IGK+locus&cosLocu sLike=any&groupLike=any&subgroup=−1&geneLike=&selection=any”, the contents of which is incorporated herein by reference. Thus, the VK (kappa) chain may be selected from the group consisting of the amino acid sequences encoded by the genes IGKV1-12, IGKV1-13, IGKV1-16, IGKV1-17, IGKV1-27, IGKV1-33, IGKV1-39, IGKV1-5, IGKV1-6, IGKV1-8, IGKV1-9, IGKV1-NL1, IGKV1D-12, IGKV1D-13, IGKVID-16, IGKV1D-17, IGKV1D-33, IGKV1D-39, IGKV1D-43, IGKV1D-8, IGKV2-24, IGKV2-28, IGKV2-29, IGKV2-30, IGKV2-40, IGKV2D-26, IGKV2D-28, IGKV2D-29, IGKV2D-30, IGKV2D-40, IGKV3-11, IGKV3-15, IGKV3-20, IGKV3D-11, IGKV3D-15, IGKV3D-20, IGKV3D-7, IGKV4-1, IGKV5-2, IGKV6-21, and IGKV6D-21.

The human variable framework regions of the light heavy chain (VL, lambda) may be retrieved from the website: “https://www.imgt.org/genedb/resultPage.action;jsessionid=49EB34C22C79EAC51862C761 08963216?gene.id.species=Homo+sapiens&molComponent=IG&geneTypeLike=variable&a llele.fcode=functional&cloneName=&locusLike=IGL&mainLocusLike=IGL+locus&cosLocus Like=any&groupLike=any&subgroup=−1&geneLike=&selection=any”, the contents of which incorporated herein by reference. Thus, the VL (lambda) chain may be selected from the group consisting of the amino acid sequences encoded by the genes IGLV1-36, IGLV1-40, IGLV1-44, IGLV1-47, IGLV1-51, IGLV10-54, IGLV2-11, IGLV2-14, IGLV2-18, IGLV2-23, IGLV2-8, IGLV3-1, IGLV3-10, IGLV3-12, IGLV3-16, IGLV3-19, IGLV3-21, IGLV3-22, IGLV3-25, IGLV3-27, IGLV3-9, IGLV4-3, IGLV4-60, IGLV4-69, IGLV5-37, IGLV5-39, IGLV5-45, IGLV5-52, IGLV6-57, IGLV7-43, IGLV7-46, IGLV8-61, and IGLV9-49.

The human variable framework region of the heavy and the light chain may also contain the respective human HJ (heavy chain), and light chain KJ (kappa) or LJ (lambda) sequences.

The HJ sequences may be retrieved from the website: “https://www.imgt.org/genedb/resultPage.action?gene.id.species=Homo+sapiens&molComp onent=IG&geneTypeLike=any&allele.fcode=functional&cloneName=&locusLike=IGH&mai nLocusLike=IGH+locus&cosLocusLike=any&groupLike=IGHJ&subgroup=-1&geneLike=&selection=any”, the contents of which is incorporated herein by reference. In particular, the HJ sequence may be selected from amino acid sequences encoded by the genes consisting of the group: IGHJ1, IGHJ2, IGHJ3, IGHJ4, IGHJ5, and IGHJ6.

The KJ sequences may be retrieved from the website: “https://www.imgt.org/genedb/resultPage.action?gene.id.species=Homo+sapiens&molComp onent=IG&geneTypeLike=any&allele.fcode=functional&cloneName=&locusLike=IGK&mai nLocusLike=IGK+locus&cosLocusLike=any&groupLike=IGKJ&subgroup=-1 &geneLike=&selection=any”, the contents of which is incorporated herein by reference. In particular, the KJ sequence may be selected from amino acid sequences encoded by the genes consisting of the group IGKJ1, IGKJ2, IGKJ3, IGKJ4, and IGKJ5.

The LJ sequences may be retrieved from the website: “https://www.imgt.org/genedb/resultPage.action?gene.id.species=Homo+sapiens&molComp onent=IG&geneTypeLike=any&allele.fcode-functional&cloneName=&locusLike=IGL&mai nLocusLike=IGL+locus&cosLocusLike=any&groupLike=IGLJ&subgroup=-1&geneLike=&selection=any”, the contents of which is incorporated herein by reference. In particular, the LJ sequence may be selected from amino acid sequences encoded by the genes consisting of the group: IGLJ1, IGLJ2, IGLJ3, IGLJ6, and IGLJ7.

The heavy chain CDR sequences of the invention (CDRH1, CDRH2 and CDRH3) may be comprised by a human heavy chain variable framework sequence as defined by a human VH sequence as described herein in combination with a human HJ sequence as described herein. Analogously, the light chain CDR sequences of the invention (CDRL1, CDRL2 and CDRL3) may be comprised by a human light chain variable framework sequence as defined by a human VL or VK sequence as described herein in combination with a human LJ or KJ sequence as described.

The combination of (i) a human VH sequence (comprising an CDRH1, an CDRH2 and an CDRH3 sequence, respectively, according to the invention) and of a human VL sequence (comprising an CDRL1, an CDRL2 and an CDRL3 sequence, respectively, of the invention) or of (ii) a human VH sequence (comprising an CDRH1, an CDRH2 and an CDRH3 sequence, respectively, of the invention) and of a human VK sequence (comprising an CDRL1, an CDRL2 and an CDRL3 sequence, respectively, of the invention) may thus characterize the variable framework region of an antibody according to the invention and its binding region. Also, the variable framework region may be characterized (i) by a human VH sequence and a human HJ sequence in combination with a human VL and a human LJ sequence or (ii) by a human VH sequence and a human HJ sequence in combination with a human VK and a human KJ sequence, thereby forming the variable framework region of an antibody according to the invention and its binding region.

The CDR sequences of the invention are inserted at the sites of the variable framework sequences which present the CDR sequences, e.g. as indicated by grey boxes in FIGS. 18B, 19D, 20C and 21B.

The numbering of the residues in the variable regions was done according to the IMGT numbering system (IMGT: http://www.imgt.org/; cf. Lefranc, M.-P. et al. (2009) Nucleic Acids Res. 37, D1006-D1012). To define the CDR regions, the Kabat CDR definition was applied (Tai Te Wu, Elvin A. Kabat; An analysis of the sequences of the variable regions of Bence Jones proteins and myeloma light chains and their implications for antibody complementarity. J Exp Med 1 Aug. 1970; 132 (2): 211-250; George Johnson, Tai Te Wu, Kabat Database and its applications: 30 years after the first variability plot, Nucleic Acids Research, Volume 28, Issue 1, 1 Jan. 2000, Pages 214-218).

TABLE 1
SEQ ID NOs for CDR sequences of antibodies
17H9, 15E3, 2F8 and 7G6

| Antibody | CDRH1 | CDRH2 | CDRH3 | CDRL1 | CDRL2 | CDRL3 |
|---|---|---|---|---|---|---|
| 17H9 | 1 | 2 | 3 | 4 | 5 | 6 |
| 15E3 | 9 | 10 | 11 | 12 | 13 | 14 |
| 2F8 | 17 | 18 | 19, 51 | 20 | 21 | 22 |
| 7G6 | 25 | 26 | 27 | 28 | 29, 52 | 30 |

Furthermore, the amino acid sequences of the variable regions of the heavy chain (VH) and the light chain (VL) of exemplary antibodies 17H9, 15E3, 2F8 and 7G6, and exemplary variants thereof, are shown in Table 2 below:

TABLE 2
SEQ ID NOs for wild-type (wt) and engineered VH/VL
sequences of antibodies 17H9, 15E3, 2F8 and 7G6

| | VH | | | | VL | | | |
|---|---|---|---|---|---|---|---|---|
| | | engineered | | | | engineered | | |
| Antibody | wt | Hg | Hf | add. VH | wt | Lg | Lf | add. VL |
| 17H9 | 7 | 44 | 45 | — | 8 | 46 | 47 | 48, 49 |
| 15E3 | 15 | 37 | 38 | — | 16 | 39 | 40 | — |
| 2F8 | 23 | 41 | — | 50, 53 | 24 | 42 | 43 | — |
| 7G6 | 31 | 33 | 34 | — | 32 | 35 | 36 | 54 |

Preferably, the antibody of the invention, or the antigen-binding fragment thereof, comprises the combination of six CDR sequences of the exemplified antibodies shown in Table 1 (optionally the VH and VL sequences of the exemplified antibodies shown in Table 2), or sequence variants thereof, as defined herein.

In some embodiments, the antibody, or the antigen-binding fragment thereof, comprises a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6. Preferably, the antibody or the antigen-binding fragment thereof comprises:
a heavy chain CDR1 sequence according to SEQ ID NO: 1;
a heavy chain CDR2 sequence according to SEQ ID NO: 2;
a heavy chain CDR3 sequence according to SEQ ID NO: 3;
a light chain CDR1 sequence according to SEQ ID NO: 4;
a light chain CDR2 sequence according to SEQ ID NO: 5; and
a light chain CDR3 sequence according to SEQ ID NO: 6.

As shown in the appended examples, such an antibody (e.g., 17H9) binds specifically to Ara h 2 and Ara h 3.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 7 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 8. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 7 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 46. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 7 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 47. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 7 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 48. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 7 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 49. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 44 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 8. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 44 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 46. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 44 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 47. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 44 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 48. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 44 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 49. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 45 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 8. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 45 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 46. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 45 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 47. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 45 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 48. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

Preferably, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 45 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 49. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, the antibody, or an antigen-binding fragment thereof, comprises a heavy chain variable region comprising or consisting of an amino acid sequence as set forth in SEQ ID NO: 7 and a light chain variable region comprising or consisting of an amino acid sequence as set forth in SEQ ID NO: 8.

In some embodiments, the antibody, or an antigen-binding fragment thereof, comprises a heavy chain variable region comprising or consisting of an amino acid sequence as set forth in SEQ ID NO: 7 and a light chain variable region comprising or consisting of an amino acid sequence as set forth in SEQ ID NO: 46.

In some embodiments, the antibody, or an antigen-binding fragment thereof, comprises a heavy chain variable region comprising or consisting of an amino acid sequence as set forth in SEQ ID NO: 7 and a light chain variable region comprising or consisting of an amino acid sequence as set forth in SEQ ID NO: 47.

In some embodiments, the antibody, or an antigen-binding fragment thereof, comprises a heavy chain variable region comprising or consisting of an amino acid sequence as set forth in SEQ ID NO: 7 and a light chain variable region comprising or consisting of an amino acid sequence as set forth in SEQ ID NO: 48.

In some embodiments, the antibody, or an antigen-binding fragment thereof, comprises a heavy chain variable region comprising or consisting of an amino acid sequence as set forth in SEQ ID NO: 7 and a light chain variable region comprising or consisting of an amino acid sequence as set forth in SEQ ID NO: 49.

In some embodiments, the antibody, or an antigen-binding fragment thereof, comprises a heavy chain variable region comprising or consisting of an amino acid sequence as set forth in SEQ ID NO: 44 and a light chain variable region comprising or consisting of an amino acid sequence as set forth in SEQ ID NO: 8.

In some embodiments, the antibody, or an antigen-binding fragment thereof, comprises a heavy chain variable region comprising or consisting of an amino acid sequence as set forth in SEQ ID NO: 44 and a light chain variable region comprising or consisting of an amino acid sequence as set forth in SEQ ID NO: 46.

In some embodiments, the antibody, or an antigen-binding fragment thereof, comprises a heavy chain variable region comprising or consisting of an amino acid sequence as set forth in SEQ ID NO: 44 and a light chain variable region comprising or consisting of an amino acid sequence as set forth in SEQ ID NO: 47.

In some embodiments, the antibody, or an antigen-binding fragment thereof, comprises a heavy chain variable region comprising or consisting of an amino acid sequence as set forth in SEQ ID NO: 44 and a light chain variable region comprising or consisting of an amino acid sequence as set forth in SEQ ID NO: 48.

In some embodiments, the antibody, or an antigen-binding fragment thereof, comprises a heavy chain variable region comprising or consisting of an amino acid sequence as set forth in SEQ ID NO: 44 and a light chain variable region comprising or consisting of an amino acid sequence as set forth in SEQ ID NO: 49.

In some embodiments, the antibody, or an antigen-binding fragment thereof, comprises a heavy chain variable region comprising or consisting of an amino acid sequence as set forth in SEQ ID NO: 45 and a light chain variable region comprising or consisting of an amino acid sequence as set forth in SEQ ID NO: 8.

In some embodiments, the antibody, or an antigen-binding fragment thereof, comprises a heavy chain variable region comprising or consisting of an amino acid sequence as set forth in SEQ ID NO: 45 and a light chain variable region comprising or consisting of an amino acid sequence as set forth in SEQ ID NO: 46.

In some embodiments, the antibody, or an antigen-binding fragment thereof, comprises a heavy chain variable region comprising or consisting of an amino acid sequence as set forth in SEQ ID NO: 45 and a light chain variable region comprising or consisting of an amino acid sequence as set forth in SEQ ID NO: 47.

In some embodiments, the antibody, or an antigen-binding fragment thereof, comprises a heavy chain variable region comprising or consisting of an amino acid sequence as set forth in SEQ ID NO: 45 and a light chain variable region comprising or consisting of an amino acid sequence as set forth in SEQ ID NO: 48.

Preferably, the antibody, or an antigen-binding fragment thereof, comprises a heavy chain variable region comprising or consisting of an amino acid sequence as set forth in SEQ ID NO: 45 and a light chain variable region comprising or consisting of an amino acid sequence as set forth in SEQ ID NO: 49.

In some embodiments, the antibody, or the antigen-binding fragment thereof, comprises a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14. Preferably, the antibody or the antigen-binding fragment thereof comprises:
a heavy chain CDR1 sequence according to SEQ ID NO: 9;
a heavy chain CDR2 sequence according to SEQ ID NO: 10;
a heavy chain CDR3 sequence according to SEQ ID NO: 11;
a light chain CDR1 sequence according to SEQ ID NO: 12;
a light chain CDR2 sequence according to SEQ ID NO: 13; and
a light chain CDR3 sequence according to SEQ ID NO: 14.

As shown in the appended examples, such an antibody (e.g., 15E3) binds specifically to Ara h 2 and Ara h 6.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 15 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 16. Thereby, the CDR sequences as defined above (heavy chain CDR1, CDR2, and CDR3 sequences as set forth in SEQ ID NO: 9, SEQ ID NO: 10, and SEQ ID NO: 11, respectively; and light chain CDR1, CDR2, and CDR3 sequences as set forth in SEQ ID NO: 12, SEQ ID NO: 13, and SEQ ID NO: 14, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 15 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 39. Thereby, the CDR sequences as defined above (heavy chain CDR1, CDR2, and CDR3 sequences as set forth in SEQ ID NO: 9, SEQ ID NO: 10, and SEQ ID NO: 11, respectively; and light chain CDR1, CDR2, and CDR3 sequences as set forth in SEQ ID NO: 12, SEQ ID NO: 13, and SEQ ID NO: 14, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 15 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 40. Thereby, the CDR sequences as defined above (heavy chain CDR1, CDR2, and CDR3 sequences as set forth in SEQ ID NO: 9, SEQ ID NO: 10, and SEQ ID NO: 11, respectively; and light chain CDR1, CDR2, and CDR3 sequences as set forth in SEQ ID NO: 12, SEQ ID NO: 13, and SEQ ID NO: 14, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 37 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 16. Thereby, the CDR sequences as defined above (heavy chain CDR1, CDR2, and CDR3 sequences as set forth in SEQ ID NO: 9, SEQ ID NO: 10, and SEQ ID NO: 11, respectively; and light chain CDR1, CDR2, and CDR3 sequences as set forth in SEQ ID NO: 12, SEQ ID NO: 13, and SEQ ID NO: 14, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 37 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 39. Thereby, the CDR sequences as defined above (heavy chain CDR1, CDR2, and CDR3 sequences as set forth in SEQ ID NO: 9, SEQ ID NO: 10, and SEQ ID NO: 11, respectively; and light chain CDR1, CDR2, and CDR3 sequences as set forth in SEQ ID NO: 12, SEQ ID NO: 13, and SEQ ID NO: 14, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 37 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 40. Thereby, the CDR sequences as defined above (heavy chain CDR1, CDR2, and CDR3 sequences as set forth in SEQ ID NO: 9, SEQ ID NO: 10, and SEQ ID NO: 11, respectively; and light chain CDR1, CDR2, and CDR3 sequences as set forth in SEQ ID NO: 12, SEQ ID NO: 13, and SEQ ID NO: 14, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 38 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 16. Thereby, the CDR sequences as defined above (heavy chain CDR1, CDR2, and CDR3 sequences as set forth in SEQ ID NO: 9, SEQ ID NO: 10, and SEQ ID NO: 11, respectively; and light chain CDR1, CDR2, and CDR3 sequences as set forth in SEQ ID NO: 12, SEQ ID NO: 13, and SEQ ID NO: 14, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 38 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 40. Thereby, the CDR sequences as defined above (heavy chain CDR1, CDR2, and CDR3 sequences as set forth in SEQ ID NO: 9, SEQ ID NO: 10, and SEQ ID NO: 11, respectively; and light chain CDR1, CDR2, and CDR3 sequences as set forth in SEQ ID NO: 12, SEQ ID NO: 13, and SEQ ID NO: 14, respectively) are preferably maintained.

Preferably, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 38 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 39. Thereby, the CDR sequences as defined above (heavy chain CDR1, CDR2, and CDR3 sequences as set forth in SEQ ID NO: 9, SEQ ID NO: 10, and SEQ ID NO: 11, respectively; and light chain CDR1, CDR2, and CDR3 sequences as set forth in SEQ ID NO: 12, SEQ ID NO: 13, and SEQ ID NO: 14, respectively) are preferably maintained.

In some embodiments, the antibody, or an antigen-binding fragment thereof, comprises a heavy chain variable region comprising or consisting of an amino acid sequence as set forth in SEQ ID NO: 15 and a light chain variable region comprising or consisting of an amino acid sequence as set forth in SEQ ID NO: 16.

In some embodiments, the antibody, or an antigen-binding fragment thereof, comprises a heavy chain variable region comprising or consisting of an amino acid sequence as set forth in SEQ ID NO: 15 and a light chain variable region comprising or consisting of an amino acid sequence as set forth in SEQ ID NO: 39.

In some embodiments, the antibody, or an antigen-binding fragment thereof, comprises a heavy chain variable region comprising or consisting of an amino acid sequence as set forth in SEQ ID NO: 15 and a light chain variable region comprising or consisting of an amino acid sequence as set forth in SEQ ID NO: 40.

In some embodiments, the antibody, or an antigen-binding fragment thereof, comprises a heavy chain variable region comprising or consisting of an amino acid sequence as set forth in SEQ ID NO: 37 and a light chain variable region comprising or consisting of an amino acid sequence as set forth in SEQ ID NO: 16.

In some embodiments, the antibody, or an antigen-binding fragment thereof, comprises a heavy chain variable region comprising or consisting of an amino acid sequence as set forth in SEQ ID NO: 37 and a light chain variable region comprising or consisting of an amino acid sequence as set forth in SEQ ID NO: 39.

In some embodiments, the antibody, or an antigen-binding fragment thereof, comprises a heavy chain variable region comprising or consisting of an amino acid sequence as set forth in SEQ ID NO: 37 and a light chain variable region comprising or consisting of an amino acid sequence as set forth in SEQ ID NO: 40.

In some embodiments, the antibody, or an antigen-binding fragment thereof, comprises a heavy chain variable region comprising or consisting of an amino acid sequence as set forth in SEQ ID NO: 38 and a light chain variable region comprising or consisting of an amino acid sequence as set forth in SEQ ID NO: 16.

In some embodiments, the antibody, or an antigen-binding fragment thereof, comprises a heavy chain variable region comprising or consisting of an amino acid sequence as set forth in SEQ ID NO: 38 and a light chain variable region comprising or consisting of an amino acid sequence as set forth in SEQ ID NO: 40.

Preferably, the antibody, or an antigen-binding fragment thereof, comprises a heavy chain variable region comprising or consisting of an amino acid sequence as set forth in SEQ ID NO: 38 and a light chain variable region comprising or consisting of an amino acid sequence as set forth in SEQ ID NO: 39.

In some embodiments, the antibody, or the antigen-binding fragment thereof, comprises a CDRH1 having at least 70% identity to SEQ ID NO: 17, a CDRH2 having at least 70% identity to SEQ ID NO: 18, a CDRH3 having at least 70% identity to SEQ ID NO: 51, a CDRL1 having at least 70% identity to SEQ ID NO: 20, a CDRL2 having at least 70% identity to SEQ ID NO: 21, and a CDRL3 having at least 70% identity to SEQ ID NO: 22. Preferably, the antibody or the antigen-binding fragment thereof comprises:
a heavy chain CDR1 sequence according to SEQ ID NO: 17;
a heavy chain CDR2 sequence according to SEQ ID NO: 18;
a heavy chain CDR3 sequence according to SEQ ID NO: 51;
a light chain CDR1 sequence according to SEQ ID NO: 20;
a light chain CDR2 sequence according to SEQ ID NO: 21; and
a light chain CDR3 sequence according to SEQ ID NO: 22.

As shown in the appended examples, such an antibody (e.g., 2F8 CDRH3 variant) binds specifically to Ara h 2 and Ara h 6. Moreover, as compared to wild-type 2F8, the variant 2F8 containing an engineered CDRH3 yields a more homogenous product as shown in the appended examples, thereby increasing producibility of the antibody.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 53 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 24. Thereby, the CDR sequences as defined above (heavy chain CDR1, CDR2, and CDR3 sequences as set forth in SEQ ID NO: 17, SEQ ID NO: 18, and SEQ ID NO: 51, respectively; and light chain CDR1, CDR2, and CDR3 sequences as set forth in SEQ ID NO: 20, SEQ ID NO: 21, and SEQ ID NO: 22, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 53 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 42. Thereby, the CDR sequences as defined above (heavy chain CDR1, CDR2, and CDR3 sequences as set forth in SEQ ID NO: 17, SEQ ID NO: 18, and SEQ ID NO: 51, respectively; and light chain CDR1, CDR2, and CDR3 sequences as set forth in SEQ ID NO: 20, SEQ ID NO: 21, and SEQ ID NO: 22, respectively) are preferably maintained.

Preferably, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 53 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 43. Thereby, the CDR sequences as defined above (heavy chain CDR1, CDR2, and CDR3 sequences as set forth in SEQ ID NO: 17, SEQ ID NO: 18, and SEQ ID NO: 51, respectively; and light chain CDR1, CDR2, and CDR3 sequences as set forth in SEQ ID NO: 20, SEQ ID NO: 21, and SEQ ID NO: 22, respectively) are preferably maintained.

In some embodiments, the antibody, or an antigen-binding fragment thereof, comprises a heavy chain variable region comprising or consisting of an amino acid sequence as set forth in SEQ ID NO: 53 and a light chain variable region comprising or consisting of an amino acid sequence as set forth in SEQ ID NO: 24.

In some embodiments, the antibody, or an antigen-binding fragment thereof, comprises a heavy chain variable region comprising or consisting of an amino acid sequence as set forth in SEQ ID NO: 53 and a light chain variable region comprising or consisting of an amino acid sequence as set forth in SEQ ID NO: 42.

Preferably, the antibody, or an antigen-binding fragment thereof, comprises a heavy chain variable region comprising or consisting of an amino acid sequence as set forth in SEQ ID NO: 53 and a light chain variable region comprising or consisting of an amino acid sequence as set forth in SEQ ID NO: 43.

The present invention also provides an antibody, or an antigen-binding fragment thereof, which specifically binds to Ara h 2 and/or Ara h 6, which comprises a VH having at least 70% identity to SEQ ID NO: 23; and a VL having at least 70% identity to SEQ ID NO: 42 or 43. Thereby, the heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21, and SEQ ID NO: 22, respectively, are preferably maintained. In some embodiments, such an antibody, or an antigen-binding fragment thereof, comprises a VH according to SEQ ID NO: 23; and a VL according to SEQ ID NO: 42 or 43.

The present invention also provides an antibody, or an antigen-binding fragment thereof, which specifically binds to Ara h 2 and/or Ara h 6, which comprises a VH having at least 70% identity to SEQ ID NO: 41 or 50; and a VL having at least 70% identity to SEQ ID NO: 24. Thereby, the heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21, and SEQ ID NO: 22, respectively, are preferably maintained. In some embodiments, such an antibody, or an antigen-binding fragment thereof, comprises a VH according to SEQ ID NO: 41 or 50; and a VL according to SEQ ID NO: 24.

The present invention also provides an antibody, or an antigen-binding fragment thereof, which specifically binds to Ara h 2 and/or Ara h 6, which comprises a VH having at least 70% identity to SEQ ID NO: 41 or 50; and a VL having at least 70% identity to SEQ ID NO: 42 or 43. Thereby, the heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21, and SEQ ID NO: 22, respectively, are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 41 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 42. Thereby, the CDR sequences as defined above (heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21, and SEQ ID NO: 22, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 41 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 43. Thereby, the CDR sequences as defined above (heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21, and SEQ ID NO: 22, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 50 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 42. Thereby, the CDR sequences as defined above (heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21, and SEQ ID NO: 22, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 50 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 43. Thereby, the CDR sequences as defined above (heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21, and SEQ ID NO: 22, respectively) are preferably maintained.

In some embodiments, the antibody, or an antigen-binding fragment thereof, comprises a heavy chain variable region comprising or consisting of an amino acid sequence as set forth in SEQ ID NO: 41 and a light chain variable region comprising or consisting of an amino acid sequence as set forth in SEQ ID NO: 42.

In some embodiments, the antibody, or an antigen-binding fragment thereof, comprises a heavy chain variable region comprising or consisting of an amino acid sequence as set forth in SEQ ID NO: 41 and a light chain variable region comprising or consisting of an amino acid sequence as set forth in SEQ ID NO: 43.

In some embodiments, the antibody, or an antigen-binding fragment thereof, comprises a heavy chain variable region comprising or consisting of an amino acid sequence as set forth in SEQ ID NO: 50 and a light chain variable region comprising or consisting of an amino acid sequence as set forth in SEQ ID NO: 42.

In some embodiments, the antibody, or an antigen-binding fragment thereof, comprises a heavy chain variable region comprising or consisting of an amino acid sequence as set forth in SEQ ID NO: 50 and a light chain variable region comprising or consisting of an amino acid sequence as set forth in SEQ ID NO: 43.

In some embodiments, the antibody, or the antigen-binding fragment thereof, comprises a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 52, and a CDRL3 having at least 70% identity to SEQ ID NO: 30. Preferably, the antibody or the antigen-binding fragment thereof comprises:
a heavy chain CDR1 sequence according to SEQ ID NO: 25;
a heavy chain CDR2 sequence according to SEQ ID NO: 26;
a heavy chain CDR3 sequence according to SEQ ID NO: 27;
a light chain CDR1 sequence according to SEQ ID NO: 28;
a light chain CDR2 sequence according to SEQ ID NO: 52; and
a light chain CDR3 sequence according to SEQ ID NO: 30.

As shown in the appended examples, such an antibody (e.g., 7G6 CDRL2 variant) binds specifically to Ara h 2 and Ara h 6. Moreover, as compared to wild-type 7G6, the variant 7G6 containing an engineered CDRL2 exhibits less unspecific binding as shown in the appended examples, thereby increasing producibility of the antibody.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 31 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 54. Thereby, the CDR sequences as defined above (heavy chain CDR1, CDR2, and CDR3 sequences as set forth in SEQ ID NO: 25, SEQ ID NO: 26, and SEQ ID NO: 27, respectively; and light chain CDR1, CDR2, and CDR3 sequences as set forth in SEQ ID NO: 28, SEQ ID NO: 29, and SEQ ID NO: 30, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 33 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 54. Thereby, the CDR sequences as defined above (heavy chain CDR1, CDR2, and CDR3 sequences as set forth in SEQ ID NO: 25, SEQ ID NO: 26, and SEQ ID NO: 27, respectively; and light chain CDR1, CDR2, and CDR3 sequences as set forth in SEQ ID NO: 28, SEQ ID NO: 29, and SEQ ID NO: 30, respectively) are preferably maintained.

Preferably, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 34 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 54. Thereby, the CDR sequences as defined above (heavy chain CDR1, CDR2, and CDR3 sequences as set forth in SEQ ID NO: 25, SEQ ID NO: 26, and SEQ ID NO: 27, respectively; and light chain CDR1, CDR2, and CDR3 sequences as set forth in SEQ ID NO: 28, SEQ ID NO: 29, and SEQ ID NO: 30, respectively) are preferably maintained.

In some embodiments, the antibody, or an antigen-binding fragment thereof, comprises a heavy chain variable region comprising or consisting of an amino acid sequence as set forth in SEQ ID NO: 31 and a light chain variable region comprising or consisting of an amino acid sequence as set forth in SEQ ID NO: 54.

In some embodiments, the antibody, or an antigen-binding fragment thereof, comprises a heavy chain variable region comprising or consisting of an amino acid sequence as set forth in SEQ ID NO: 33 and a light chain variable region comprising or consisting of an amino acid sequence as set forth in SEQ ID NO: 54.

Preferably, the antibody, or an antigen-binding fragment thereof, comprises a heavy chain variable region comprising or consisting of an amino acid sequence as set forth in SEQ ID NO: 34 and a light chain variable region comprising or consisting of an amino acid sequence as set forth in SEQ ID NO: 54.

The present invention also provides an antibody, or an antigen-binding fragment thereof, which specifically binds to Ara h 2 and/or Ara h 6, which comprises a VH having at least 70% identity to SEQ ID NO: 31; and a VL having at least 70% identity to SEQ ID NO: 35 or 36. Thereby, the heavy chain CDR1, CDR2, and CDR3 sequences as set forth in SEQ ID NO: 25, SEQ ID NO: 26, and SEQ ID NO: 27, respectively; and light chain CDR1, CDR2, and CDR3 sequences as set forth in SEQ ID NO: 28, SEQ ID NO: 29, and SEQ ID NO: 30, respectively, are preferably maintained. In some embodiments, such an antibody, or an antigen-binding fragment thereof, comprises a VH according to SEQ ID NO: 31; and a VL according to SEQ ID NO: 35 or 36.

The present invention also provides an antibody, or an antigen-binding fragment thereof, which specifically binds to Ara h 2 and/or Ara h 6, which comprises a VH having at least 70% identity to SEQ ID NO: 33 or 34; and a VL having at least 70% identity to SEQ ID NO: 32. Thereby, the heavy chain CDR1, CDR2, and CDR3 sequences as set forth in SEQ ID NO: 25, SEQ ID NO: 26, and SEQ ID NO: 27, respectively; and light chain CDR1, CDR2, and CDR3 sequences as set forth in SEQ ID NO: 28, SEQ ID NO: 29, and SEQ ID NO: 30, respectively, are preferably maintained. In some embodiments, such an antibody, or an antigen-binding fragment thereof, comprises a VH according to SEQ ID NO: 33 or 34; and a VL according to SEQ ID NO: 32.

The present invention also provides an antibody, or an antigen-binding fragment thereof, which specifically binds to Ara h 2 and/or Ara h 6, which comprises a VH having at least 70% identity to SEQ ID NO: 33 or 34; and a VL having at least 70% identity to SEQ ID NO: 35 or 36. Thereby, the heavy chain CDR1, CDR2, and CDR3 sequences as set forth in SEQ ID NO: 25, SEQ ID NO: 26, and SEQ ID NO: 27, respectively; and light chain CDR1, CDR2, and CDR3 sequences as set forth in SEQ ID NO: 28, SEQ ID NO: 29, and SEQ ID NO: 30, respectively, are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 33 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 35. Thereby, the CDR sequences as defined above (heavy chain CDR1, CDR2, and CDR3 sequences as set forth in SEQ ID NO: 25, SEQ ID NO: 26, and SEQ ID NO: 27, respectively; and light chain CDR1, CDR2, and CDR3 sequences as set forth in SEQ ID NO: 28, SEQ ID NO: 29, and SEQ ID NO: 30, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 33 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 36. Thereby, the CDR sequences as defined above (heavy chain CDR1, CDR2, and CDR3 sequences as set forth in SEQ ID NO: 25, SEQ ID NO: 26, and SEQ ID NO: 27, respectively; and light chain CDR1, CDR2, and CDR3 sequences as set forth in SEQ ID NO: 28, SEQ ID NO: 29, and SEQ ID NO: 30, respectively) are preferably maintained.

In some embodiments, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 34 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 36. Thereby, the CDR sequences as defined above (heavy chain CDR1, CDR2, and CDR3 sequences as set forth in SEQ ID NO: 25, SEQ ID NO: 26, and SEQ ID NO: 27, respectively; and light chain CDR1, CDR2, and CDR3 sequences as set forth in SEQ ID NO: 28, SEQ ID NO: 29, and SEQ ID NO: 30, respectively) are preferably maintained.

Preferably, such an antibody of the invention, or the antigen-binding fragment thereof, may comprise (i) a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 34 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 35. Thereby, the CDR sequences as defined above (heavy chain CDR1, CDR2, and CDR3 sequences as set forth in SEQ ID NO: 25, SEQ ID NO: 26, and SEQ ID NO: 27, respectively; and light chain CDR1, CDR2, and CDR3 sequences as set forth in SEQ ID NO: 28, SEQ ID NO: 29, and SEQ ID NO: 30, respectively) are preferably maintained.

In some embodiments, the antibody, or an antigen-binding fragment thereof, comprises a heavy chain variable region comprising or consisting of an amino acid sequence as set forth in SEQ ID NO: 33 and a light chain variable region comprising or consisting of an amino acid sequence as set forth in SEQ ID NO: 35.

In some embodiments, the antibody, or an antigen-binding fragment thereof, comprises a heavy chain variable region comprising or consisting of an amino acid sequence as set forth in SEQ ID NO: 33 and a light chain variable region comprising or consisting of an amino acid sequence as set forth in SEQ ID NO: 36.

In some embodiments, the antibody, or an antigen-binding fragment thereof, comprises a heavy chain variable region comprising or consisting of an amino acid sequence as set forth in SEQ ID NO: 34 and a light chain variable region comprising or consisting of an amino acid sequence as set forth in SEQ ID NO: 36.

Preferably, the antibody, or an antigen-binding fragment thereof, comprises a heavy chain variable region comprising or consisting of an amino acid sequence as set forth in SEQ ID NO: 34 and a light chain variable region comprising or consisting of an amino acid sequence as set forth in SEQ ID NO: 35.

In general, the antibody, or the antigen-binding fragment thereof, of the invention may be capable of reducing, inhibiting or neutralizing allergen-mediated biological activity. In particular, the antibodies may be capable of reducing or inhibiting the binding of an IgE antibody to a peanut allergen, in particular Ara h 2, Ara h 3 and/or Ara h 6 as described herein. Thus, the antibodies or binding fragments thereof according to the invention may decrease or inhibit the activation of the mast cells or basophils and therefore decrease or prevent the release of mediators (e.g. histamine, lipid mediators, leukotriene). Thereby, the antibodies described herein may inhibit allergy symptoms that would usually occur in the patient after contact with the allergen (e.g. contact with the eyes, nose or mouth or food uptake). Accordingly, the antibodies described herein may be capable of reducing, inhibiting or neutralizing allergen-mediated biological activity. In particular, the antibodies may be capable of reducing or inhibiting the binding of an IgE antibody to a peanut allergen, in particular Ara h 2, Ara h 3 and/or Ara h 6.

In some embodiments, the CDRs or the variable regions of the antibody, or the antigen-binding fragment thereof, are human or are derived from human CDR or variable region sequences. The exemplary antibodies 2F8, 7G6, 17H9 and 15E3 (wild-type) are human antibodies, isolated from human patients. A “human-derived” CDR or VH/VL sequence includes engineered human antibody sequences, wherein mutations were introduced in the originally human CDR or VH/VL sequences. For example, a human-derived CDR may differ from the fully human (wild-type) CDR sequence in that it contains up to 5, i.e. 1, 2, 3, 4 or 5 mutations, preferably up to 4 mutations, more preferably up to 3 mutations. For example, a human-derived VH or VL sequence may differ from the fully human (wild-type) VH or VL sequence in that it contains up to 10, i.e. 1, 2, 3, 4, 5, 6, 7, 8, 9 or 10 mutations, preferably up to 7 mutations, more preferably up to 5 mutations (e.g., in the framework regions).

In some embodiments, the antibody, or the antigen-binding fragment thereof, is a human antibody. In some embodiments, the antibody, or the antigen-binding fragment thereof, is a monoclonal antibody. For example, the antibody, or the antigen-binding fragment thereof, may be a human monoclonal antibody.

Human antibodies are advantageous as compared to antibodies of non-human origin, because non-human antibodies, including chimeric and humanized antibodies, can trigger an adverse immune response, which can lead to nausea, diarrhea and flu-like symptoms. In more severe cases, these side-effects can even be lethal. Non-human antibody segments often trigger immune responses in humans (anti-drug antibodies (ADA)), thereby not only eliciting undesired side effects, but also reducing the efficacy of the non-human antibody in humans. In contrast thereto, antibodies retrieved from humans have a higher safety profile, as the antibodies have proven tolerability in the human body, which is combined with the outstanding affinity maturation typical of the human immune system. As used herein, the term “human antibodies” not only includes antibodies originally found in humans, but also sequence variants thereof, wherein specific amino acid residues (but not entire antibody segments) are mutated. In contrast to non-human and humanized antibodies, which usually contain entire antibody segments (such as entire sets of CDR sequences) of non-human origin, sequence variants of human antibodies typically contain only selective/specific mutations within select antibody segments (e.g., within a CDR or framework region and/or within a constant region; e.g. to modify the antibodies' affinity, functionality, half-life, etc.).

For example, a human antibody according to the present invention may comprise only a limited number of mutations per CDR (e.g. no more than 6, preferably no more than 5, more preferably no more than 4, even more preferably no more than 3, still more preferably no more than 2 and particularly preferably only a single mutation per CDR), as compared to the sequences shown in Table 1. In case of more than two mutations they may not occur in a consecutive manner (to avoid creating a non-human sequence segment). The same applies to the framework regions (or the entire VH/VL sequences) as well to the constant regions. The latter may carry, for example, specific modifications known in the art to modify the antibody's (Fc-related) functionality, as described herein below.

Preferably, the antibody is an IgG or IgA antibody. IgG and IgA usually compete with IgE for binding sites on the allergen and thereby prevent recognition of allergens by IgE bound to Fes receptors on the surface of mast cells and basophils. This may include direct competition by binding to the same epitope or competition through steric hindrance. Furthermore, IgG antibodies bound to the allergen can lead to cross-linking of Fcs and inhibitory FcγRIIB receptors, resulting in the decrease of effector cell activity. Thereby the IgG and IgA antibodies or binding fragments thereof according to the invention can be used for the effective prevention or treatment of allergies. In some embodiments, the variable regions or the CDRs of the antibody as defined herein are derived from a (human) IgE antibody and grafted in a scaffold of an IgG or IgA antibody. Preferably, the scaffold is of a human IgG or IgA. Accordingly, the variable regions, portions thereof or the CDRs may be human and grafted in an antibody framework, which is preferably of human origin, but a distinct antibody type, such as IgG or IgA instead of IgE. Typically, the human-derived portions of the variable regions that are grafted into the antibody framework comprise the CDRs. Among IgG, IgG1 and IgG4 are preferred.

Accordingly, the antibody according to the present invention, or an antigen binding fragment thereof, may comprise an Fc moiety. The Fc moiety may be derived from human origin, e.g. from human IgA or IgG, such as IgG1, IgG2, IgG3, and/or IgG4, e.g. human IgG1.

As used herein, the term “Fc moiety” refers to a sequence derived from the portion of an immunoglobulin heavy chain beginning in the hinge region just upstream of the papain cleavage site (e.g., residue 216 in native IgG, taking the first residue of heavy chain constant region to be 114) and ending at the C-terminus of the immunoglobulin heavy chain. Accordingly, an Fc moiety may be a complete Fc moiety or a portion (e.g., a domain) thereof. A complete Fc moiety comprises at least a hinge domain, a CH2 domain, and a CH3 domain (e.g., EU amino acid positions 216-446). An additional lysine residue (K) is sometimes present at the extreme C-terminus of the Fc moiety, but is often cleaved from a mature antibody.

In some embodiments, in the context of the present invention an Fc moiety comprises at least one of: a hinge (e.g., upper, middle, and/or lower hinge region) domain, a CH2 domain, a CH3 domain, or a variant, portion, or fragment thereof. An Fc moiety may comprise at least a hinge domain, a CH2 domain or a CH3 domain. The Fc moiety may be a complete Fc moiety. The Fc moiety may also comprises one or more amino acid insertions, deletions, or substitutions relative to a naturally-occurring Fc moiety. For example, at least one of a hinge domain, CH2 domain or CH3 domain (or portion thereof) may be deleted.

It will be understood by one of ordinary skill in the art that the Fc moiety may be modified such that it varies in amino acid sequence from the complete Fc moiety of a naturally occurring immunoglobulin molecule, while retaining at least one desirable function conferred by the naturally-occurring Fc moiety. Such functions include Fc receptor (FcR) binding, antibody half-life modulation, ADCC function, protein A binding, protein G binding, and complement binding. The portions of naturally occurring Fc moieties, which are responsible and/or essential for such functions are well known by those skilled in the art. In some embodiments, the antibody according to the present invention comprises a (complete) Fc moiety/Fc region, wherein the interaction/binding with the Fc receptor is not compromised.

In general, binding of the antibody to an Fc receptor may be assessed by various methods known to the skilled person, such as ELISA (Hessell A J, Hangartner L, Hunter M, Havenith C E G, Beurskens F J, Bakker J M, Lanigan C M S, Landucci G, Forthal D N, Parren P W H I, et al.: Fc receptor but not complement binding is important in antibody protection against HIV. Nature 2007, 449:101-104; Grevys A, Bern M, Foss S, Bratlie D B, Moen A, Gunnarsen K S, Aase A, Michaelsen T E, Sandlie I, Andersen J T: Fc Engineering of Human IgG1 for Altered Binding to the Neonatal Fc Receptor Affects Fc Effector Functions. 2015, 194:5497-5508) or flow-cytometry (Perez L G, Costa M R, Todd C A, Haynes B F, Montefiori D C: Utilization of immunoglobulin G Fc receptors by human immunodeficiency virus type 1: a specific role for antibodies against the membrane-proximal external region of gp41. J Virol 2009, 83:7397-7410; Piccoli L, Campo I, Fregni C S, Rodriguez B M F, Minola A, Sallusto F, Luisetti M, Corti D, Lanzavecchia A: Neutralization and clearance of GM-CSF by autoantibodies in pulmonary alveolar proteinosis. Nat Commun 2015, 6:1-9).

In some embodiments, the antibody, or antigen binding fragment thereof, according to the present invention comprises an Fc region. As used herein, the term “Fc region” refers to the portion of an immunoglobulin formed by two or more Fc moieties of antibody heavy chains. For example, the Fc region may be monomeric or “single-chain” Fc region (i.e., a scFc region). Single chain Fc regions are comprised of Fc moieties linked within a single polypeptide chain (e.g., encoded in a single contiguous nucleic acid sequence). Exemplary scFc regions are disclosed in WO 2008/143954 A2. The Fc region may be dimeric. A “dimeric Fc region” or “dcFc” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties.

In some embodiments, the Fc moiety, or the Fc region, comprises or consists of an amino acid sequence derived from a human immunoglobulin sequence (e.g., from an Fc region or Fc moiety from a human IgG molecule). However, the Fc moiety, or the Fc region, may comprise one or more amino acids from another mammalian species. For example, a primate Fc moiety or a primate binding site may be included in the antibody, or antigen-binding fragment. Alternatively, one or more murine amino acids may be present in the Fc moiety or in the Fc region.

The Fc moieties of the Fc region may be of the same or different class and/or subclass. For example, the Fc moieties may be derived from an immunoglobulin (e.g., a human immunoglobulin) of an IgG1, IgG2, IgG3 or IgG4 subclass.

Accordingly, antibodies of the invention can be of any isotype (e.g., IgA, IgG, IgM i.e. an a, Y or u heavy chain). Preferably, the antibody may be of the IgA or IgG type. Within the IgG isotype, antibodies may be IgG1, IgG2, IgG3 or IgG4 subclass, preferably IgG1 or IgG4. Exemplified sequences for IgG1 and IgG4 constant regions, which may be useful in the antibody as described herein, are provided in SEQ ID NO: 59 (IgG1) and SEQ ID NO: 60 (IgG4). Accordingly, the antibody of the invention may comprise an amino acid sequence according to SEQ ID NO: 59 or 60, or a sequence variant thereof as described herein. The human IgG4 constant region sequence of SEQ ID NO: 60 comprises the stable hinge mutation S228P (S. Angal, D. J. King, M. W. Bodmer, A. Turner, A. D. G. Lawson, G. Roberts, B. Pedley, J. R. Adair, A single amino acid substitution abolishes the heterogeneity of chimeric mouse/human (IgG4) antibody, Molecular Immunology, Volume 30, Issue 1, 1993, Pages 105-108, ISSN 0161-5890, https://doi.org/10.1016/0161-5890 (93) 90432-B). Antibodies of the invention may have a κ or a λ light chain. Exemplified sequences for x and λ light chain constant regions, which may be useful in the antibody as described herein, are provided in SEQ ID NO: 61 (Ckappa) and SEQ ID NO: 62 (Clambda). Accordingly, the antibody of the invention may comprise an amino acid sequence according to SEQ ID NO: 61 or 62, or a sequence variant thereof as described herein.

As outlined above, the present invention encompasses antigen-binding fragments. An antigen-binding fragment may or may not comprise an Fc moiety, in particular a portion of a complete Fc region. In some embodiments, the antibody, or antigen-binding fragment thereof, is selected from Fab, Fab′, F(ab′)2, Fv or scFv. For example, F(ab′)2 (which may be obtained by pepsin cleavage or recombinant expression) as well as Fab′ (which can be obtained from F(ab′)2 or by recombinant expression) usually includes the hinge region. An exemplified CH1 sequence, which may be used, e.g., as constant region in a Fab, is provided in SEQ ID NO: 63. Accordingly, the antibody of the invention, or the antigen-binding fragment thereof, may comprise an amino acid sequence according to SEQ ID NO: 63, or a sequence variant thereof as described herein.

In some embodiments, the antibody, or antigen-binding fragment, may be a single-chain antibody (or fragment). The single-chain antibody (or fragment) may encode the complete set of six CDRs, i.e. include the three heavy chain CDRs as well as the three light chain CDRs. More specifically, the single-chain antibody (or fragment) may include a heavy chain variable region (VH) as well as a light chain variable region (VL), for example including the VH and VL sequences as described above.

Variant antibodies are also included within the scope of the invention. Thus, variants of the sequences recited in the application are also included within the scope of the invention. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Antibodies of the invention, or antigen-binding fragments thereof, may be provided in purified form. Typically, the antibody, or antigen-binding fragment, will be present in a composition that is substantially free of other polypeptides e.g., where less than 90% (by weight), usually less than 60% and more usually less than 50% of the composition is made up of other polypeptides.

Antibodies of the invention may be immunogenic in non-human (or heterologous) hosts e.g., in mice. In particular, the antibodies may have an idiotope that is immunogenic in non-human hosts, but not in a human host. In particular, antibodies of the invention for human use include those that cannot be easily isolated from hosts such as mice, goats, rabbits, rats, non-primate mammals, etc. and cannot generally be obtained by humanization or from xeno-mice.

Antibodies of the invention also include hybrid antibody molecules that comprise the six CDRs from an antibody of the invention as defined above and one or more CDRs from another antibody to an antigen. For example, the antibody may be multispecific. In other embodiments, the antibody, or the antigen-binding fragment thereof, may be monospecific.

### Multispecific Antibodies or Antigen-Binding Fragments

Accordingly, the antibody as described herein, or the antigen-binding fragment thereof, may be a multispecific antibody or a multispecific antigen-binding fragment.

As used herein, the term “multispecific” refers to the ability to bind to at least two different epitopes, e.g. on different antigens or on the same antigen. While a conventional monospecific IgG-type antibodies usually have two identical epitope binding sites (paratopes) and can, thus, only bind to identical epitopes (but not to different epitopes). A multispecific antibody, in contrast, has at least two different types of paratopes (antigen-binding sites) and can, thus, bind to at least two different epitopes. As used herein, “paratope” refers to an antigen-binding site (or epitope-binding site) of the antibody. Moreover, a single “specificity” may refer to one, two, three or more identical paratopes in a single antibody (the actual number of paratopes in one single antibody molecule is referred to as “valency”). For example, a single native IgG antibody is monospecific and bivalent, since it has two identical paratopes. Accordingly, a multispecific antibody comprises at least two (different) paratopes. Thus, the term “multispecific antibodies” refers to antibodies having more than one paratope and the ability to bind to two or more different epitopes. The term “multispecific antibodies” comprises in particular bispecific antibodies, but typically also protein, e.g. antibody scaffolds, which bind in particular to three or more different epitopes, i.e. antibodies with three or more different paratopes.

In particular, the multispecific antibody, or the multispecific antigen binding fragment, of the invention may comprise two or more paratopes, wherein one or more paratopes may be identical so that all paratopes of the antibody belong to at least two different types of paratopes and, hence, the antibody has at least two specificities. For example, the multispecific antibody or antigen binding fragment thereof according to the present invention may comprise four paratopes, wherein each two paratopes are identical (i.e. have the same specificity) and, thus, the antibody or fragment thereof is bispecific and tetravalent (two identical paratopes for each of the two specificities). Thus, “one specificity” refers in particular to one or more paratopes exhibiting the same specificity (which typically means that such one or more paratopes are identical) and, thus, “two specificities” may be realized by two, three, four five, six or more paratopes as long as they refer to only two specificities. In some embodiments, the multispecific antibody comprises one single paratope for each (of the at least two) specificity, i.e. the multispecific antibody comprises in total at least two paratopes. For example, a bispecific antibody may comprise one single paratope for each of the two specificities, i.e. the antibody comprises in total two paratopes. In other embodiments, the antibody comprises two (identical) paratopes for one or more of the specificities.

Preferably, the multispecific antibody or the multispecific antigen-binding fragment is bispecific, trispecific or tetraspecific. As used herein, terms like “bispecific”, trispecific”, “tetraspecific” etc. refer to the number of different epitopes to which the antibody can bind to. For example, a “bispecific” antibody has exactly two different specificities (two different antigen-binding sites, wherein each of the two different antigen-binding sites may independently occur once or more than once, e.g. twice). For example, a “trispecific” antibody has exactly three different specificities (three different antigen-binding sites, wherein each of the three different antigen-binding sites may independently occur once or more than once, e.g. twice). For example, a “tetraspecific” antibody has exactly four different specificities (four different antigen-binding sites, wherein each of the four different antigen-binding sites may independently occur once or more than once, e.g. twice).

Various such multispecific antibody formats and methods for obtaining multispecific antibodies are known in the art. For example, building blocks for bispecific and trispecific antibodies are described in Xiufeng Wu, Stephen J. Demarest, Building blocks for bispecific and trispecific antibodies, Methods, Volume 154, 2019, Pages 3-9, ISSN 1046-2023, https://doi.org/10.1016/j.ymeth.2018.08.010, which is incorporated herein by reference. Methods for obtaining multispecific antibodies and further multispecific antibody formats are described in Amaral M, Hölper S, Lange C, Jung J, Sjuts H, Weil S, Fischer M, Radoevic K, Rao E. Engineered Technologies and Bioanalysis of multispecific antibody formats. J Appl Bioanal 6 (1), 26-51 (2020), which is incorporated herein by reference.

In some embodiments, the multispecific antibody, or the multispecific antigen binding fragment, according to the present invention is a bispecific antibody or a bispecific antigen binding fragment. Bispecific antibodies comprise (exactly) two specificities. A bispecific antibody in the context of the present invention may be of any bispecifc antibody format known in the art, e.g., as described in Spiess C., Zhai Q. and Carter P. J. (2015) Molecular Immunology 67:95-106. For example, bispecific antibodies may be whole antibodies, such as whole IgG-like molecules, or fragments thereof which are not whole antibodies but retain antibody properties. These may be small recombinant formats, e.g. as tandem single chain variable fragment molecules (taFvs), diabodies (Dbs), single chain diabodies (scDbs), and various other derivatives of these (e.g., as described in Byrne H. et al. (2013) Trends Biotech, 31 (11): 621-632 with FIG. 2 showing various bispecific antibody formats). In some embodiments, the bispecific antibody may be an IgG (H)-scFv fusion as described in Coloma, M., Morrison, S. Design and production of novel tetravalent bispecific antibodies. Nat Biotechnol 15, 159-163 (1997). https://doi.org/10.1038/nbt0297-159. Preferred bispecific antibody formats are those as described in Example 6.

In some embodiments, the multispecific antibody, or the multispecific antigen binding fragment, according to the present invention is a trispecific antibody or a trispecific antigen binding fragment. Trispecific antibodies comprise (exactly) three specificities. A trispecific antibody in the context of the present invention may be of any trispecifc antibody format known in the art. In some embodiments, the trispecific antibody may be a heterodimer of IgG (H)-scFvs with same Fab domains using knob-into-hole CH3 as described in Ridgway J B, Presta L G, Carter P. ‘Knobs-into-holes’ engineering of antibody CH3 domains for heavy chain heterodimerization. Protein Eng. 1996 July; 9 (7): 617-21. doi: 10.1093/protein/9.7.617. A preferred trispecific antibody format is the trispecific format as described in Example 6.

In some embodiments, the multispecific antibody, or the multispecific antigen binding fragment, according to the present invention is a tetraspecific antibody or a tetraspecific antigen binding fragment. Tetraspecific antibodies comprise (exactly) four specificities. A tetraspecific antibody in the context of the present invention may be of any tetraspecifc antibody format known in the art. In some embodiments, the tetraspecific antibody may be a a heterodimer of IgG (H)-scFv and scFv-Fc-scFv using knob-into-hole CH3 as described in Ridgway J B, Presta L G, Carter P. ‘Knobs-into-holes’ engineering of antibody CH3 domains for heavy chain heterodimerization. Protein Eng. 1996 July; 9 (7): 617-21. doi: 10.1093/protein/9.7.617. In other embodiments, the tetraspecific antibody may be an IgG4 heavy chain heterodimer. Preferred tetraspecific antibody formats are those as described in Example 6.

Usually, the multispecific antibody, or the multispecific antigen binding fragment, is at least bivalent, i.e. it has at least two paratopes. Preferably, the multispecific antibody, or the multispecific antigen binding fragment, is bivalent, trivalent, tetravalent, or hexavalent. More preferably, the multispecific antibody, or the multispecific antigen binding fragment, is tetravalent. Even more preferably, the multispecific antibody, or the multispecific antigen binding fragment, is tetravalent and bispecific, trispecific or tetraspecific.

Preferably, the multispecific antibody, or the multispecific antigen binding fragment, binds (specifically) to distinct, non-overlapping epitopes of Ara h 2. Accordingly, the antigen-binding sites (paratopes) of the multispecific antibody, or the multispecific antigen binding fragment, preferably target different, non-overlapping epitopes on the same antigen, namely Ara h 2. To this end, the antigen-binding sites (paratopes) of the multispecific antibody, or the multispecific antigen binding fragment, may be selected from the four different antibodies 17H9, 15E3, 2F8 and 7G6, or the variants thereof, as described above. As shown in the appended examples, 17H9, 15E3, 2F8 and 7G6 bind to distinct, non-overlapping epitopes of Ara h 2. The skilled artisan is well aware of methods for obtaining a multispecific antibody using the binding sites of monospecific antibodies, e.g. as described above.

Accordingly, the multispecific antibody, or the multispecific antigen binding fragment, may comprise at least two of the following:
(i) an antigen-binding site comprising (a) the CDRH1, CDRH2, CDRH3, CDRL1, CDRL2 and CDRL3 sequences and/or (b) the VH and VL sequences of 17H9, or sequence variants thereof, as described above;
(ii) an antigen-binding site comprising (a) the CDRH1, CDRH2, CDRH3, CDRL1, CDRL2 and CDRL3 sequences and/or (b) the VH and VL sequences of 15E3, or sequence variants thereof, as described above;
(iii) an antigen-binding site comprising (a) the CDRH1, CDRH2, CDRH3, CDRL1, CDRL2 and CDRL3 sequences and/or (b) the VH and VL sequences of 2F8, or sequence variants thereof, as described above; and/or
(iv) an antigen-binding site comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the multispecific antibody, or the multispecific antigen binding fragment, may comprise (i) an antigen-binding site comprising (a) the CDRH1, CDRH2, CDRH3, CDRL1, CDRL2 and CDRL3 sequences and/or (b) the VH and VL sequences of 17H9, or sequence variants thereof, as described above; and (ii) an antigen-binding site comprising (a) the CDRH1, CDRH2, CDRH3, CDRL1, CDRL2 and CDRL3 sequences and/or (b) the VH and VL sequences of (wild-type) 2F8, or sequence variants thereof, as described herein, e.g. in Tables 1 and 2. Optionally, such an antibody may further comprise an antigen-binding site including the CDR and/or VH/VL sequences of 15E3 (or a sequence variant thereof), and/or an antigen-binding site including the CDR and/or VH/VL sequences of 7G6 (or a sequence variant thereof).

In some embodiments, the multispecific antibody, or the multispecific antigen binding fragment, may comprise (i) an antigen-binding site comprising (a) the CDRH1, CDRH2, CDRH3, CDRL1, CDRL2 and CDRL3 sequences and/or (b) the VH and VL sequences of 15E3, or sequence variants thereof, as described above; and (ii) an antigen-binding site comprising (a) the CDRH1, CDRH2, CDRH3, CDRL1, CDRL2 and CDRL3 sequences and/or (b) the VH and VL sequences of (wild-type) 7G6, or sequence variants thereof, as described herein, e.g. in Tables 1 and 2. Optionally, such an antibody may further comprise an antigen-binding site including the CDR and/or VH/VL sequences of 17H9 (or a sequence variant thereof), and/or an antigen-binding site including the CDR and/or VH/VL sequences of 2F8 (or a sequence variant thereof).

In some embodiments, the multispecific antibody, or the multispecific antigen binding fragment, may comprise (i) an antigen-binding site comprising (a) the CDRH1, CDRH2, CDRH3, CDRL1, CDRL2 and CDRL3 sequences and/or (b) the VH and VL sequences of 17H9, or sequence variants thereof, as described above; and (ii) an antigen-binding site comprising (a) the CDRH1, CDRH2, CDRH3, CDRL1, CDRL2 and CDRL3 sequences and/or (b) the VH and VL sequences of 15E3, or sequence variants thereof, as described herein, e.g. in Tables 1 and 2. Optionally, such an antibody may further comprise an antigen-binding site including the CDR and/or VH/VL sequences of 2F8 (or a sequence variant thereof), and/or an antigen-binding site including the CDR and/or VH/VL sequences of 7G6 (or a sequence variant thereof).

Preferably, the multispecific antibody, or the multispecific antigen binding fragment, may comprise (i) an antigen-binding site comprising (a) the CDRH1, CDRH2, CDRH3, CDRL1, CDRL2 and CDRL3 sequences and/or (b) the VH and VL sequences of 15E3, or sequence variants thereof, as described above; and (ii) an antigen-binding site comprising (a) the CDRH1, CDRH2, CDRH3, CDRL1, CDRL2 and CDRL3 sequences and/or (b) the VH and VL sequences of (wild-type) 2F8, or sequence variants thereof, as described herein, e.g. in Tables 1 and 2. Optionally, such an antibody may further comprise an antigen-binding site including the CDR and/or VH/VL sequences of 17H9 (or a sequence variant thereof), and/or an antigen-binding site including the CDR and/or VH/VL sequences of 7G6 (or a sequence variant thereof).

More preferably, the multispecific antibody, or the multispecific antigen binding fragment, may comprise (i) an antigen-binding site comprising (a) the CDRH1, CDRH2, CDRH3, CDRL1, CDRL2 and CDRL3 sequences and/or (b) the VH and VL sequences of 17H9, or sequence variants thereof, as described above; and (ii) an antigen-binding site comprising (a) the CDRH1, CDRH2, CDRH3, CDRL1, CDRL2 and CDRL3 sequences and/or (b) the VH and VL sequences of (wild-type) 7G6, or sequence variants thereof, as described herein, e.g. in Tables 1 and 2. Optionally, such an antibody may further comprise an antigen-binding site including the CDR and/or VH/VL sequences of 15E3 (or a sequence variant thereof), and/or an antigen-binding site including the CDR and/or VH/VL sequences of 2F8 (or a sequence variant thereof).

In some embodiments, the multispecific antibody, or the multispecific antigen binding fragment, may be a bispecific antibody or a bispecific antigen binding fragment comprising:
(i) an antigen-binding site comprising (a) the CDRH1, CDRH2, CDRH3, CDRL1, CDRL2 and CDRL3 sequences and/or (b) the VH and VL sequences of 17H9, or sequence variants thereof, as described above; and
(ii) an antigen-binding site comprising (a) the CDRH1, CDRH2, CDRH3, CDRL1, CDRL2 and CDRL3 sequences and/or (b) the VH and VL sequences of 15E3, or sequence variants thereof, as described above.

In some embodiments, the multispecific antibody, or the multispecific antigen binding fragment, may be a bispecific antibody or a bispecific antigen binding fragment comprising:
(i) an antigen-binding site comprising (a) the CDRH1, CDRH2, CDRH3, CDRL1, CDRL2 and CDRL3 sequences and/or (b) the VH and VL sequences of 17H9, or sequence variants thereof, as described above; and
(iii) an antigen-binding site comprising (a) the CDRH1, CDRH2, CDRH3, CDRL1, CDRL2 and CDRL3 sequences and/or (b) the VH and VL sequences of 2F8, or sequence variants thereof, as described above.

In some embodiments, the multispecific antibody, or the multispecific antigen binding fragment, may be a bispecific antibody or a bispecific antigen binding fragment comprising:
(ii) an antigen-binding site comprising (a) the CDRH1, CDRH2, CDRH3, CDRL1, CDRL2 and CDRL3 sequences and/or (b) the VH and VL sequences of 15E3, or sequence variants thereof, as described above; and
(iv) an antigen-binding site comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the multispecific antibody, or the multispecific antigen binding fragment, may be a bispecific antibody or a bispecific antigen binding fragment comprising:
(iii) an antigen-binding site comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antigen-binding site comprising (a) the CDRH1, CDRH2, CDRH3, CDRL1, CDRL2 and CDRL3 sequences and/or (b) the VH and VL sequences of 7G6, or sequence variants thereof, as described above.

Preferably, the multispecific antibody, or the multispecific antigen binding fragment, may be a bispecific antibody or a bispecific antigen binding fragment comprising:
(ii) an antigen-binding site comprising (a) the CDRH1, CDRH2, CDRH3, CDRL1, CDRL2 and CDRL3 sequences and/or (b) the VH and VL sequences of 15E3, or sequence variants thereof, as described above; and
(iii) an antigen-binding site comprising (a) the CDRH1, CDRH2, CDRH3, CDRL1, CDRL2 and CDRL3 sequences and/or (b) the VH and VL sequences of 2F8, or sequence variants thereof, as described above.

More preferably, the multispecific antibody, or the multispecific antigen binding fragment, may be a bispecific antibody or a bispecific antigen binding fragment comprising:
(i) an antigen-binding site comprising (a) the CDRH1, CDRH2, CDRH3, CDRL1, CDRL2 and CDRL3 sequences and/or (b) the VH and VL sequences of 17H9, or sequence variants thereof, as described above; and
(iv) an antigen-binding site comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the multispecific antibody, or the multispecific antigen binding fragment, may be a trispecific antibody or a trispecific antigen binding fragment comprising:
(i) an antigen-binding site comprising (a) the CDRH1, CDRH2, CDRH3, CDRL1, CDRL2 and CDRL3 sequences and/or (b) the VH and VL sequences of 17H9, or sequence variants thereof, as described above;
(ii) an antigen-binding site comprising (a) the CDRH1, CDRH2, CDRH3, CDRL1, CDRL2 and CDRL3 sequences and/or (b) the VH and VL sequences of 15E3, or sequence variants thereof, as described above; and
(iii) an antigen-binding site comprising (a) the CDRH1, CDRH2, CDRH3, CDRL1, CDRL2 and CDRL3 sequences and/or (b) the VH and VL sequences of 2F8, or sequence variants thereof, as described above.

In some embodiments, the multispecific antibody, or the multispecific antigen binding fragment, may be a trispecific antibody or a trispecific antigen binding fragment comprising:
(ii) an antigen-binding site comprising (a) the CDRH1, CDRH2, CDRH3, CDRL1, CDRL2 and CDRL3 sequences and/or (b) the VH and VL sequences of 15E3, or sequence variants thereof, as described above;
(iii) an antigen-binding site comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antigen-binding site comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the multispecific antibody, or the multispecific antigen binding fragment, may be a trispecific antibody or a trispecific antigen binding fragment comprising:
(i) an antigen-binding site comprising (a) the CDRH1, CDRH2, CDRH3, CDRL1, CDRL2 and CDRL3 sequences and/or (b) the VH and VL sequences of 17H9, or sequence variants thereof, as described above;
(iii) an antigen-binding site comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antigen-binding site comprising (a) the CDRH1, CDRH2, CDRH3, CDRL1, CDRL2 and CDRL3 sequences and/or (b) the VH and VL sequences of 7G6, or sequence variants thereof, as described above.

Preferably, the multispecific antibody, or the multispecific antigen binding fragment, may be a trispecific antibody or a trispecific antigen binding fragment comprising:
(i) an antigen-binding site comprising (a) the CDRH1, CDRH2, CDRH3, CDRL1, CDRL2 and CDRL3 sequences and/or (b) the VH and VL sequences of 17H9, or sequence variants thereof, as described above;
(ii) an antigen-binding site comprising (a) the CDRH1, CDRH2, CDRH3, CDRL1, CDRL2 and CDRL3 sequences and/or (b) the VH and VL sequences of 15E3, or sequence variants thereof, as described above; and
(iv) an antigen-binding site comprising (a) the CDRH1, CDRH2, CDRH3, CDRL1, CDRL2 and CDRL3 sequences and/or (b) the VH and VL sequences of 7G6, or sequence variants thereof, as described above.

It is also preferred that the multispecific antibody, or the multispecific antigen binding fragment, may be a tetraspecific antibody or a tetraspecific antigen binding fragment comprising:
(i) an antigen-binding site comprising (a) the CDRH1, CDRH2, CDRH3, CDRL1, CDRL2 and CDRL3 sequences and/or (b) the VH and VL sequences of 17H9, or sequence variants thereof, as described above;
(ii) an antigen-binding site comprising (a) the CDRH1, CDRH2, CDRH3, CDRL1, CDRL2 and CDRL3 sequences and/or (b) the VH and VL sequences of 15E3, or sequence variants thereof, as described above;
(iii) an antigen-binding site comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antigen-binding site comprising (a) the CDRH1, CDRH2, CDRH3, CDRL1, CDRL2 and CDRL3 sequences and/or (b) the VH and VL sequences of 7G6, or sequence variants thereof, as described above.

The present invention also provides a multispecific antibody, or a multispecific antigen-binding fragment, which binds to distinct, non-overlapping epitopes of Ara h 2, wherein the antibody, or the antigen-binding fragment, comprises at least two of the following:
(i) an antigen-binding site comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
(ii) an antigen-binding site comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
(iii) an antigen-binding site comprising a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22; and/or
(iv) an antigen-binding site comprising a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30.

Preferably, the multispecific antibody, or the multispecific antigen binding fragment, comprises at least two of the following:
(i) an antigen-binding site comprising a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antigen-binding site comprising a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
(iii) an antigen-binding site comprising a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22; and/or
(iv) an antigen-binding site comprising a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30.

More preferably, the multispecific antibody, or the multispecific antigen binding fragment, comprises at least two of the following:
(i) an antigen-binding site comprising a VH having at least 70% identity to any one of SEQ ID NOs 7, 44 and 45; and a VL having at least 70% identity to any one of SEQ ID NOS 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
(ii) an antigen-binding site comprising a VH having at least 70% identity to any one of SEQ ID NOs 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
(iii) an antigen-binding site comprising a VH having at least 70% identity to any one of SEQ ID NOs 23, 41, 50 and 53; and a VL having at least 70% identity to any one of SEQ ID NOs 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained; and/or
(iv) an antigen-binding site comprising a VH having at least 70% identity to any one of SEQ ID NOs 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained.

Even more preferably, the multispecific antibody, or the multispecific antigen binding fragment, comprises at least two of the following:
(i) an antigen-binding site comprising a VH according to any one of SEQ ID NOs 7, 44 and 45; and a VL according to any one of SEQ ID NOs 8, 46, 47, 48 and 49;
(ii) an antigen-binding site comprising a VH according to any one of SEQ ID NOs 15, 37 and 38; and a VL according to any one of SEQ ID NOs 16, 39 and 40;
(iii) an antigen-binding site comprising a VH according to any one of SEQ ID NOs 23, 41, 50 and 53; and a VL according to any one of SEQ ID NOs 24, 42 and 43; and/or
(iv) an antigen-binding site comprising a VH according to any one of SEQ ID NOs 31, 33 and 34; and a VL according to any one of SEQ ID NOs 32, 35, 36 and 54.

In some embodiments, the multispecific antibody, or the multispecific antigen binding fragment, may be a bispecific antibody or a bispecific antigen binding fragment comprising:
(i) an antigen-binding site comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49; and
(ii) an antigen-binding site comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40.

In some embodiments, the multispecific antibody, or the multispecific antigen binding fragment, may be a bispecific antibody or a bispecific antigen binding fragment comprising: an antigen-binding site comprising:
(i) an antigen-binding site comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49; and
(iii) an antigen-binding site comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43.

In some embodiments, the multispecific antibody, or the multispecific antigen binding fragment, may be a bispecific antibody or a bispecific antigen binding fragment comprising:
(ii) an antigen-binding site comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40; and
(iv) an antigen-binding site comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

In some embodiments, the multispecific antibody, or the multispecific antigen binding fragment, may be a bispecific antibody or a bispecific antigen binding fragment comprising:
(iii) an antigen-binding site comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antigen-binding site comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

Preferably, the multispecific antibody, or the multispecific antigen binding fragment, may be a bispecific antibody or a bispecific antigen binding fragment comprising:
(ii) an antigen-binding site comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40; and
(iii) an antigen-binding site comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43.

More preferably, the multispecific antibody, or the multispecific antigen binding fragment, may be a bispecific antibody or a bispecific antigen binding fragment comprising:
(i) an antigen-binding site comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49; and
(iv) an antigen-binding site comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

In some embodiments, the multispecific antibody, or the multispecific antigen binding fragment, may be a trispecific antibody or a trispecific antigen binding fragment comprising:
(i) an antigen-binding site comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(ii) an antigen-binding site comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40; and
(iii) an antigen-binding site comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43.

In some embodiments, the multispecific antibody, or the multispecific antigen binding fragment, may be a trispecific antibody or a trispecific antigen binding fragment comprising:
(i) an antigen-binding site comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(iii) an antigen-binding site comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antigen-binding site comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOs 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

In some embodiments, the multispecific antibody, or the multispecific antigen binding fragment, may be a trispecific antibody or a trispecific antigen binding fragment comprising:
(ii) an antigen-binding site comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40;
(iii) an antigen-binding site comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antigen-binding site comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

Preferably, the multispecific antibody, or the multispecific antigen binding fragment, may be a trispecific antibody or a trispecific antigen binding fragment comprising:
(i) an antigen-binding site comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOs 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(ii) an antigen-binding site comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40; and
(iv) an antigen-binding site comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

It is also preferred that the multispecific antibody, or the multispecific antigen binding fragment, may be a tetraspecific antibody or a tetraspecific antigen binding fragment comprising:
(i) an antigen-binding site comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(ii) an antigen-binding site comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40;
(iii) an antigen-binding site comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antigen-binding site comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

More preferably, the multispecific antibody, or the multispecific antigen binding fragment, may be a tetraspecific antibody or a tetraspecific antigen binding fragment comprising:
(i) an antigen-binding site comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6; in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antigen-binding site comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14; in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
(iii) an antigen-binding site comprising a CDRH1 having at least 70% identity to according to SEQ ID NO: 17, a CDRH2 having at least 70% identity to SEQ ID NO: 18, a CDRH3 having at least 70% identity to SEQ ID NO: 51, a CDRL1 having at least 70% identity to SEQ ID NO: 20, a CDRL2 having at least 70% identity to SEQ ID NO: 21, and a CDRL3 having at least 70% identity to SEQ ID NO: 22; in particular a CDRH1 according to according to SEQ ID NO: 17, a CDRH2 according to SEQ ID NO: 18, a CDRH3 according to SEQ ID NO: 51, a CDRL1 according to SEQ ID NO: 20, a CDRL2 according to SEQ ID NO: 21, and a CDRL3 according to SEQ ID NO: 22; and
(iv) an antigen-binding site comprising a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30; in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30.

Even more preferably, the multispecific antibody, or the multispecific antigen binding fragment, may be a tetraspecific antibody or a tetraspecific antigen binding fragment comprising:
(i) an antigen-binding site comprising a VH having at least 70% identity to SEQ ID NO: 45; and a VL having at least 70% identity to SEQ ID NO: 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained; such as a VH according to SEQ ID NO: 45, and a VL according to SEQ ID NO: 49;
(ii) an antigen-binding site comprising a VH having at least 70% identity to SEQ ID NO: 38; and a VL having at least 70% identity to SEQ ID NO: 39; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained; such as a VH according to SEQ ID NO: 38, and a VL according to SEQ ID NO: 39;
(iii) an antigen-binding site comprising a VH having at least 70% identity to SEQ ID NO: 53; and a VL having at least 70% identity to SEQ ID NO: 43; wherein the CDRH1 according to according to SEQ ID NO: 17, CDRH2 according to SEQ ID NO: 18, CDRH3 according to SEQ ID NO: 51, CDRL1 according to SEQ ID NO: 20, CDRL2 according to SEQ ID NO: 21, and CDRL3 according to SEQ ID NO: 22 are preferably maintained; such as a VH according to SEQ ID NO: 53, and a VL according to SEQ ID NO: 43; and
(iv) an antigen-binding site comprising a VH having at least 70% identity to SEQ ID NO: 34; and a VL having at least 70% identity to SEQ ID NO: 35 or 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained; such as a VH according to SEQ ID NO: 34, and a VL according to SEQ ID NO: 35 or 54.

In other words, the antigen-binding sites (paratopes) of the multispecific antibody, or the multispecific antigen binding fragment, may be selected from the four different antibodies 17H9, 15E3, 2F8 and 7G6, or the variants thereof, as described above, in particular as shown in Tables 1 and 2.

In some embodiments, the multispecific antibody, or the multispecific antigen binding fragment, is bispecific and comprises two distinct antigen-binding sites selected from (i)-(iv).

Such a bispecific antibody may be combined, in use, with a second antibody comprising one or both of the remaining antigen-binding sites selected from (i)-(iv) (i.e. with one or both of those binding antigen-binding sites selected from (i)-(iv), which are not contained in the (first) bispecific antibody). For example, two bispecific antibodies, each containing two distinct antigen-binding sites selected from (i)-(iv) may be combined, such that the combination of the two bispecific antibodies includes all four antigen-binding sites of (i) to (iv). Likewise, the bispecific antibody may be combined with one or two monospecific antibodies including one of the remaining antigen-binding sites. For combination, the antibodies may be comprised in the same composition or in different compositions (but administered in a combined treatment schedule).

Preferably, the multispecific antibody, or the multispecific antigen binding fragment, is trispecific and comprises three distinct antigen-binding sites selected from (i)-(iv).

It is also preferred that the multispecific antibody, or the multispecific antigen binding fragment, is tetraspecific and comprises the four distinct antigen-binding sites according to (i), (ii), (iii) and (iv).

### Nucleic Acids

In another aspect, the invention also provides a nucleic acid molecule comprising a polynucleotide encoding the antibody according to the present invention, or an antigen-binding fragment thereof, as described above.

In some embodiments, the nucleic acid molecule comprises one or more polynucleotide(s) encoding the exemplified antibodies of the invention (e.g., as described above, in particular in Tables 1 and 2), or a sequence variant thereof as described herein (e.g., having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity as described above).

Exemplified nucleic acid sequences encoding the CDR and VH/VL sequences of exemplified antibodies as described herein are shown in Table 3 below.

TABLE 3
SEQ ID NOs for polynucleotide sequences encoding the CDR and VH/VL sequences
of fully human antibodies 17H9, 15E3, 2F8 and 7G6. For 2F8, SEQ ID NO:
82 encodes the wildtype CDRH3, while SEQ ID NO: 88 encodes the variant
CDRH3. The respective VH are encoded by SEQ ID NO: 86 (wildtype VH) and
SEQ ID NO: 89 (variant VH). For 7G6, SEQ ID NO: 94 encodes the wildtype
CDRL2, while SEQ ID NO: 98 encodes the variant CDRL2. The respective VL
are encoded by SEQ ID NO: 97 (wildtype VL) and SEQ ID NO: 99 (variant VL).

| Antibody | CDRH1 | CDRH2 | CDRH3 | CDRL1 | CDRL2 | CDRL3 | VH | VL |
|---|---|---|---|---|---|---|---|---|
| 17H9 | 64 | 65 | 66 | 67 | 68 | 69 | 70 | 71 |
| 15E3 | 72 | 73 | 74 | 75 | 76 | 77 | 78 | 79 |
| 2F8 | 80 | 81 | 82/88 | 83 | 84 | 85 | 86/89 | 87 |
| 7G6 | 90 | 91 | 92 | 93 | 94/98 | 95 | 96 | 97/99 |

Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA. Nucleic acids may encode the light chain and/or the heavy chain of an antibody (or a single chain antibody). In other words, the light chain and the heavy chain of the antibody may be encoded by the same nucleic acid molecule (e.g., for single chain antibodies or for antibodies with separate heavy and light chains in bicistronic manner or an expression cassette containing more than one ribosome entry site such as IRES). Alternatively, the light chain and the heavy chain of the antibody may be encoded by distinct nucleic acid molecules. In a similar manner, for multispecific antibodies comprising two or more immunoglobulin chains, the different chains may be encoded by the same nucleic acid molecule (e.g., in a multicistronic manner or an expression cassette containing more than one ribosome entry site such as IRES). Alternatively, the different immunoglobulin chains of the multispecific antibody may be encoded by distinct nucleic acid molecules.

Due to the redundancy of the genetic code, the present invention also comprises sequence variants of nucleic acid sequences, which encode the same amino acid sequences. The polynucleotide encoding the antibody (or the complete nucleic acid molecule) may be optimized for expression of the antibody. For example, codon optimization of the nucleotide sequence may be used to improve the efficiency of translation in expression systems for the production of the antibody. Moreover, the nucleic acid molecule may comprise heterologous elements (i.e., elements, which in nature do not occur on the same nucleic acid molecule as the coding sequence for the (heavy or light chain of) an antibody. For example, a nucleic acid molecule may comprise a heterologous promotor, a heterologous enhancer, heterologous UTR (e.g., for optimal translation/expression), a heterologous poly-A-tail, heterologous DNA insulator elements and the like.

A nucleic acid molecule is a molecule comprising nucleic acid components. The term nucleic acid molecule usually refers to DNA or RNA molecules. It may be used synonymous with the term “polynucleotide”, i.e. the nucleic acid molecule may consist of a polynucleotide encoding the antibody. Alternatively, the nucleic acid molecule may also comprise further elements in addition to the polynucleotide encoding the antibody. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

In general, the nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g., labels) or can introduce tags (e.g., for purification purposes). Alternatively, a mutation in a nucleic acid sequence may be “silent”, i.e. not reflected in the amino acid sequence due to the redundancy of the genetic code. In general, mutations can be introduced in specific sites or can be introduced at random, followed by selection (e.g., molecular evolution). For instance, one or more nucleic acids encoding any of the light or heavy chains of an (exemplary) antibody can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the polynucleotide encoding the antibody, or an antigen-binding fragment thereof, (or the (complete) nucleic acid molecule) may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel D C, Jahn D, JCat: a novel tool to adapt codon usage of a target gene to its potential expression host. Nucleic Acids Res. 2005 Jul. 1; 33 (Web Server issue): W526-31; or, for example, Genscript's OptimumGene™ algorithm (as described in US 2011/0081708 A1).

For example, the nucleic acid molecule of the invention may comprise a nucleic acid sequence as set forth in any one of SEQ ID NOs 64-99; or a sequence variant thereof having at least 70%, at least 75%, at least 80%, at least 85%, at least 88%, at least 90%, at least 92%, at least 95%, at least 96%, at least 97%, at least 98% or at least 99% sequence identity. Thereby, the nucleic acid molecule may encode any one of the exemplified antibodies 17H9, 15E3, 2F8 and 7G6 (by combining the sequences as shown in Table 3), or a sequence variant thereof as described herein.

The present invention also provides a plurality of nucleic acid molecules encoding the antibody, or an antigen-binding fragment thereof, as described herein, wherein each of the nucleic acid molecules (of the plurality of nucleic acid molecules) comprises a polynucleotide encoding an immunoglobulin chain of the antibody, or an antigen-binding fragment thereof. Thereby, the plurality of nucleic acid molecules, taken together, encodes (all of the immunoglobulin chains of) the antibody, or an antigen-binding fragment thereof, as described herein. In some embodiments, the plurality of nucleic acid molecules encoding the antibody, or an antigen-binding fragment thereof, as described herein, may be a combination of a first and a second nucleic acid molecule, wherein the first nucleic acid molecule comprises a polynucleotide encoding the heavy chain of the antibody, or an antigen-binding fragment thereof, of the present invention; and the second nucleic acid molecule comprises a polynucleotide encoding the corresponding light chain of the same antibody, or the same antigen-binding fragment thereof.

In general, the above description regarding the (general) features of the nucleic acid molecule of the invention applies accordingly to the nucleic acid molecules of the plurality of nucleic acid molecules. Accordingly, one or more of the polynucleotides encoding the immunoglobulin chains of the antibody, or an antigen-binding fragment thereof, may be codon-optimized. For example, the plurality may comprise a nucleic acid sequence as set forth in any one of SEQ ID NOs 64-99; or a sequence variant thereof having at least 70%, at least 75%, at least 80%, at least 85%, at least 88%, at least 90%, at least 92%, at least 95%, at least 96%, at least 97%, at least 98% or at least 99% sequence identity. Thereby, the plurality of nucleic acid molecules may encode any one of the exemplified antibodies 17H9, 15E3, 2F8 and 7G6 (by combining the sequences as shown in Table 3), or a sequence variant thereof as described herein.

### Vectors

Further included within the scope of the invention are vectors, for example, expression vectors, comprising a nucleic acid molecule according to the present invention or the plurality of nucleic acid molecules according to the present invention. Usually, a vector comprises a nucleic acid molecule as described above.

The present invention also provides a plurality of vectors comprising the plurality of nucleic acid molecules according to invention as described above. Thereby, each vector of the plurality of vectors may contain one or more nucleic acid molecules of the plurality of nucleic acid molecules according to invention as described above. In some embodiments, the plurality of vectors may be a combination of a first and a second vector, wherein the first vector comprises a first nucleic acid molecule as described above (for the combination of nucleic acid molecules) and the second vector comprises a second nucleic acid molecule as described above (for the combination of nucleic acid molecules).

A vector is usually a recombinant nucleic acid molecule, i.e. a nucleic acid molecule which does not occur in nature. Accordingly, the vector may comprise heterologous elements (i.e., sequence elements of different origin in nature). For example, the vector may comprise a multiple cloning site, a heterologous promotor, a heterologous enhancer, a heterologous selection marker (to identify cells comprising said vector in comparison to cells not comprising said vector), heterologous origin of replications, heterologous DNA insulator elements and the like. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a (heavy and/or light chain of a) desired antibody according to the present invention. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. For example, a vector in the sense of the present application comprises a cloning site, a selection marker, such as an antibiotic resistance factor, and a sequence suitable for multiplication of the vector, such as an origin of replication. A vector in the context of the present application may be a plasmid vector.

As used herein, the term “vector” may also refer to a delivery vector, e.g. for viral or non-viral delivery of a nucleic acid of the invention. Alternatively, it may be referred to viral or non-viral delivery systems. Accordingly, the present invention also provides a delivery vector/system comprising the nucleic acid molecule as described above (or comprising an expression vector as described above). The delivery vector/system may be viral or non-viral. Various examples of viral and non-viral delivery vectors/systems are known in the art and described, for example, in Nayerossadat N, Maedeh T, Ali P A. Viral and nonviral delivery systems for gene delivery. Adv Biomed Res. 2012; 1:27. doi: 10.4103/2277-9175.98152, which is incorporated herein by reference. Non-limiting examples of viral delivery vectors/systems include retroviral vectors; adenoviral vectors; adeno-associated viral (AAV) vectors, including helper-dependent adenoviral vectors and hybrid adenoviral vectors; herpes simplex virus vectors; lentivirus vectors; poxvirus vectors and Epstein-Barr virus vectors.

Among the viral vectors, adenoviral vectors and adeno-associated viral (AAV) vectors are preferred. Non-limiting examples of non-viral delivery vectors/systems include chemical and non-chemical methods. Non-chemical delivery includes physical methods, such as electroporation and other methods for transient penetration of the cell membrane by mechanical, electrical, ultrasonic, hydrodynamic, or laser-based energy; naked DNA or RNA delivery; gene gun; hydrodynamic delivery; ultrasound delivery and magnetofection. Chemical non-viral delivery systems include cationic particles, in particular cationic lipids/liposomes, cationic polymers and lipid/polymer systems. Among non-viral vectors/systems, cationic liposomes are preferred.

### Cells

In a further aspect, the present invention also provides a (host) cell expressing the antibody according to the present invention, or an antigen-binding fragment thereof; and/or comprising the vector (or the plurality of vectors) according the present invention. The (host) cell may be an isolated cell, which is not part of a human or animal body, e.g. a cell line or an engineered cell. The cell may express the nucleic acid(s) or vector(s) of the invention in a recombinant manner, e.g. in a heterologous manner (i.e., the cell/cell type does not express the antibody or the antigen-binding fragment in nature).

Examples of such cells include, but are not limited to, eukaryotic cells, e.g., yeast cells, animal cells or plant cells. Other examples of such cells include, but are not limited to, prokaryotic cells, e.g. E. coli. In some embodiments, the cells are mammalian cells, such as a mammalian cell line. Examples include human cells, CHO cells, HEK293 cells, PER.C6 cells, NSO cells, human liver cells, myeloma cells or hybridoma cells.

The cell may be transfected with a vector according to the present invention, for example with an expression vector. The term “transfection” refers to the introduction of nucleic acid molecules, such as DNA or RNA (e.g. mRNA) molecules, into cells, e.g. into eukaryotic or prokaryotic cells. In the context of the present invention, the term “transfection” encompasses any method known to the skilled person for introducing nucleic acid molecules into cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral.

Moreover, the cells of the present invention may be transfected stably or transiently with the vector according to the present invention, e.g. for expressing the antibody according to the present invention. In some embodiments, the cells are stably transfected with the vector according to the present invention encoding the antibody according to the present invention. In other embodiments, the cells are transiently transfected with the vector according to the present invention encoding the antibody according to the present invention.

Accordingly, the present invention also provides a recombinant host cell, which heterologously expresses the antibody of the invention or the antigen-binding fragment thereof. For example, the cell may be of another species than the antibody (e.g., CHO cells expressing human antibodies). In some embodiments, the cell type of the cell does not express (such) antibodies in nature. Moreover, the host cell may impart a post-translational modification (PTM; e.g., glycosylation) on the antibody that is not present in their native state. Such a PTM may result in a functional difference (e.g., decreased immunogenicity). Accordingly, the antibody of the invention, or the antigen-binding fragment thereof, may have a post-translational modification, which is distinct from the naturally produced antibody (e.g., an antibody of an immune response in a human).

### Production of Antibodies

Antibodies according to the invention can be made by any method known in the art. For example, the general methodology for making monoclonal antibodies using hybridoma technology is well known (Kohler, G. and Milstein, C., 1975; Kozbar et al. 1983).

Standard techniques of molecular biology may be used to prepare DNA sequences encoding the antibodies or antigen-binding fragments of the present invention. Desired DNA sequences may be synthesized completely or in part, e.g., using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention. Eukaryotic, e.g., mammalian, host cell expression systems may be used for production of antibody molecules, such as complete antibody molecules. Suitable mammalian host cells include, but are not limited to, CHO, HEK293, PER.C6, NSO, myeloma or hybridoma cells. Also, prokaryotic, e.g. bacterial host cell expression systems may be used for the production of antibody molecules, such as complete antibody molecules. Suitable bacterial host cells include, but are not limited to, E. coli cells.

Accordingly, the present invention provides a method for preparing the antibody, or an antigen-binding fragment or an immunoglobulin chain(s) thereof, according to the present invention, said method comprising
(i) culturing the host cell as described above; and
(ii) isolating the antibody or immunoglobulin chain(s) thereof from the culture.

In other words, the present invention also provides a process for the production of an antibody molecule according to the present invention comprising culturing a (heterologous) host cell comprising a vector encoding a nucleic acid of the present invention, in particular under conditions suitable for expression of protein from DNA encoding the antibody molecule of the present invention, and isolating the antibody molecule.

For production of the antibody comprising both heavy and light chains, a host cell, such as a cell line, may be transfected with two vectors, a first vector encoding a light chain polypeptide and a second vector encoding a heavy chain polypeptide, e.g. as described above. Alternatively, a single vector may be used, the vector including sequences encoding light chain and heavy chain polypeptides (e.g. for single chain antibodies or in a bicistronic manner). Likewise, a plurality of vectors may be used, if multispecific antibodies with two or more immunoglobulin chains shall be expressed.

Thus, the invention also provides a method for preparing a recombinant cell, comprising the steps of: (i) providing one or more nucleic acids that encode(s) the antibody of the invention; (ii) inserting the nucleic acid into an expression vector and (iii) transfecting the vector into a (heterologous) host cell in order to permit expression of the antibody of interest in that host cell. The nucleic acid of step (i) may, but need not, be manipulated to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest. Thus the procedures for first preparing the nucleic acid(s) and then using it to transfect a host cell can be performed at different times by different people in different places (e.g., in different countries).

These recombinant cells of the invention can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

The transfected host cell may be a eukaryotic cell, including yeast and animal cells, particularly mammalian cells (e.g., CHO cells, NSO cells, human cells such as PER.C6, HEK293 or HKB-11 cells, myeloma cells, or a human liver cell), as well as plant cells. In some embodiments, the transfected host cell is a mammalian cell, such as a human cell. In some embodiments, expression hosts can glycosylate the antibody of the invention, particularly with carbohydrate structures that are not themselves immunogenic in humans. In some embodiments the transfected host cell may be able to grow in serum-free media. In further embodiments the transfected host cell may be able to grow in culture without the presence of animal-derived products. The transfected host cell may also be cultured to give a cell line.

The invention also provides a method of preparing the antibody of interest comprising the steps of: culturing or sub-culturing a transfected host cell population, e.g. a stably transfected host cell population, under conditions where the antibody of interest is expressed and, optionally, purifying the antibody of interest. The transfected host cell population may be prepared by (i) providing nucleic acid(s) encoding a selected antibody of interest, (ii) inserting the nucleic acid(s) into an expression vector, (iii) transfecting the vector in a host cell that can express the antibody of interest, and (iv) culturing or sub-culturing the transfected host cell comprising the inserted nucleic acids to produce the antibody of interest.

In some embodiments, antibodies according to the invention may be produced by (i) expressing a nucleic acid sequence according to the invention in a host cell, e.g. by use of a vector (or host cell) according to the present invention, and (ii) isolating the expressed antibody product. Additionally, the method may include (iii) purifying the isolated antibody.

Accordingly, after production, the antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

### Compositions and Kits

The present invention also provides a composition comprising one or more of:
(i) the antibody of the present invention, or an antigen-binding fragment thereof;
(ii) the nucleic acid or the plurality of nucleic acids of the present invention;
(iii) the vector or the plurality of vectors of the present invention; or
(iv) the cell expressing the antibody according to the present invention or comprising the vector according to the present invention.

The composition may be used for treatment or diagnostic purposes. Accordingly, the composition may be a pharmaceutical composition or a diagnostic composition. The composition may comprise a (pharmaceutically acceptable) excipient, diluent or carrier.

Accordingly, the present invention also provides a pharmaceutical composition comprising the antibody according to the present invention, or an antigen-binding fragment thereof, the nucleic acid or the plurality of nucleic acids of the present invention, the vector or the plurality of vectors of the present invention, and/or the cell according to the present invention.

The pharmaceutical composition may optionally also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In some embodiments, the pharmaceutically acceptable carrier, diluent and/or excipient in the pharmaceutical composition is not an active component in respect to peanut allergy.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin®, for reconstitution with sterile water containing a preservative). The composition may be prepared for topical administration e.g., as an ointment, cream or powder. The composition may be prepared for oral administration e.g., as a tablet or capsule, as a spray, or as a syrup (optionally flavored). The composition may be prepared for pulmonary administration e.g., as an inhaler, using a fine powder or a spray. The composition may be prepared as a suppository or pessary. The composition may be prepared for nasal, aural or ocular administration e.g., as drops. The composition may be in kit form, designed such that a combined composition is reconstituted just prior to administration to a subject. For example, a lyophilized antibody may be provided in kit form with sterile water or a sterile buffer.

In some embodiments, the (only) active ingredient in the composition is the antibody as described herein. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

The present invention also provides a method of preparing a pharmaceutical composition comprising the steps of: (i) preparing an antibody of the invention; and (ii) admixing the purified antibody with one or more pharmaceutically acceptable excipients, diluents or carriers.

In other embodiments, a method of preparing a pharmaceutical composition comprises the step of: admixing an antibody with one or more pharmaceutically-acceptable carriers, wherein the antibody is a monoclonal antibody.

Pharmaceutical compositions may generally have a pH between 5.5 and 8.5, in some embodiments this may be between 6 and 8, for example about 7. The pH may be maintained by the use of a buffer. The composition may be sterile and/or pyrogen free. The composition may be isotonic with respect to humans. In some embodiments pharmaceutical compositions are supplied in hermetically-sealed containers.

Within the scope of the invention are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody may be in dry form, for reconstitution before use with an appropriate sterile liquid.

A vehicle is typically understood to be a material that is suitable for storing, transporting, and/or administering a compound, such as a pharmaceutically active compound, in particular the antibodies as described herein. For example, the vehicle may be a physiologically acceptable liquid, which is suitable for storing, transporting, and/or administering a pharmaceutically active compound, in particular the antibodies as described herein. Once formulated, the compositions can be administered directly to the subject. In some embodiments the compositions are adapted for administration to mammalian, e.g., human subjects.

Pharmaceutical compositions may include an antimicrobial, particularly if packaged in a multiple dose format. They may comprise detergent e.g., a Tween (polysorbate), such as Tween 80. Detergents are generally present at low levels e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

The pharmaceutical compositions may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Optionally, the pharmaceutical composition may be prepared for oral administration, e.g. as tablets, capsules and the like, for topical administration, or as injectable, e.g. as liquid solutions or suspensions. In some embodiments, the pharmaceutical composition is an injectable. Solid forms suitable for solution in, or suspension in, liquid vehicles prior to injection are also encompassed, for example the pharmaceutical composition may be in lyophilized form.

For injection, e.g. intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is an antibody, a peptide, a nucleic acid molecule, or another pharmaceutically useful compound that is to be given to an individual, administration is usually in an “effective amount”, e.g. in a “prophylactically effective amount” or a “therapeutically effective amount” (as the case may be), this being sufficient to show benefit to the individual. The actual amount administered, and rate and time-course of administration, will depend on the nature and severity of what is being treated. For injection, the pharmaceutical composition may be provided for example in a pre-filled syringe.

The pharmaceutical composition may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the antibody as defined above, is combined with emulsifying and suspending agents. If desired, certain sweetening, flavoring or coloring agents may also be added.

The pharmaceutical composition may also be administered topically, especially when the target of treatment includes areas or organs readily accessible by topical application, e.g. including accessible epithelial tissue. Suitable topical formulations are readily prepared for each of these areas or organs. For topical applications, the pharmaceutical composition may be formulated in a suitable ointment, containing the pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the pharmaceutical composition can be formulated in a suitable lotion or cream. Suitable carriers include, but are not limited to, mineral oil, sorbitan monostearate, polysorbate 60, cetyl esters wax, cetearyl alcohol, 2-octyldodecanol, benzyl alcohol and water.

Dosage treatment may be a single dose schedule or a multiple dose schedule. For a single dose, e.g. a daily, weekly or monthly dose, the amount of the antibody in the pharmaceutical composition, may not exceed 1 g or 500 mg. In some embodiments, for a single dose, the amount of the antibody in the pharmaceutical composition, may not exceed 200 mg, or 100 mg. For example, for a single dose, the amount of the antibody in the pharmaceutical composition, may not exceed 50 mg.

In some embodiments, the composition may include antibodies of the invention, wherein the antibodies may make up at least 50% by weight (e.g., 60%, 70%, 75%, 80%, 85%, 90%, 95%, 96%, 97%, 98%, 99% or more) of the total protein in the composition. In the composition, the antibodies may be in purified form.

As an alternative to delivering antibodies for therapeutic purposes, it is possible to deliver nucleic acid (typically DNA) that encodes the monoclonal antibody of interest to a subject, such that the nucleic acid can be expressed in the subject in situ to provide a desired therapeutic effect. Suitable gene therapy and nucleic acid delivery vectors are known in the art.

Pharmaceutical compositions typically include an “effective” amount of one or more antibodies as described herein, i.e. an amount that is sufficient to treat, ameliorate, attenuate, decrease or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician. An effective dose may generally be from about 0.005 to about 100 mg/kg, for example from about 0.0075 to about 50 mg/kg or from about 0.01 to about 10 mg/kg. In some embodiments, the effective dose will be from about 0.02 to about 5 mg/kg, of the antibody (e.g. amount of the antibody in the pharmaceutical composition) in relation to the bodyweight (e.g., in kg) of the individual to which it is administered.

Moreover, the pharmaceutical composition may also comprise an additional active component, which may be a further antibody or a component, which is not an antibody. In other embodiments, the pharmaceutical composition may not comprise an additional active component (in addition to the antibody of the invention or respective nucleic acids, vectors or cells as described above).

Accordingly, the pharmaceutical composition may comprise one or more of the additional active components. The antibody of the invention can be present either in the same pharmaceutical composition as the additional active component or, alternatively, the antibody may be comprised by a first pharmaceutical composition and the additional active component may be comprised by a second pharmaceutical composition different from the first pharmaceutical composition. Accordingly, if more than one additional active component is envisaged, each additional active component and the antibody may be comprised in a different pharmaceutical composition. Such different pharmaceutical compositions may be administered either combined/simultaneously or at separate times or at separate locations (e.g. separate parts of the body), optionally by different routes of administration.

The antibody and the additional active component may provide an additive therapeutic effect, such as a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

Preferably, the composition comprises at least two distinct antibodies, or antigen-binding fragments thereof, binding to distinct, non-overlapping epitopes of Ara h 2. More preferably, the composition comprises (exactly) three distinct antibodies, or antigen-binding fragments thereof, binding to distinct, non-overlapping epitopes of Ara h 2. Even more preferably, the composition comprises (exactly) four distinct antibodies, or antigen-binding fragments thereof, binding to distinct, non-overlapping epitopes of Ara h 2. To this end, the antibodies contained in the composition may be selected from the four different antibodies 17H9, 15E3, 2F8 and 7G6, or the variants thereof, as described above, e.g. in Tables 1 and 2. As shown in the appended examples, 17H9, 15E3, 2F8 and 7G6 bind to distinct, non-overlapping epitopes of Ara h 2.

Accordingly, the composition may comprise at least two of the following:
(i) the antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above;
(ii) the antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above;
(iii) the antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and/or
(iv) the antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the composition comprises (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above.

In some embodiments, the composition comprises (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and
(iii) an antibody, or an antigen-binding fragment thereof, comprising (a) the CDRH1, sequences of 2F8, or sequence variants thereof, as described above.

In some embodiments, the composition comprises (exactly) two distinct antibodies, namely:
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the composition comprises (exactly) two distinct antibodies, namely:
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

Preferably, the composition comprises (exactly) two distinct antibodies, namely:
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above.

More preferably, the composition comprises (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and
(iv) an antibody, or an antigen-binding fragment thereof, comprising (a) the CDRH1, sequences of 7G6, or sequence variants thereof, as described above.

Preferably, the composition comprises (exactly) three or four distinct antibodies, or antigen-binding fragments, binding to distinct, non-overlapping epitopes of Ara h 2, wherein the (exactly) three or four distinct antibodies, or antigen-binding fragments, are preferably selected from (i)-(iv) above.

In some embodiments, the composition comprises (exactly) three distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above.

In some embodiments, the composition comprises (exactly) three distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above;
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the composition comprises (exactly) three distinct antibodies, namely:
(ii) an antibody, or an antigen-binding fragment thereof, comprising (a) the CDRH1, sequences of 15E3, or sequence variants thereof, as described above;
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

Preferably, the composition comprises (exactly) three distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

It is also preferred that the composition comprises (exactly) four distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above;
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antibody, or an antigen-binding fragment thereof, comprising (a) the CDRH1, sequences of 7G6, or sequence variants thereof, as described above.

In some embodiments, the composition may comprise (i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and (ii) an antibody, or an antigen-binding fragment thereof, comprising (a) the CDRH1, CDRH2, CDRH3, CDRL1, CDRL2 and CDRL3 sequences and/or (b) the VH and VL sequences of (wild-type) 7G6, or sequence variants thereof, as described herein, e.g. in Tables 1 and 2. Optionally, such a composition may further comprise an antibody, or an antigen-binding fragment thereof, including the CDR and/or VH/VL sequences of 15E3 (or a sequence variant thereof), and/or an antibody, or an antigen-binding fragment thereof, including the CDR and/or VH/VL sequences of 2F8 (or a sequence variant thereof).

In some embodiments, the composition may comprise (i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and (ii) an antibody, or an antigen-binding fragment thereof, comprising (a) the CDRH1, CDRH2, CDRH3, CDRL1, CDRL2 and CDRL3 sequences and/or (b) the VH and VL sequences of (wild-type) 2F8, or sequence variants thereof, as described herein, e.g. in Tables 1 and 2. Optionally, such a composition may further comprise an antibody, or an antigen-binding fragment thereof, including the CDR and/or VH/VL sequences of 15E3 (or a sequence variant thereof), and/or an antibody, or an antigen-binding fragment thereof, including the CDR and/or VH/VL sequences of 7G6 (or a sequence variant thereof).

In some embodiments, the composition may comprise (i) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and (ii) an antibody, or an antigen-binding fragment thereof, comprising (a) the CDRH1, CDRH2, CDRH3, CDRL1, CDRL2 and CDRL3 sequences and/or (b) the VH and VL sequences of (wild-type) 7G6, or sequence variants thereof, as described herein, e.g. in Tables 1 and 2. Optionally, such a composition may further comprise an antibody, or an antigen-binding fragment thereof, including the CDR and/or VH/VL sequences of 17H9 (or a sequence variant thereof), and/or an antibody, or an antigen-binding fragment thereof, including the CDR and/or VH/VL sequences of 2F8 (or a sequence variant thereof).

In some embodiments, the composition may comprise (i) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and (ii) an antibody, or an antigen-binding fragment thereof, comprising (a) the CDRH1, CDRH2, CDRH3, CDRL1, CDRL2 and CDRL3 sequences and/or (b) the VH and VL sequences of (wild-type) 2F8, or sequence variants thereof, as described herein, e.g. in Tables 1 and 2. Optionally, such a composition may further comprise an antibody, or an antigen-binding fragment thereof, including the CDR and/or VH/VL sequences of 17H9 (or a sequence variant thereof), and/or an antibody, or an antigen-binding fragment thereof, including the CDR and/or VH/VL sequences of 7G6 (or a sequence variant thereof).

The present invention also provides a composition comprising at least three distinct antibodies, or antigen-binding fragments thereof, wherein the three distinct antibodies or antigen-binding fragments bind to distinct, non-overlapping epitopes of Ara h 2, wherein the composition comprises at least three of the following:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
(iii) an antibody, or an antigen-binding fragment thereof, comprising a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
(iv) an antibody, or an antigen-binding fragment thereof, comprising a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30.

Preferably, the composition comprises at least three of the following:
(i) an antibody, or an antigen-binding fragment thereof, comprising a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
(iii) an antibody, or an antigen-binding fragment thereof, comprising a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
(iv) an antibody, or an antigen-binding fragment thereof, comprising a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30.

More preferably, the composition comprises at least three of the following:
(i) an antibody, or an antigen-binding fragment thereof, comprising a VH having at least 70% identity to any one of SEQ ID NOs 7, 44 and 45; and a VL having at least 70% identity to any one of SEQ ID NOs 8, 46, 47, 48 and 49;
(ii) an antibody, or an antigen-binding fragment thereof, comprising a VH having at least 70% identity to any one of SEQ ID NOs 15, 37 and 38; and a VL having at least 70% identity to any one of SEQ ID NOs 16, 39 and 40;
(iii) an antibody, or an antigen-binding fragment thereof, comprising a VH having at least 70% identity to any one of SEQ ID NOs 23, 41, 50 and 53; and a VL having at least 70% identity to any one of SEQ ID NOs 24, 42 and 43;
(iv) an antibody, or an antigen-binding fragment thereof, comprising a VH having at least 70% identity to any one of SEQ ID NOs 31, 33 and 34; and a VL having at least 70% identity to any one of SEQ ID NOs 32, 35, 36 and 54.

Even more preferably, the composition comprises at least three of the following:
(i) an antibody, or an antigen-binding fragment thereof, comprising a VH according to any one of SEQ ID NOs 7, 44 and 45; and a VL according to any one of SEQ ID NOs 8, 46, 47, 48 and 49;
(ii) an antibody, or an antigen-binding fragment thereof, comprising a VH according to any one of SEQ ID NOs 15, 37 and 38; and a VL according to any one of SEQ ID NOs 16, 39 and 40;
(iii) an antibody, or an antigen-binding fragment thereof, comprising a VH according to any one of SEQ ID NOs 23, 41, 50 and 53; and a VL according to any one of SEQ ID NOS 24, 42 and 43;
(iv) an antibody, or an antigen-binding fragment thereof, comprising a VH according to any one of SEQ ID NOs 31, 33 and 34; and a VL according to any one of SEQ ID NOs 32, 35, 36 and 54.

In some embodiments, the composition comprises:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40; and
(iii) an antibody, or an antigen-binding fragment thereof, comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43.

In some embodiments, the composition comprises:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(iii) an antibody, or an antigen-binding fragment thereof, comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antibody, or an antigen-binding fragment thereof, comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

In some embodiments, the composition comprises:
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40;
(iii) an antibody, or an antigen-binding fragment thereof, comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antibody, or an antigen-binding fragment thereof, comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

Preferably, the composition comprises:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40; and
(iv) an antibody, or an antigen-binding fragment thereof, comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

Preferably, the composition comprises antibodies binding to four distinct, non-overlapping epitopes on Ara h 2. Accordingly, the composition may comprise the four distinct antibodies, or antigen-binding fragments, according to (i), (ii), (iii) and (iv).

Preferably, the composition comprises:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40;
(iii) an antibody, or an antigen-binding fragment thereof, comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOs 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antibody, or an antigen-binding fragment thereof, comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

More preferably, the composition comprises:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6; in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14; in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
(iii) an antibody, or an antigen-binding fragment thereof, comprising a CDRH1 having at least 70% identity to according to SEQ ID NO: 17, a CDRH2 having at least 70% identity to SEQ ID NO: 18, a CDRH3 having at least 70% identity to SEQ ID NO: 51, a CDRL1 having at least 70% identity to SEQ ID NO: 20, a CDRL2 having at least 70% identity to SEQ ID NO: 21, and a CDRL3 having at least 70% identity to SEQ ID NO: 22; in particular a CDRH1 according to according to SEQ ID NO: 17, a CDRH2 according to SEQ ID NO: 18, a CDRH3 according to SEQ ID NO: 51, a CDRL1 according to SEQ ID NO: 20, a CDRL2 according to SEQ ID NO: 21, and a CDRL3 according to SEQ ID NO: 22; and
(iv) an antibody, or an antigen-binding fragment thereof, comprising a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30; in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30.

Even more preferably, the composition comprises:
(i) an antibody, or an antigen-binding fragment thereof, comprising a VH having at least 70% identity to SEQ ID NO: 45; and a VL having at least 70% identity to SEQ ID NO: 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained; such as a VH according to SEQ ID NO: 45, and a VL according to SEQ ID NO: 49;
(ii) an antibody, or an antigen-binding fragment thereof, comprising a VH having at least 70% identity to SEQ ID NO: 38; and a VL having at least 70% identity to SEQ ID NO: 39; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained; such as a VH according to SEQ ID NO: 38, and a VL according to SEQ ID NO: 39;
(iii) an antibody, or an antigen-binding fragment thereof, comprising a VH having at least 70% identity to SEQ ID NO: 53; and a VL having at least 70% identity to SEQ ID NO: 43; wherein the CDRH1 according to according to SEQ ID NO: 17, CDRH2 according to SEQ ID NO: 18, CDRH3 according to SEQ ID NO: 51, CDRL1 according to SEQ ID NO: 20, CDRL2 according to SEQ ID NO: 21, and CDRL3 according to SEQ ID NO: 22 are preferably maintained; such as a VH according to SEQ ID NO: 53, and a VL according to SEQ ID NO: 43; and
(iv) an antibody, or an antigen-binding fragment thereof, comprising a VH having at least 70% identity to SEQ ID NO: 34; and a VL having at least 70% identity to SEQ ID NO: 35 or 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained; such as a VH according to SEQ ID NO: 34, and a VL according to SEQ ID NO: 35 or 54.

As described above, the composition of the invention preferably comprises antibodies binding to at least three, preferably four, distinct non-overlapping epitopes of Ara h 2. The antibodies, or the antigen-binding fragments thereof, which are comprised in the composition, may be selected from the four different antibodies 17H9, 15E3, 2F8 and 7G6, or the variants thereof, as described above, in particular as shown in Tables 1 and 2. Preferably, the composition therefore comprises three or four (monospecific) antibodies as described herein, in particular selected from the four different antibodies 17H9, 15E3, 2F8 and 7G6, or the variants thereof, as described above, in particular as shown in Tables 1 and 2. In some embodiments, the composition comprises multispecific antibodies, in particular as described herein.

Binding to three different epitopes of Ara h 2 may be obtained in compositions including three distinct antigen-binding sites, in particular by a composition comprising:
(i) three distinct monospecific antibodies, or antigen-binding fragments thereof, binding to three distinct epitopes of Ara h 2, in particular selected from 17H9, 15E3, 2F8 and 7G6, or the variants thereof, as described above, in particular as shown in Tables 1 and 2;
(ii) (1) a bispecific antibody, or a bispecific antigen-binding fragment, in particular having two specificities selected from 17H9, 15E3, 2F8 and 7G6, or the variants thereof, as described above, in particular as shown in Tables 1 and 2, and (2) an additional monospecific antibody, in particular selected from 17H9, 15E3, 2F8 and 7G6, or the variants thereof, as described above, in particular as shown in Tables 1 and 2, binding to another epitope on Ara h 2; or
(iii) a trispecific antibody, or a trispecific antigen-binding fragment, in particular having three specificities selected from 17H9, 15E3, 2F8 and 7G6, or the variants thereof, as described above, in particular as shown in Tables 1 and 2.

Binding to four different epitopes of Ara h 2 may be obtained in compositions including four distinct antigen-binding sites, in particular by a composition comprising:
(i) four distinct monospecific antibodies, or antigen-binding fragments thereof, binding to three distinct epitopes of Ara h 2, in particular selected from 17H9, 15E3, 2F8 and 7G6, or the variants thereof, as described above, in particular as shown in Tables 1 and 2;
(ii) (1) a bispecific antibody, or a bispecific antigen-binding fragment, in particular having two specificities selected from 17H9, 15E3, 2F8 and 7G6, or the variants thereof, as described above, in particular as shown in Tables 1 and 2, and (2) two additional monospecific antibodies, in particular selected from 17H9, 15E3, 2F8 and 7G6, or the variants thereof, as described above, in particular as shown in Tables 1 and 2, binding to further distinct epitopes on Ara h 2;
(iii) two distinct bispecific antibodies, or bispecific antigen-binding fragments, in particular wherein each of the bispecific antibodies, or bispecific antigen-binding fragments, has two distinct specificities selected from 17H9, 15E3, 2F8 and 7G6, or the variants thereof, as described above, in particular as shown in Tables 1 and 2
(iv) (1) a trispecific antibody, or a trispecific antigen-binding fragment, in particular having three specificities selected from 17H9, 15E3, 2F8 and 7G6, or the variants thereof, as described above, in particular as shown in Tables 1 and 2, and (2) an additional monospecific antibody, in particular selected from 17H9, 15E3, 2F8 and 7G6, or the variants thereof, as described above, in particular as shown in Tables 1 and 2, binding to another epitope on Ara h 2; or
(v) a tetraspecific antibody, or a tetraspecific antigen-binding fragment, in particular having the four specificities of 17H9, 15E3, 2F8 and 7G6, or the variants thereof, as described above, in particular as shown in Tables 1 and 2.

Alternatively or additionally, the composition may further comprise at least one additional agent useful for treating peanut allergy. The additional agent useful for treating peanut allergy may be selected from the group comprising: a β-adrenergic agonist (e.g. epinephrine), antihistamine, a corticosteroid, an anti-IgE antibody, an anti-IgE antibody binding fragment, a peptide vaccine and further antibodies capable of binding to a peanut allergen. In some embodiments, the composition comprises a β-adrenergic agonist, such as epinephrine.

In some embodiments, the composition comprises a peanut allergen. The peanut allergen may be untreated or treated peanut, such as peanut powder, roasted peanut or peanut butter. In some embodiments, the peanut allergen may be a commercially available product, such as Palforzia (defatted powder of peanuts). The peanut allergen may be a peanut protein, such as Ara h 1, Ara h 2, Ara h 3, Ara h 4, Ara h 5, Ara h 6/7, Ara h 8, Ara h 9 and Ara h 10/11. In a specific embodiment the peanut protein may be in particular selected from Ara h 2, Ara h 3 and Ara h 6, or a combination thereof. The peanut allergen may be of peanut origin, recombinantly expressed or is a synthetic peanut peptide. In some embodiments, the anti-peanut allergen antibodies, or antigen-binding fragments thereof, as described herein, may be pre-incubated with the peanut allergen and may be administered as a mixture to a subject.

Combining a peanut allergen, preferably selected from the group consisting of Ara h 1, Ara h 2, Ara h 3, Ara h 4, Ara h 5, Ara h 6/7, Ara h 8, Ara h 9 and Ara h 10/11 or a mixture thereof, or more preferably selected from the group consisting of Ara h1, Ara h2, Ara h3 and Ara h6 or a mixture thereof, with the antibodies of the invention increases the safety of administering the allergen (e.g. in desensitization). Furthermore, without being bound to any theory, the present inventors assume that the combination of antibodies with the respective allergen (targeted by the antibodies), i.e. the combination of passive (antibodies) and active (allergen) immunization, has a synergistic effect. The components, i.e. the allergen component and the antibody component, may be administered simultaneously or in a timely staggered manner, e.g. by administering the components separately from one another, e.g. within 15, 30, 60 or 90 min.

The present invention also provides a diagnostic composition comprising an antibody according to the present invention, a nucleic acid(s) according to the present invention, a vector(s) according to the present invention, and/or a cell according to the present invention. The diagnostic composition may optionally comprise suitable means for detection, such as reagents conventionally used in immuno- or nucleic acid based diagnostic methods.

The antibodies described herein are, for example, suited for diagnostic purposes. Accordingly, they may be used in immunoassays, in which they can be utilized in liquid phase or bound to a solid phase carrier. Such immunoassays may be competitive or non-competitive immunoassays; in either a direct or in an indirect format. Examples of such immunoassays include, but are not limited to, radioimmunoassay (RIA), enzyme-linked immunoassay (ELISA), sandwich (immunometric assay), immunohistochemistry, flow cytometry and Western blot assay. To this end, the antibody may be labelled, e.g. as described above.

In a further aspect the present invention also provides a kit comprising one or more of
(i) the antibody according to the present invention, or an antigen-binding fragment thereof, as described above,
(ii) the nucleic acid molecule (or the plurality of nucleic acid molecules) according to the present invention as described above,
(iii) the vector (or the plurality of vectors) according to the present invention as described above,
(iv) the cell according to the present invention as described above, and/or
(v) the composition according to the present invention as described above.

In addition, the kit may comprise means for administration of the antibody, or an antigen binding fragment thereof, according to the present invention, the nucleic acid according to the present invention, the vector according to the present invention, the cell according to the present invention or the pharmaceutical composition according to the present invention, such as a syringe or a vessel, a leaflet, and/or a co-agent to be administered as described herein. For example, the kit may contain a leaflet, e.g. comprising instructions for use. In addition or alternatively, the kit may comprise one or more reagents, e.g. for use in appropriate diagnostic assays. In some instances, the kit may contain a reference agent or control. In some embodiments, the composition of the invention may be provided in kit form, e.g., designed such that a combined composition is reconstituted just prior to administration to a subject. For example, a lyophilized antibody may be provided in kit form with sterile water or a sterile buffer (e.g., in a separate container).

In some embodiments, the kit comprises at least two distinct antibodies (or nucleic acids encoding such antibodies or compositions comprising such antibodies), wherein the distinct antibodies may be provided in distinct containers. Preferably, the kit comprises at least two distinct antibodies, or antigen-binding fragments thereof, binding to distinct, non-overlapping epitopes of Ara h 2 (or nucleic acids encoding such antibodies or compositions comprising such antibodies). More preferably, the kit comprises (exactly) three distinct antibodies, or antigen-binding fragments thereof, binding to distinct, non-overlapping epitopes of Ara h 2 (or nucleic acids encoding such antibodies or compositions comprising such antibodies). Even more preferably, the kit comprises (exactly) four distinct antibodies, or antigen-binding fragments thereof, binding to distinct, non-overlapping epitopes of Ara h 2 (or nucleic acids encoding such antibodies or compositions comprising such antibodies). To this end, the antibodies contained in the kit may be selected from the four different antibodies 17H9, 15E3, 2F8 and 7G6, or the variants thereof, as described above, e.g. in Tables 1 and 2. As shown in the appended examples, 17H9, 15E3, 2F8 and 7G6 bind to distinct, non-overlapping epitopes of Ara h 2.

Accordingly, the kit may comprise
(i) at least two distinct antibodies, or antigen-binding fragments thereof, according to any the present invention as described above, which bind to distinct, non-overlapping epitopes of Ara h 2;
(ii) nucleic acid molecule(s) according to any the present invention as described above, which encode at least two distinct antibodies, or antigen-binding fragments thereof, which bind to distinct, non-overlapping epitopes of Ara h 2; or
(iii) composition(s) comprising (i) or (ii).

While the following description refers to kits comprising distinct antibodies, or antigen-binding fragments thereof, it is understood that the kits may likewise comprise (distinct) nucleic acid(s) encoding said antibodies, or antigen-binding fragments; or (distinct) composition(s) comprising said antibodies, or antigen-binding fragments, or said nucleic acid(s).

In particular, the kit may comprise at least two of the following:
(i) the antibody, or an antigen-binding fragment thereof, comprising (a) the CDRH1, sequences of 17H9, or sequence variants thereof, as described above;
(ii) the antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above;
(iii) the antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and/or
(iv) the antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the kit comprises (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above.

In some embodiments, the kit comprises (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above.

In some embodiments, the kit comprises (exactly) two distinct antibodies, namely:
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the kit comprises (exactly) two distinct antibodies, namely:
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

Preferably, the kit comprises (exactly) two distinct antibodies, namely:
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above.

More preferably, the kit comprises (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

Preferably, the kit comprises (exactly) three or four distinct antibodies, or antigen-binding fragments thereof, according to any one of claims 1 to 42, which bind to distinct, non-overlapping epitopes of Ara h 2; or nucleic acid(s) encoding said antibodies or compositions comprising said antibodies.

In some embodiments, the kit comprises (exactly) three distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above.

In some embodiments, the kit comprises (exactly) three distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above;
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the kit comprises (exactly) three distinct antibodies, namely:
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above;
(iii) an antibody, or an antigen-binding fragment thereof, comprising (a) the CDRH1, sequences of 2F8, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

Preferably, the kit comprises (exactly) three distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

It is also preferred that the kit comprises (exactly) four distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above;
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the kit may comprise (i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and (ii) an antibody, or an antigen-binding fragment thereof, comprising (a) the CDRH1, CDRH2, CDRH3, CDRL1, CDRL2 and CDRL3 sequences and/or (b) the VH and VL sequences of (wild-type) 7G6, or sequence variants thereof, as described herein, e.g. in Tables 1 and 2. Optionally, such a kit may further comprise an antibody, or an antigen-binding fragment thereof, including the CDR and/or VH/VL sequences of 15E3 (or a sequence variant thereof), and/or an antibody, or an antigen-binding fragment thereof, including the CDR and/or VH/VL sequences of 2F8 (or a sequence variant thereof).

In some embodiments, the kit may comprise (i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and (ii) an antibody, or an antigen-binding fragment thereof, comprising (a) the CDRH1, CDRH2, CDRH3, CDRL1, CDRL2 and CDRL3 sequences and/or (b) the VH and VL sequences of (wild-type) 2F8, or sequence variants thereof, as described herein, e.g. in Tables 1 and 2. Optionally, such a kit may further comprise an antibody, or an antigen-binding fragment thereof, including the CDR and/or VH/VL sequences of 15E3 (or a sequence variant thereof), and/or an antibody, or an antigen-binding fragment thereof, including the CDR and/or VH/VL sequences of 7G6 (or a sequence variant thereof).

In some embodiments, the kit may comprise (i) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and (ii) an antibody, or an antigen-binding fragment thereof, comprising (a) the CDRH1, CDRH2, CDRH3, CDRL1, CDRL2 and CDRL3 sequences and/or (b) the VH and VL sequences of (wild-type) 7G6, or sequence variants thereof, as described herein, e.g. in Tables 1 and 2. Optionally, such a kit may further comprise an antibody, or an antigen-binding fragment thereof, including the CDR and/or VH/VL sequences of 17H9 (or a sequence variant thereof), and/or an antibody, or an antigen-binding fragment thereof, including the CDR and/or VH/VL sequences of 2F8 (or a sequence variant thereof).

In some embodiments, the kit may comprise (i) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and (ii) an antibody, or an antigen-binding fragment thereof, comprising (a) the CDRH1, CDRH2, CDRH3, CDRL1, CDRL2 and CDRL3 sequences and/or (b) the VH and VL sequences of (wild-type) 2F8, or sequence variants thereof, as described herein, e.g. in Tables 1 and 2. Optionally, such a kit may further comprise an antibody, or an antigen-binding fragment thereof, including the CDR and/or VH/VL sequences of 17H9 (or a sequence variant thereof), and/or an antibody, or an antigen-binding fragment thereof, including the CDR and/or VH/VL sequences of 7G6 (or a sequence variant thereof).

The present invention also provides a kit comprising at least three distinct antibodies, or antigen-binding fragments thereof, wherein the three distinct antibodies or antigen-binding fragments bind to distinct, non-overlapping epitopes of Ara h 2, wherein the kit comprises at least three of the following:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
(iii) an antibody, or an antigen-binding fragment thereof, comprising a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
(iv) an antibody, or an antigen-binding fragment thereof, comprising a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30.

Preferably, the kit comprises at least three of the following:
(i) an antibody, or an antigen-binding fragment thereof, comprising a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
(iii) an antibody, or an antigen-binding fragment thereof, comprising a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
(iv) an antibody, or an antigen-binding fragment thereof, comprising a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30.

More preferably, the kit comprises at least three of the following:
(i) an antibody, or an antigen-binding fragment thereof, comprising a VH having at least 70% identity to any one of SEQ ID NOs 7, 44 and 45; and a VL having at least 70% identity to any one of SEQ ID NOs 8, 46, 47, 48 and 49;
(ii) an antibody, or an antigen-binding fragment thereof, comprising a VH having at least 70% identity to any one of SEQ ID NOs 15, 37 and 38; and a VL having at least 70% identity to any one of SEQ ID NOs 16, 39 and 40;
(iii) an antibody, or an antigen-binding fragment thereof, comprising a VH having at least 70% identity to any one of SEQ ID NOs 23, 41, 50 and 53; and a VL having at least 70% identity to any one of SEQ ID NOs 24, 42 and 43;
(iv) an antibody, or an antigen-binding fragment thereof, comprising a VH having at least 70% identity to any one of SEQ ID NOs 31, 33 and 34; and a VL having at least 70% identity to any one of SEQ ID NOs 32, 35, 36 and 54.

Even more preferably, the kit comprises at least three of the following:
(i) an antibody, or an antigen-binding fragment thereof, comprising a VH according to any one of SEQ ID NOs 7, 44 and 45; and a VL according to any one of SEQ ID NOs 8, 46, 47, 48 and 49;
(ii) an antibody, or an antigen-binding fragment thereof, comprising a VH according to any one of SEQ ID NOs 15, 37 and 38; and a VL according to any one of SEQ ID NOs 16, 39 and 40;
(iii) an antibody, or an antigen-binding fragment thereof, comprising a VH according to any one of SEQ ID NOs 23, 41, 50 and 53; and a VL according to any one of SEQ ID NOS 24, 42 and 43;
(iv) an antibody, or an antigen-binding fragment thereof, comprising a VH according to any one of SEQ ID NOs 31, 33 and 34; and a VL according to any one of SEQ ID NOs 32, 35, 36 and 54.

In some embodiments, the kit comprises:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40; and
(iii) an antibody, or an antigen-binding fragment thereof, comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43.

In some embodiments, the kit comprises:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(iii) an antibody, or an antigen-binding fragment thereof, comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antibody, or an antigen-binding fragment thereof, comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

In some embodiments, the kit comprises:
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40;
(iii) an antibody, or an antigen-binding fragment thereof, comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antibody, or an antigen-binding fragment thereof, comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

Preferably, the kit comprises:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40; and
(iv) an antibody, or an antigen-binding fragment thereof, comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

Preferably, the kit comprises antibodies binding to four distinct, non-overlapping epitopes on Ara h 2. Accordingly, the kit may comprise the four distinct antibodies, or antigen-binding fragments, according to (i), (ii), (iii) and (iv).

Preferably, the kit comprises:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40;
(iii) an antibody, or an antigen-binding fragment thereof, comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOs 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antibody, or an antigen-binding fragment thereof, comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

More preferably, the kit comprises:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6; in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14; in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
(iii) an antibody, or an antigen-binding fragment thereof, comprising a CDRH1 having at least 70% identity to according to SEQ ID NO: 17, a CDRH2 having at least 70% identity to SEQ ID NO: 18, a CDRH3 having at least 70% identity to SEQ ID NO: 51, a CDRL1 having at least 70% identity to SEQ ID NO: 20, a CDRL2 having at least 70% identity to SEQ ID NO: 21, and a CDRL3 having at least 70% identity to SEQ ID NO: 22; in particular a CDRH1 according to according to SEQ ID NO: 17, a CDRH2 according to SEQ ID NO: 18, a CDRH3 according to SEQ ID NO: 51, a CDRL1 according to SEQ ID NO: 20, a CDRL2 according to SEQ ID NO: 21, and a CDRL3 according to SEQ ID NO: 22; and
(iv) an antibody, or an antigen-binding fragment thereof, comprising a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30; in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30.

Even more preferably, the kit comprises:
(i) an antibody, or an antigen-binding fragment thereof, comprising a VH having at least 70% identity to SEQ ID NO: 45; and a VL having at least 70% identity to SEQ ID NO: 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained; such as a VH according to SEQ ID NO: 45, and a VL according to SEQ ID NO: 49;
(ii) an antibody, or an antigen-binding fragment thereof, comprising a VH having at least 70% identity to SEQ ID NO: 38; and a VL having at least 70% identity to SEQ ID NO: 39; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained; such as a VH according to SEQ ID NO: 38, and a VL according to SEQ ID NO: 39;
(iii) an antibody, or an antigen-binding fragment thereof, comprising a VH having at least 70% identity to SEQ ID NO: 53; and a VL having at least 70% identity to SEQ ID NO: 43; wherein the CDRH1 according to according to SEQ ID NO: 17, CDRH2 according to SEQ ID NO: 18, CDRH3 according to SEQ ID NO: 51, CDRL1 according to SEQ ID NO: 20, CDRL2 according to SEQ ID NO: 21, and CDRL3 according to SEQ ID NO: 22 are preferably maintained; such as a VH according to SEQ ID NO: 53, and a VL according to SEQ ID NO: 43; and
(iv) an antibody, or an antigen-binding fragment thereof, comprising a VH having at least 70% identity to SEQ ID NO: 34; and a VL having at least 70% identity to SEQ ID NO: 35 or 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained; such as a VH according to SEQ ID NO: 34, and a VL according to SEQ ID NO: 35 or 54.

As described above, the kit of the invention preferably comprises antibodies binding to at least three, preferably four, distinct non-overlapping epitopes of Ara h 2. The antibodies, or the antigen-binding fragments thereof, which are comprised in the kit, may be selected from the four different antibodies 17H9, 15E3, 2F8 and 7G6, or the variants thereof, as described above, in particular as shown in Tables 1 and 2. Preferably, the kit therefore comprises three or four (monospecific) antibodies as described herein, in particular selected from the four different antibodies 17H9, 15E3, 2F8 and 7G6, or the variants thereof, as described above, in particular as shown in Tables 1 and 2. In some embodiments, the kit comprises multispecific antibodies, in particular as described herein.

Binding to three different epitopes of Ara h 2 may be obtained in kits including three distinct antigen-binding sites, in particular by a kit comprising:
(i) three distinct monospecific antibodies, or antigen-binding fragments thereof, binding to three distinct epitopes of Ara h 2, in particular selected from 17H9, 15E3, 2F8 and 7G6, or the variants thereof, as described above, in particular as shown in Tables 1 and 2;
(ii) (1) a bispecific antibody, or a bispecific antigen-binding fragment, in particular having two specificities selected from 17H9, 15E3, 2F8 and 7G6, or the variants thereof, as described above, in particular as shown in Tables 1 and 2, and (2) an additional monospecific antibody, in particular selected from 17H9, 15E3, 2F8 and 7G6, or the variants thereof, as described above, in particular as shown in Tables 1 and 2, binding to another epitope on Ara h 2; or
(iii) a trispecific antibody, or a trispecific antigen-binding fragment, in particular having three specificities selected from 17H9, 15E3, 2F8 and 7G6, or the variants thereof, as described above, in particular as shown in Tables 1 and 2.

Binding to four different epitopes of Ara h 2 may be obtained in kits including four distinct antigen-binding sites, in particular by a kit comprising:
(i) four distinct monospecific antibodies, or antigen-binding fragments thereof, binding to three distinct epitopes of Ara h 2, in particular selected from 17H9, 15E3, 2F8 and 7G6, or the variants thereof, as described above, in particular as shown in Tables 1 and 2;
(ii) (1) a bispecific antibody, or a bispecific antigen-binding fragment, in particular having two specificities selected from 17H9, 15E3, 2F8 and 7G6, or the variants thereof, as described above, in particular as shown in Tables 1 and 2, and (2) two additional monospecific antibodies, in particular selected from 17H9, 15E3, 2F8 and 7G6, or the variants thereof, as described above, in particular as shown in Tables 1 and 2, binding to further distinct epitopes on Ara h 2;
(iii) two distinct bispecific antibodies, or bispecific antigen-binding fragments, in particular wherein each of the bispecific antibodies, or bispecific antigen-binding fragments, has two distinct specificities selected from 17H9, 15E3, 2F8 and 7G6, or the variants thereof, as described above, in particular as shown in Tables 1 and 2;
(iv) (1) a trispecific antibody, or a trispecific antigen-binding fragment, in particular having three specificities selected from 17H9, 15E3, 2F8 and 7G6, or the variants thereof, as described above, in particular as shown in Tables 1 and 2, and (2) an additional monospecific antibody, in particular selected from 17H9, 15E3, 2F8 and 7G6, or the variants thereof, as described above, in particular as shown in Tables 1 and 2, binding to another epitope on Ara h 2; or
(v) a tetraspecific antibody, or a tetraspecific antigen-binding fragment, in particular having the four specificities of 17H9, 15E3, 2F8 and 7G6, or the variants thereof, as described above, in particular as shown in Tables 1 and 2.

Alternatively or additionally, the kit may further comprise at least one additional agent useful for treating peanut allergy, which may be provided in a separate container. The additional agent useful for treating peanut allergy may be selected from the group comprising: a β-adrenergic agonist (e.g. epinephrine), antihistamine, a corticosteroid, an anti-IgE antibody, an anti-IgE antibody binding fragment, a peptide vaccine and further antibodies capable of binding to a peanut allergen. In some embodiments, the kit comprises a β-adrenergic agonist, such as epinephrine.

In some embodiments, the kit comprises a peanut allergen. The peanut allergen may be untreated or treated peanut, such as peanut powder, roasted peanut or peanut butter. In some embodiments, the peanut allergen may be a commercially available product, such as Palforzia (defatted powder of peanuts). The peanut allergen may be a peanut protein, such as Ara h 1, Ara h 2, Ara h 3, Ara h 4, Ara h 5, Ara h 6/7, Ara h 8, Ara h 9 and Ara h 10/11. In a specific embodiment the peanut protein may be in particular selected from Ara h 2, Ara h 3 and Ara h 6, or a combination thereof. The peanut allergen may be of peanut origin, recombinantly expressed or is a synthetic peanut peptide. In some embodiments, the anti-peanut allergen antibodies, or antigen-binding fragments thereof, as described herein, may be provided (in a separate container) with instructions for pre-incubation with the peanut allergen before it is administered as a mixture to a subject. In other embodiments, the anti-peanut allergen antibodies, or antigen-binding fragments thereof, as described herein, may be provided (in a separate container) with instructions for separate administration with the peanut allergen in a combined treatment schedule.

### Medical Treatments and Other Uses

In a further aspect, the present invention provides the use of the antibody according to the present invention, or an antigen-binding fragment thereof, the nucleic acid molecule (or the plurality of nucleic acid molecules) according to the present invention, the vector (or the plurality of vectors) according to the present invention, the cell according to the present invention or the (pharmaceutical) composition according to the present invention as a medicament. In particular, the antibody according to the present invention, or an antigen-binding fragment thereof, the nucleic acid molecule (or the plurality of nucleic acid molecules) according to the present invention, the vector (or the plurality of vectors) according to the present invention, the cell according to the present invention or the (pharmaceutical) composition according to the present invention may be used in prophylaxis and/or treatment of a peanut allergy or of a symptom of a peanut allergy, such as an anaphylactic reaction due to a peanut allergy.

Accordingly, the present invention also provides a method of treating, ameliorating or reducing a peanut allergy or a symptom of a peanut allergy, such as an anaphylactic reaction due to a peanut allergy, or lowering the risk of (occurrence of) a peanut allergy or a symptom of a peanut allergy, such as an anaphylactic reaction due to a peanut allergy, comprising: administering to a subject (in need thereof), (a therapeutically effective amount of) an antibody, or an antigen-binding fragment thereof, according to the present invention, a nucleic acid molecule (or the plurality of nucleic acid molecules) according to the present invention, a vector (or the plurality of vectors) according to the present invention, a cell according to the present invention or a (pharmaceutical) composition according to the present invention. Moreover, the present invention also provides the use of an antibody according to the present invention, or an antigen-binding fragment thereof, a nucleic acid molecule (or the plurality of nucleic acid molecules) according to the present invention, a vector (or the plurality of vectors) according to the present invention, a cell according to the present invention, or a pharmaceutical composition according to the present invention in the manufacture of a medicament for prophylaxis, treatment or attenuation of a peanut allergy or of a symptom of a peanut allergy, such as an anaphylactic reaction due to a peanut allergy.

As used herein, the terms “treat” or “treatment” include therapeutic treatment and prophylactic or preventative measures. Prophylaxis of a peanut allergy refers in particular to prophylactic settings, wherein the subject was either not diagnosed with a peanut allergy (either no diagnosis was performed or diagnosis results were negative) and/or the subject does not show symptoms of a peanut allergy. Prophylaxis of a symptom of a peanut allergy, such as an anaphylactic reaction due to a peanut allergy, refers in particular to prophylactic settings, wherein the subject is not currently experiencing a symptom of a peanut allergy, such as an anaphylactic reaction due to a peanut allergy, but may possibly expect to experience a symptom of a peanut allergy, such as an anaphylactic reaction due to a peanut allergy, in the (near) future, e.g. due to the contact with substances (e.g., food intake) of unknown components or known to contain peanut (components). In therapeutic settings, in contrast, the subject is typically diagnosed with a peanut allergy and/or showing symptoms of a peanut allergy. Of note, the terms “treatment” and “therapy”/“therapeutic” include (complete) cure as well as attenuation/reduction of a peanut allergy and/or related symptoms.

In general, the object of the “treatment” may be to decrease, ameliorate, inhibit, prevent or slow down (lessen or delay) an undesired physiological change or disorder, such as the peanut allergy or a symptom of a peanut allergy, such as an anaphylactic reaction due to a peanut allergy. Beneficial or desired clinical results of a treatment include, but are not limited to, alleviation of symptoms, diminishment of the extent of a disease, stabilized (i.e., not worsening) state of disease, delay or slowing of disease progression, amelioration or palliation of the disease state, and remission (whether partial or total), whether detectable or undetectable. “Treatment” can also mean prolonging survival, e.g. as compared to expected survival if not receiving treatment. Those in need of treatment include those already with the condition or disorder as well as those prone to have the condition or disorder or those in which the manifestation of the condition or disorder or the risk thereof is to be decreased, delayed or prevented.

In some embodiments the subject may be a human. The human may be selected from the group of a human suffering from peanut allergy, a peanut-sensitized human without clinical relevant allergy, a human suffering from peanut allergy that underwent immunotherapy, a human at risk of developing a peanut allergy and a human of unknown clinical history for peanut allergy.

Symptoms of peanut allergy may include one or more of skin rash, itching skin, itching or tingling sensation in or around the mouth or throat, headache, sneezing, swelling, nausea, diarrhea or anaphylaxis. Whether or not a subject suffers from peanut allergy may be determined by correlation of allergic symptoms to contact with (e.g. intake of) peanuts or products containing components of peanuts. Accordingly, diagnosis may include a food diary and/or an elimination diet. Additionally or alternatively, an allergen skin test and/or a blood test (for peanut-allergy IgE) may be performed.

One way of checking efficacy of therapeutic treatment involves monitoring disease symptoms after administration of the antibody or of the composition. Treatment can be a single dose schedule or a multiple dose schedule. In some embodiments, an antibody, antibody fragment, nucleic acid, vector, cell, or composition as described herein may be administered to a subject in need of such treatment. Such a subject includes, but is not limited to, one who is particularly at risk of, or susceptible to, a peanut allergy or a symptom of a peanut allergy, such as an anaphylactic reaction due to a peanut allergy.

The antibody according to the present invention, or an antigen-binding fragment thereof, the nucleic acid molecule (or the plurality of nucleic acid molecules) according to the present invention, the vector (or the plurality of vectors) according to the present invention, the cell according to the present invention, or the pharmaceutical composition according to the present invention may be administered by any route of administration including, but not limited to, oral, intravenous, intramuscular, intra-arterial, intraperitoneal, transdermal, transcutaneous, topical, subcutaneous, intranasal, enteral, sublingual or rectal routes. In addition, any gene therapy approaches may be used, e.g. the antibody according to the present invention, or an antigen-binding fragment thereof, may be administered as nucleic acid or vector encoding said antibody, e.g. using viral or non-viral vectors as described above. In some embodiments, the antibody according to the present invention, or an antigen-binding fragment thereof, the nucleic acid molecule (or the plurality of nucleic acid molecules) according to the present invention, the vector (or the plurality of vectors) according to the present invention, the cell according to the present invention or the pharmaceutical composition according to the present invention may be administered systemically, for example by intravenous or subcutaneous administration.

### Combination Treatments

In some embodiments, co-administration or sequential administration of (i) an antibody according to the present invention, or an antigen-binding fragment thereof, a nucleic acid molecule (or the plurality of nucleic acid molecules) according to the present invention, a vector (or the plurality of vectors) according to the present invention, a cell according to the present invention or the pharmaceutical composition according to the present invention and (ii) a co-agent may be desirable. In some instances, the co-agent may be comprised in the pharmaceutical composition.

Preferably, the above-described medical uses comprise (combined) administration of at least two, three or four distinct antibodies or antigen-binding fragments thereof, which bind to distinct non-overlapping epitopes of Ara h 2; or nucleic acid(s) or vector(s) encoding the distinct antibodies; or composition(s) comprising the distinct antibodies.

While the following description refers to administration of distinct antibodies, or antigen-binding fragments thereof, it is understood that the administration may likewise refer to (distinct) nucleic acid(s) encoding said antibodies, or antigen-binding fragments; or (distinct) composition(s) comprising said antibodies, or antigen-binding fragments, or said nucleic acid(s).

For combination, the distinct antibodies may be administered in the same or in different compositions. The distinct antibodies may be administered via the same or via distinct routes of administration. As used herein, “combined administration” means that the distinct antibodies are administered in a combined treatment scheme, i.e. a treatment scheme including the administration of the distinct antibodies (in contrast to a treatment scheme relating to a single antibody only, followed by a treatment scheme with another (single) antibody). Thereby, the antibodies may be administered simultaneously (e.g., during the same treatment session) or sequentially, e.g. on different days.

Preferably, the above-described medical uses or treatments comprise (combined) administration of at least two distinct antibodies, or antigen-binding fragments thereof, binding to distinct, non-overlapping epitopes of Ara h 2. More preferably, the above-described medical uses comprise (combined) administration of (exactly) three distinct antibodies, or antigen-binding fragments thereof, binding to distinct, non-overlapping epitopes of Ara h 2. Even more preferably, the above-described medical uses comprise (combined) administration of (exactly) four distinct antibodies, or antigen-binding fragments thereof, binding to distinct, non-overlapping epitopes of Ara h 2. To this end, the antibodies administered in combination may be selected from the four different antibodies 17H9, 15E3, 2F8 and 7G6, or the variants thereof, as described above, e.g. in Tables 1 and 2. As shown in the appended examples, 17H9, 15E3, 2F8 and 7G6 bind to distinct, non-overlapping epitopes of Ara h 2.

Accordingly, the medical use or treatment may comprise (combined) administration of at least two of the following:
(i) the antibody, or an antigen-binding fragment thereof, comprising (a) the CDRH1, sequences of 17H9, or sequence variants thereof, as described above;
(ii) the antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above;
(iii) the antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and/or
(iv) the antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the medical use or treatment may comprise (combined) administration of (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above.

In some embodiments, the medical use or treatment may comprise (combined) administration of (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above.

In some embodiments, the medical use or treatment may comprise (combined) administration of (exactly) two distinct antibodies, namely:
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the medical use or treatment may comprise (combined) administration of (exactly) two distinct antibodies, namely:
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

Preferably, the medical use or treatment may comprise (combined) administration of (exactly) two distinct antibodies, namely:
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above.

More preferably, the medical use or treatment may comprise (combined) administration of (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

Preferably, the medical use or treatment may comprise (combined) administration of (exactly) three or four distinct antibodies, or antigen-binding fragments, binding to distinct, non-overlapping epitopes of Ara h 2, wherein the (exactly) three or four distinct antibodies, or antigen-binding fragments, are preferably selected from (i)-(iv) above.

In some embodiments, the medical use or treatment may comprise (combined) administration of (exactly) three distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above.

In some embodiments, the medical use or treatment may comprise (combined) administration of (exactly) three distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above;
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antibody, or an antigen-binding fragment thereof, comprising (a) the CDRH1, sequences of 7G6, or sequence variants thereof, as described above.

In some embodiments, the medical use or treatment may comprise (combined) administration of (exactly) three distinct antibodies, namely:
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above;
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

Preferably, the medical use or treatment may comprise (combined) administration of (exactly) three distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the medical use or treatment may comprise (combined) administration of (exactly) four distinct antibodies, namely:
(i) an antibody, or an antigen-binding fragment thereof, comprising (a) the CDRH1, sequences of 17H9, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above;
(iii) an antibody, or an antigen-binding fragment thereof, comprising (a) the CDRH1, CDRH2, CDRH3, CDRL1, CDRL2 and CDRL3 sequences and/or (b) the VH and VL sequences of 2F8, or sequence variants thereof, as described above; and
(iv) an antibody, or an antigen-binding fragment thereof, comprising (a) the CDRH1, CDRH2, CDRH3, CDRL1, CDRL2 and CDRL3 sequences and/or (b) the VH and VL sequences of 7G6, or sequence variants thereof, as described above.

In some embodiments, the medical use or treatment may comprise (combined) administration of (i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and (ii) an antibody, or an antigen-binding fragment thereof, comprising (a) the CDRH1, CDRH2, CDRH3, CDRL1, CDRL2 and CDRL3 sequences and/or (b) the VH and VL sequences of (wild-type) 7G6, or sequence variants thereof, as described herein, e.g. in Tables 1 and 2. Optionally, such a medical use or treatment may further comprise (combined) administration of an antibody, or an antigen-binding fragment thereof, including the CDR and/or VH/VL sequences of 15E3 (or a sequence variant thereof), and/or an antibody, or an antigen-binding fragment thereof, including the CDR and/or VH/VL sequences of 2F8 (or a sequence variant thereof).

In some embodiments, the medical use or treatment may comprise (combined) administration of (i) an antibody, or an antigen-binding fragment thereof, comprising (a) the CDRH1, CDRH2, CDRH3, CDRL1, CDRL2 and CDRL3 sequences and/or (b) the VH and VL sequences of 17H9, or sequence variants thereof, as described above; and (ii) an antibody, or an antigen-binding fragment thereof, comprising (a) the CDRH1, CDRH2, CDRH3, CDRL1, CDRL2 and CDRL3 sequences and/or (b) the VH and VL sequences of (wild-type) 2F8, or sequence variants thereof, as described herein, e.g. in Tables 1 and 2. Optionally, such a medical use or treatment may further comprise (combined) administration of an antibody, or an antigen-binding fragment thereof, including the CDR and/or VH/VL sequences of 15E3 (or a sequence variant thereof), and/or an antibody, or an antigen-binding fragment thereof, including the CDR and/or VH/VL sequences of 7G6 (or a sequence variant thereof).

In some embodiments, the medical use or treatment may comprise (combined) administration of (i) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and (ii) an antibody, or an antigen-binding fragment thereof, comprising (a) the CDRH1, CDRH2, CDRH3, CDRL1, CDRL2 and CDRL3 sequences and/or (b) the VH and VL sequences of (wild-type) 7G6, or sequence variants thereof, as described herein, e.g. in Tables 1 and 2. Optionally, such a medical use or treatment may further comprise (combined) administration of an antibody, or an antigen-binding fragment thereof, including the CDR and/or VH/VL sequences of 17H9 (or a sequence variant thereof), and/or an antibody, or an antigen-binding fragment thereof, including the CDR and/or VH/VL sequences of 2F8 (or a sequence variant thereof).

In some embodiments, the medical use or treatment may comprise (combined) administration of (i) an antibody, or an antigen-binding fragment thereof, comprising (a) the CDRH1, CDRH2, CDRH3, CDRL1, CDRL2 and CDRL3 sequences and/or (b) the VH and VL sequences of 15E3, or sequence variants thereof, as described above; and (ii) an antibody, or an antigen-binding fragment thereof, comprising (a) the CDRH1, CDRH2, CDRH3, CDRL1, CDRL2 and CDRL3 sequences and/or (b) the VH and VL sequences of (wild-type) 2F8, or sequence variants thereof, as described herein, e.g. in Tables 1 and 2. Optionally, such a the medical use or treatment may further comprise (combined) administration of an antibody, or an antigen-binding fragment thereof, including the CDR and/or VH/VL sequences of 17H9 (or a sequence variant thereof), and/or an antibody, or an antigen-binding fragment thereof, including the CDR and/or VH/VL sequences of 7G6 (or a sequence variant thereof).

The present invention also provides at least three distinct antibodies, or antigen-binding fragments thereof, binding to distinct, non-overlapping epitopes of Ara h 2 for use as a medicament, preferably for prophylaxis or treatment of a peanut allergy or a symptom of a peanut allergy, comprising (combined) administration of at least three of the following:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
(iii) an antibody, or an antigen-binding fragment thereof, comprising a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
(iv) an antibody, or an antigen-binding fragment thereof, comprising a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30.

Preferably, the medical use/treatment as described above comprises (combined) administration of at least three of the following:
(i) an antibody, or an antigen-binding fragment thereof, comprising a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
(iii) an antibody, or an antigen-binding fragment thereof, comprising a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
(iv) an antibody, or an antigen-binding fragment thereof, comprising a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30.

More preferably, the medical use/treatment as described above comprises (combined) administration of at least three of the following:
(i) an antibody, or an antigen-binding fragment thereof, comprising a VH having at least 70% identity to any one of SEQ ID NOs 7, 44 and 45; and a VL having at least 70% identity to any one of SEQ ID NOs 8, 46, 47, 48 and 49;
(ii) an antibody, or an antigen-binding fragment thereof, comprising a VH having at least 70% identity to any one of SEQ ID NOs 15, 37 and 38; and a VL having at least 70% identity to any one of SEQ ID NOs 16, 39 and 40;
(iii) an antibody, or an antigen-binding fragment thereof, comprising a VH having at least 70% identity to any one of SEQ ID NOs 23, 41, 50 and 53; and a VL having at least 70% identity to any one of SEQ ID NOs 24, 42 and 43;
(iv) an antibody, or an antigen-binding fragment thereof, comprising a VH having at least 70% identity to any one of SEQ ID NOs 31, 33 and 34; and a VL having at least 70% identity to any one of SEQ ID NOs 32, 35, 36 and 54.

Even more preferably, the medical use/treatment as described above comprises (combined) administration of at least three of the following:
(i) an antibody, or an antigen-binding fragment thereof, comprising a VH according to any one of SEQ ID NOs 7, 44 and 45; and a VL according to any one of SEQ ID NOs 8, 46, 47, 48 and 49;
(ii) an antibody, or an antigen-binding fragment thereof, comprising a VH according to any one of SEQ ID NOs 15, 37 and 38; and a VL according to any one of SEQ ID NOs 16, 39 and 40;
(iii) an antibody, or an antigen-binding fragment thereof, comprising a VH according to any one of SEQ ID NOs 23, 41, 50 and 53; and a VL according to any one of SEQ ID NOS 24, 42 and 43;
(iv) an antibody, or an antigen-binding fragment thereof, comprising a VH according to any one of SEQ ID NOs 31, 33 and 34; and a VL according to any one of SEQ ID NOs 32, 35, 36 and 54.

In some embodiments, the medical use/treatment as described above comprises (combined) administration of:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40; and
(iii) an antibody, or an antigen-binding fragment thereof, comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43.

In some embodiments, the medical use/treatment as described above comprises (combined) administration of:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(iii) an antibody, or an antigen-binding fragment thereof, comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antibody, or an antigen-binding fragment thereof, comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

In some embodiments, the medical use/treatment as described above comprises (combined) administration of:
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40;
(iii) an antibody, or an antigen-binding fragment thereof, comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antibody, or an antigen-binding fragment thereof, comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

Preferably, the medical use/treatment as described above comprises (combined) administration of:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40; and
(iv) an antibody, or an antigen-binding fragment thereof, comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

Preferably, the medical use/treatment as described above comprises (combined) administration of antibodies binding to four distinct, non-overlapping epitopes on Ara h 2. Accordingly, the medical use/treatment may comprise (combined) administration of the four distinct antibodies, or antigen-binding fragments, according to (i), (ii), (iii) and (iv).

Preferably, the medical use/treatment as described above comprises (combined) administration of:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to any one of SEQ ID NOS 7, 44 and 45; and a VL having at least 70% identity to any one of SEQ ID NOs 8, 46, 47, 48 and 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to any one of SEQ ID NOs 7, 44 and 45; and a VL according to any one of SEQ ID NOs 8, 46, 47, 48 and 49;
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NOS 15, 37 and 38; and a VL having at least 70% identity to any one of SEQ ID NOs 16, 39 and 40; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to any one of SEQ ID NOs 15, 37 and 38; and a VL according to any one of SEQ ID NOs 16, 39 and 40;
(iii) an antibody, or an antigen-binding fragment thereof, comprising:
   a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
   in some embodiments a VH having at least 70% identity to any one of SEQ ID NOS 23, 41, 50 and 53; and a VL having at least 70% identity to any one of SEQ ID NOS 24, 42 and 43; wherein the CDRH1 according to according to SEQ ID NO: 17, CDRH2 according to SEQ ID NO: 18, CDRH3 according to SEQ ID NO: 19 or 51, CDRL1 according to SEQ ID NO: 20, CDRL2 according to SEQ ID NO: 21, and CDRL3 according to SEQ ID NO: 22 are preferably maintained;
   such as a VH according to any one of SEQ ID NOs 23, 41, 50 and 53; and a VL according to any one of SEQ ID NOs 24, 42 and 43; and
(iv) an antibody, or an antigen-binding fragment thereof, comprising:
   a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30;
   in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30;
   in some embodiments a VH having at least 70% identity to any one of SEQ ID NOS 31, 33 and 34; and a VL having at least 70% identity to any one of SEQ ID NOs 32, 35, 36 and 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained;
   such as a VH according to any one of SEQ ID NOs 31, 33 and 34; and a VL according to any one of SEQ ID NOs 32, 35, 36 and 54.

More preferably, the medical use/treatment as described above comprises (combined) administration of:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6; in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14; in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
(iii) an antibody, or an antigen-binding fragment thereof, comprising a CDRH1 having at least 70% identity to according to SEQ ID NO: 17, a CDRH2 having at least 70% identity to SEQ ID NO: 18, a CDRH3 having at least 70% identity to SEQ ID NO: 51, a CDRL1 having at least 70% identity to SEQ ID NO: 20, a CDRL2 having at least 70% identity to SEQ ID NO: 21, and a CDRL3 having at least 70% identity to SEQ ID NO: 22; in particular a CDRH1 according to according to SEQ ID NO: 17, a CDRH2 according to SEQ ID NO: 18, a CDRH3 according to SEQ ID NO: 51, a CDRL1 according to SEQ ID NO: 20, a CDRL2 according to SEQ ID NO: 21, and a CDRL3 according to SEQ ID NO: 22; and
(iv) an antibody, or an antigen-binding fragment thereof, comprising a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30; in particular a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30.

Even more preferably, the medical use/treatment as described above comprises (combined) administration of:
(i) an antibody, or an antigen-binding fragment thereof, comprising a VH having at least 70% identity to SEQ ID NO: 45; and a VL having at least 70% identity to SEQ ID NO: 49; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained; such as a VH according to SEQ ID NO: 45, and a VL according to SEQ ID NO: 49;
(ii) an antibody, or an antigen-binding fragment thereof, comprising a VH having at least 70% identity to SEQ ID NO: 38; and a VL having at least 70% identity to SEQ ID NO: 39; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained; such as a VH according to SEQ ID NO: 38, and a VL according to SEQ ID NO: 39;
(iii) an antibody, or an antigen-binding fragment thereof, comprising a VH having at least 70% identity to SEQ ID NO: 53; and a VL having at least 70% identity to SEQ ID NO: 43; wherein the CDRH1 according to according to SEQ ID NO: 17, CDRH2 according to SEQ ID NO: 18, CDRH3 according to SEQ ID NO: 51, CDRL1 according to SEQ ID NO: 20, CDRL2 according to SEQ ID NO: 21, and CDRL3 according to SEQ ID NO: 22 are preferably maintained; such as a VH according to SEQ ID NO: 53, and a VL according to SEQ ID NO: 43; and
(iv) an antibody, or an antigen-binding fragment thereof, comprising a VH having at least 70% identity to SEQ ID NO: 34; and a VL having at least 70% identity to SEQ ID NO: 35 or 54; wherein the CDRH1 according SEQ ID NO: 25, CDRH2 according to SEQ ID NO: 26, CDRH3 according to SEQ ID NO: 27, CDRL1 according to SEQ ID NO: 28, CDRL2 according to SEQ ID NO: 29 or 52, and CDRL3 according to SEQ ID NO: 30 are preferably maintained; such as a VH according to SEQ ID NO: 34, and a VL according to SEQ ID NO: 35 or 54.

As described above, the medical use/treatment of the invention preferably comprises (combined) administration of antibodies binding to at least three, preferably four, distinct non-overlapping epitopes of Ara h 2. The antibodies, or the antigen-binding fragments thereof, which are administered (in combination), may be selected from the four different antibodies 17H9, 15E3, 2F8 and 7G6, or the variants thereof, as described above, in particular as shown in Tables 1 and 2. Preferably, therefore three or four (monospecific) antibodies as described herein, in particular selected from the four different antibodies 17H9, 15E3, 2F8 and 7G6, or the variants thereof, as described above, in particular as shown in Tables 1 and 2, may be administered (in combination). In some embodiments, multispecific antibodies, in particular as described herein, may (also) be administered.

Binding to three different epitopes of Ara h 2 may be obtained by (combined) administration of three distinct antigen-binding sites, in particular:
(i) three distinct monospecific antibodies, or antigen-binding fragments thereof, binding to three distinct epitopes of Ara h 2, in particular selected from 17H9, 15E3, 2F8 and 7G6, or the variants thereof, as described above, in particular as shown in Tables 1 and 2;
(ii) (1) a bispecific antibody, or a bispecific antigen-binding fragment, in particular having two specificities selected from 17H9, 15E3, 2F8 and 7G6, or the variants thereof, as described above, in particular as shown in Tables 1 and 2, and (2) an additional monospecific antibody, in particular selected from 17H9, 15E3, 2F8 and 7G6, or the variants thereof, as described above, in particular as shown in Tables 1 and 2, binding to another epitope on Ara h 2; or
(iii) a trispecific antibody, or a trispecific antigen-binding fragment, in particular having three specificities selected from 17H9, 15E3, 2F8 and 7G6, or the variants thereof, as described above, in particular as shown in Tables 1 and 2.

Binding to four different epitopes of Ara h 2 may be obtained by (combined) administration of four distinct antigen-binding sites, in particular:
(i) four distinct monospecific antibodies, or antigen-binding fragments thereof, binding to three distinct epitopes of Ara h 2, in particular selected from 17H9, 15E3, 2F8 and 7G6, or the variants thereof, as described above, in particular as shown in Tables 1 and 2;
(ii) (1) a bispecific antibody, or a bispecific antigen-binding fragment, in particular having two specificities selected from 17H9, 15E3, 2F8 and 7G6, or the variants thereof, as described above, in particular as shown in Tables 1 and 2, and (2) two additional monospecific antibodies, in particular selected from 17H9, 15E3, 2F8 and 7G6, or the variants thereof, as described above, in particular as shown in Tables 1 and 2, binding to further distinct epitopes on Ara h 2;
(iii) two distinct bispecific antibodies, or a bispecific antigen-binding fragments, in particular wherein each of the bispecific antibodies, or a bispecific antigen-binding fragments, has two distinct specificities selected from 17H9, 15E3, 2F8 and 7G6, or the variants thereof, as described above, in particular as shown in Tables 1 and 2;
(iv) (1) a trispecific antibody, or a trispecific antigen-binding fragment, in particular having three specificities selected from 17H9, 15E3, 2F8 and 7G6, or the variants thereof, as described above, in particular as shown in Tables 1 and 2, and (2) an additional monospecific antibody, in particular selected from 17H9, 15E3, 2F8 and 7G6, or the variants thereof, as described above, in particular as shown in Tables 1 and 2, binding to another epitope on Ara h 2; or
(v) a tetraspecific antibody, or a tetraspecific antigen-binding fragment, in particular having the four specificities of 17H9, 15E3, 2F8 and 7G6, or the variants thereof, as described above, in particular as shown in Tables 1 and 2.

Alternatively, or additionally, at least one additional agent useful for treating peanut allergy may be administered (in combination) with the antibodies, or antigen-binding fragments, as described herein. The additional agent useful for treating peanut allergy may be selected from the group comprising: a β-adrenergic agonist (e.g. epinephrine), antihistamine, a corticosteroid, an anti-IgE antibody, an anti-IgE antibody binding fragment, a peptide vaccine and further antibodies capable of binding to a peanut allergen. In some embodiments, the additional agent is a β-adrenergic agonist, such as epinephrine.

In some embodiments, a peanut allergen may be administered (in combination) with the antibodies, or antigen-binding fragments, as described herein. The peanut allergen may be untreated or treated peanut, such as peanut powder, roasted peanut or peanut butter. In some embodiments, the peanut allergen may be a commercially available product, such as Palforzia (defatted powder of peanuts). The peanut allergen may be a peanut protein, such as Ara h 1, Ara h 2, Ara h 3, Ara h 4, Ara h 5, Ara h 6/7, Ara h 8, Ara h 9 and Ara h 10/11. In a specific embodiment the peanut protein may be in particular selected from Ara h 2, Ara h 3 and Ara h 6, or a combination thereof. The peanut allergen may be of peanut origin, recombinantly expressed or is a synthetic peanut peptide.

In some embodiments, the anti-peanut allergen antibodies, or antigen-binding fragments thereof, as described herein, may be pre-incubated with the peanut allergen and may be administered as a mixture to a subject. In other embodiments, the antibody, or the antigen-binding fragment thereof, or the composition comprising said antibody is administered before or during a desensitization procedure with a peanut allergen.

Combining administration of a peanut allergen with the antibodies of the invention increases the safety of administering the allergen (e.g. in desensitization). Furthermore, without being bound to any theory, the present inventors assume that the combination of antibodies with the respective allergen (targeted by the antibodies), i.e. the combination of passive (antibodies) and active (allergen) immunization, has a synergistic effect.

### Further Uses

Antibodies and fragments thereof as described herein may also be used for the (in-vitro) diagnosis of a peanut allergy. Methods of diagnosis may include contacting an antibody with a sample. Such samples may be isolated from a subject, for example an isolated blood sample, such as whole blood, plasma or serum. The methods of diagnosis may also include the detection of an antigen/antibody complex, in particular following the contacting of an antibody with a sample. Furthermore, it may be tested, whether the sample contains antibodies competing with the antibodies as described herein, e.g. for allergen binding. This is typically performed in vitro, i.e. without any contact to the human or animal body. Examples of analytical methods are well-known to the person skilled in the art and include immunoassays such as flow cytometry, dot or slot blots, Western blots, ELISA (enzyme-linked immunosorbent assay), e.g. for cross-competition, immunohistochemistry and immunoprecipitation followed by SDS-PAGE immunocytochemistry. Accordingly, the diagnosis may be performed in vitro, for example by using an isolated sample as described above (and an in vitro analysis step as described above).

The present invention also provides a method of detecting whether a sample comprises a peanut allergen, in particular Ara h 2, Ara h 3 and/or Ara h 6. Such a method may comprise the following steps:
a. contacting the sample with the antibody described herein under conditions permissive to produce an antibody/antigen complex; and
b. detecting the presence of the antibody/antigen complex.

Thereby, the presence of detectable antibody/antigen complex may be indicative that the sample may contain a peanut allergen, in particular Ara h 2, Ara h 3 and/or Ara h 6.

In some embodiments, in step (b) the amount of the antigen/antibody complex may be determined (e.g., measured) in the test sample. Thereafter, said amount may be compared to a control.

Accordingly, the antibody of the present invention, or an antigen-binding fragment thereof, may be used in an (in vitro) method for detecting a peanut allergen, in particular Ara h 2, Ara h 3 and/or Ara h 6. Likewise, the antibody of the present invention, or an antigen-binding fragment thereof, may be used in an (in vitro) method for binding a peanut allergen, in particular Ara h 2, Ara h 3 and/or Ara h 6. For detecting a peanut allergen, in particular Ara h 2, Ara h 3 and/or Ara h 6, the antibody may be brought in contact with a (isolated) sample (i.e., a sample to be tested for the presence of the antigen). By the specific binding of the antibody to its antigen (a peanut allergen, in particular Ara h 2, Ara h 3 and/or Ara h 6), an antibody/antigen complex is formed, which can be easily detected by methods known in the art.

Such a detection method may be used for testing samples (e.g., production/manufacture), such as food, cosmetics or medication samples. Accordingly, antibodies, antibody fragment, or variants thereof, as described in the present invention may also be used in a non-therapeutic/non-diagnostic context, e.g. in development or manufacture of various products.

Furthermore, the detection method may be used for testing vaccine samples (e.g. for use in desensitization), whether they contain the major peanut allergens Ara h 2, Ara h 3 and/or Ara h 6. This may be useful in the development and/or manufacture of such vaccines. Accordingly, the present invention therefore also provides the use of the antibodies as described herein, or an antigen-binding fragment thereof, for testing immunogenic compositions/vaccines, in particular of an immunogenic composition/vaccine comprising a a peanut allergen. To this end, the antibody may be brought in contact with the immunogenic composition/vaccine, e.g. to test for antibody/antigen complexes. Accordingly, the present invention also provides a method for testing immunogenic compositions (vaccines) based on peanut allergens, wherein the immunogenic composition/vaccine is contacted with the antibody, or an antigen-binding fragment thereof, and, optionally, the presence of antibody/antigen complexes is determined. Furthermore, the present invention also encompasses the use of the antibody of the present invention, or an antigen-binding fragment thereof, for monitoring the quality of immunogenic compositions/vaccines based on peanut allergens by checking whether the immunogenic composition/vaccine contains the desired antigen, e.g. Ara h 2, Ara h 3 and/or Ara h 6.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows for Example 2 the SPR sensogram for antibodies 15E3, 2F8, 7G6 and 17H9 binding to nAra h 2. 15E3 (Fab) was immobilized and nAra h 2, 2F8, 7G6 and 17H9 (as Fabs) were injected sequentially as indicated in the Figure.

FIG. 2 shows for Example 2 the BLI sensogram for antibodies 15E3, 2F8, 7G6 and 17H9 binding to nAra h 2. 15E3 (IgG) was immobilized and nAra h 2, 2F8, 7G6 and 17H9 (as as human-mouse IgG chimera) were injected sequentially as indicated in the Figure.

FIG. 3 shows for Example 3 that a cocktail (“MY006-4”) of antibodies 2F8, 7G6, 17H9 and 15E3 inhibits patient IgE binding to nAra h 2 (mean inhibition=94%) and peanut extract (mean inhibition=78%) in a population of 37 peanut allergic patients, as measured by ELISA.

FIG. 4 shows for Example 3 that titrated cocktail (“MY006-4”) of antibodies 2F8, 7G6, 17H9 and 15E3 inhibits 80% of IgE binding to peanut extract (“PE”), of IgE's derived from a pool containing plasma from 91 peanut allergic patients (IC50: 751.8 pM).

FIG. 5 shows for Example 4 that pre-incubation of peanut extract with the cocktail of antibodies 2F8, 7G6, 17H9 and 15E3 decreased basophil degranulation. A: Bars indicate activation of basophils expressed as % CD63+ cells (mean±s.d, n=2). Black bars: no addition of antibody. Data from 1 representative patient. Statistics done by One way Anova followed by Dunnett's test comparing each row to 0.1 ng/ml PE. B: Dose titration of peanut extract with or without the cocktail of antibodies 2F8, 7G6, 17H9 and 15E3. Shown is mean±s.d, n=2, data from one representative patient.

FIG. 6 shows for Example 5 (A) the experimental schedule and (B) that the cocktail (“MY006-4”) of antibodies 2F8, 7G6, 17H9 and 15E3 prevents anaphylaxis in a passive cutaneous anaphylaxis (PCA) mouse model (huFcaRI transgenic mice (IgE/FcεRI humanized mouse line, Genoway)).

FIG. 7 shows for Example 7 that a cocktail (“MY006-4-cocktail”) of antibodies 2F8, 7G6, 17H9 and 15E3 as well as multispecific constructs “MY006-tetraspecific”, “MY006-trispecific” and “2× MY006-bispecific” inhibit patient's (n=16) IgE binding to peanut allergens.

FIG. 8 shows for Example 8 that a cocktail (“MY006-4-cocktail”) of antibodies 2F8, 7G6, 17H9 and 15E3 as well as multispecific constructs “MY006-tetraspecific”, “MY006-trispecific” and “2× MY006-bispecific” inhibit binding of IgEs derived from a pool containing plasma from 90 peanut allergic patients to peanut extract.

FIG. 9 shows for Example 9 that a cocktail (“MY006-4-IgG4-cocktail”) of antibodies 2F8, 7G6, 17H9 and 15E3 as well as multispecific constructs “MY006-tetraspecific” and “2× MY006-bispecific” inhibit basophil degranulation (allergen-mediated release of leukotrienes (sLT)) to the same extent. Shown is data from 5 experiments, mean±SEM.

FIG. 10 shows for Example 10 that a tetraspecific antibody (“MY006-tetraspecific”) with the specificities of parental antibodies 2F8, 7G6, 17H9 and 15E3, a cocktail of two bi-specific antibodies (with the specificities of (i) 17H9 and 7G6 and (ii) 15E3 and 2F8; “2×MY006-bi-specific”), or a single bispecific antibody (with the specificities of 17H9 and 7G6; “MY006-bi-specific”) prevent anaphylaxis in a passive cutaneous anaphylaxis (PCA) mouse model (huFcaRI transgenic mice (IgE/FcεRI humanized mouse line, Genoway). (A) Experimental schedule; (B) Amount of Evans blue/ear for each group indicating the anaphylactic reaction. Statistics done by One way Anova followed by Dunnett's test comparing each row to isotype control.

FIG. 11 shows for Example 11 that reversion of somatic mutations in the framework region to germline residues did not affect the functionality of the antibodies, as they were still able to bind to Ara h 2.

FIG. 12 shows for Example 12 ion exchange data for wildtype 2F8 (including CDRH3 according to SEQ ID NO: 19) and a 2F8 CDRH3 variant, wherein the wildtype CDRH3 was replaced with SEQ ID NO: 51 (three mutations). While several peaks were observed for 2F8, only a single peak is present for the 2F8 CDRH3 variant, indicating a homogenous product.

FIG. 13 shows for Example 12 binding data for 2F8 and the 2F8 CDRH3 variant to the two antigens Ara h 2 and Ara h 6. No differences in binding were found despite the three mutations in the CDRH3.

FIG. 14 shows for Example 13 the results of the insulin/DNA-binding ELISA (A) for the different antibodies 15E3, 17H9, 7G6 and 2F8 and (B) for the 7G6 CDRL2 variant, wherein the wildtype CDRL2 was replaced with SEQ ID NO: 52 compared to wild-type 7G6. Non-specific interactions were reduced in the 7G6 CDRL2 variant, wherein the wildtype CDRL2 was replaced with SEQ ID NO: 52.

FIG. 15 shows for Example 13 binding data for 7G6 and the 7G6 CDRL2 variant to peanut extract (PE). No differences in binding were found despite the mutation in the CDRL2.

FIG. 16 shows for Example 14 that a cocktail (“MY006-4-cocktail”) of antibodies 2F8, 7G6, 17H9 and 15E3 prevents anaphylaxis in a humanized mouse model of peanut allergy. (A) Treatment schedule; (B) body temperature; and (C) allergy scoring for mice (n=4 per group) treated either with the antibody cocktail (“MY006-4-cocktail”) or with isotype control.

FIG. 17 shows for Example 15 the binding affinities as determined by ELISA of Group 1 antibody 7G6 (A), Group 3 antibodies 12G10, 37D5 and 17H9 (B), Group 2 antibodies 32B10 and 2F8 (C), and Group 4 antibodies 4B2 and 15E3 (D) to nAra h 1, nAra h 2, nAra h 3 and nAra h 6.

FIG. 18 demonstrates maintenance of the binding specificity of a fully rabbit antibody containing 2F8_rb_IGHV1S7_J4+IGKV1S21_JK1-2 (FIG. 18A) to antigens Ara h2 and Ara h6, respectively (negative control: BSA, positive control peanut (“pe”: Peanut extract). FIG. 18B shows the variable framework region of the heavy chain and its wt counterpart sequence (2F8_Hg: SEQ ID NO: 119) (upper alignment) and the variable framework region of the light chain IGKV1S21_JK1-2 and its wt counterpart sequence (2F8_Lwt: SEQ ID NO: 120) (lower alignment). The CDR sequences of the heavy chain (CDRH1, CDRH2 and CDRH3) and the light chain (CDRL1, CDRL2 and CDRL3) are indicated as grey boxes.

FIG. 19 demonstrates maintenance of the binding specificity of fully rabbit antibodies containing constructs 7G6_rb_IGHV1S47_J3+IGKV1S19_JK1-2 (FIG. 19A), 7G6_rb_IGHV1S47_J3+IGKV1S19_JK1-3 (FIG. 19B), and 7G6_rb_IGHV1S47_J3+K66N_IGKV1S19_JK1-2 (FIG. 19C) to antigens Ara h2 and Ara h6, respectively (negative control: BSA, positive control peanut (“pe”). FIG. 19D shows the variable framework region of the heavy chain and its wt counterpart sequence (7G6_Hwt: SEQ ID NO: 121) (upper alignment) and the variable framework regions of the three light chains IGKV1S19_JK1-2, IGKV1S19_JK1-3, and K66N_IGKV1S19_JK1-2 and their wt counterpart sequence (7G6_Lwt: SEQ ID NO: 122) and a consensus sequence (in bold) (lower alignment). The CDR sequences of the heavy chain (CDRH1, CDRH2 and CDRH3) and the light chain (CDRL1, CDRL2 and CDRL3) are indicated as grey boxes.

FIG. 20 demonstrates maintenance of the binding specificity of fully rabbit antibodies containing constructs 17H9_rb_IGHV1S7_J3+IGKV1S34_JK1-2 (FIG. 20A) and 17H9_rb_IGHV1S7_J3+IGKV1S34_JK1-5 (FIG. 20B) to antigens Ara h2 and Ara h3, respectively (negative control: BSA, positive control peanut (“pe”). FIG. 20C shows the variable framework region of the heavy chain and its wt counterpart sequence (17H9_Hwt: SEQ ID NO: 123) (upper alignment) and the variable framework regions of the two light chains IGKV1S34_JK1-2 and IGKV1S34_JK1-5 and their wt counterpart sequence (17H9_Lwt: SEQ ID NO: 124) and a consensus sequence (in bold) (lower alignment). The CDR sequences of the heavy chain (CDRH1, CDRH2 and CDRH3) and the light chain (CDRL1, CDRL2 and CDRL3) are indicated as grey boxes.

FIG. 21 demonstrates maintenance of the binding specificity of a fully rabbit antibody containing 15E3_rb_IGHV1S8_J5+IGLV2S2_JL5 to antigens Ara h2 and Ara h6 (FIG. 21A), respectively (negative control: BSA, positive control peanut (“pe”). FIG. 21B shows the variable framework region of the heavy chain and its wt counterpart sequence (15E3_Hwt: SEQ ID NO: 125) (upper alignment) and the variable framework region of the light chain and its wt counterpart sequence (15E_3_Lwt: SEQ ID NO: 126) (lower alignment). The CDR sequences of the heavy chain (CDRH1, CDRH2 and CDRH3) and the light chain (CDRL1, CDRL2 and CDRL3) are indicated as grey boxes.

FIG. 22 demonstrates maintenance of the binding specificity of antibodies (7 antibodies: 7G6_G1, 7G6_G2, 7G6_G4, 7G6_G5, 7G6_G6, 7G6_G7 and 7G6_G8) having 7G6 CDR sequences and containing distinct human variable heavy chain and/or light chain framework regions (see Table 14) to antigen Ara h2 (FIG. 22A). The results for the antibody containing the corresponding wt sequence (7G6_wt) are shown as well. FIG. 22B presents a sequence alignment of the human light/heavy chain variable framework regions of the wt counterpart sequence (7G6_wt; SEQ ID NO: 127) and of the sequences of constructs7G6_G1, 7G6_G2, 7G6_G4, 7G6_G5, 7G6_G6, 7G6_G7 and 7G6_G8. The CDR sequences are indicated by grey boxes.

FIG. 23 demonstrates maintenance of the binding specificity of an antibody having 15E3 CDR sequences and containing the human VL (lambda) variable framework region sequence encoded by VL gene IGLV1-51 and the human VH variable framework region sequence encoded by VH gene IGHV3-7 (15E3_G4) to antigen Ara h2 (FIG. 23A). The results for the antibody containing the corresponding wt counterpart sequence (15E3_wt) are shown as well. FIG. 23B presents a sequence alignment of the human light/heavy chain variable framework regions of the wt counterpart sequence (15E3_wt; SEQ ID NO: 128) and of the sequence of construct 15E3_G4. The CDR sequences are indicated by grey boxes.

FIG. 24 shows the result of cytokine secretion (pg/mL) measured upon in vitro stimulation of PBMCs of a peanut allergic patient by either the antibody cocktail “MY006-4” in combination with the peanut allergen Ara h2 or by Ara h2 alone. FIG. 24A presents the measured levels of secreted IL-10. FIG. 24B presents the measured levels of secreted IL-13.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Antibodies 2F8, 7G6, 17H9 and 15E3 specifically bind to Ara h 2, Ara h 3 and/or Ara h 6

Fully human antibodies 2F8 and 7G6 were previously described in WO 2018/234383 A1. Fully human antibodies 17H9 and 15E3 were isolated and cloned in a similar manner, i.e. by the method as described in detail in WO 2018/234383 A1. Briefly, as starting material for the cloning of fully human antibodies, human lymphocytes were obtained from peripheral blood of voluntary allergic patients. Antibodies specific to major peanut allergens were isolated by molecular cloning of immunoglobulin genes obtained from single-cell sorted cells derived from short term oligoclonal cultures of activated memory B cells producing the antibodies of interest. Molecular cloning of human antibodies specific to peanut allergens was carried out according to Huang J, Doria-Rose N A, Longo N S, Laub L, Lin C L, Turk E, Kang B H, Migueles S A, Bailer R T, Mascola J R, Connors M. Isolation of human monoclonal antibodies from peripheral blood B cells. Nat Protoc. 2013 October; 8 (10): 1907-15. doi: 10.1038/nprot.2013.117, as described in detail in WO 2018/234383 A1. CDR and VH/VL sequences (SEQ ID NOs) of fully human antibodies 17H9, 15E3, 2F8 and 7G6 are shown in Table 4 below:

TABLE 4
SEQ ID NOs for CDR and VH/VL sequences
of antibodies 17H9, 15E3, 2F8 and 7G6

| Antibody | CDRH1 | CDRH2 | CDRH3 | CDRL1 | CDRL2 | CDRL3 | VH | VL |
|---|---|---|---|---|---|---|---|---|
| 17H9 | 1 | 2 | 3 | 4 | 5 | 6 | 7 | 8 |
| 15E3 | 9 | 10 | 11 | 12 | 13 | 14 | 15 | 16 |
| 2F8 | 17 | 18 | 19 | 20 | 21 | 22 | 23 | 24 |
| 7G6 | 25 | 26 | 27 | 28 | 29 | 30 | 31 | 32 |

Monoclonal antibodies 2F8, 7G6, 17H9 and 15E3 were tested for their specificity against the major peanut allergens Ara h 1, 2, 3, and 6 by ELISA (enzyme linked immunosorbent assay). Briefly, ELISA plates were coated with respective Ara h proteins (Arah1: 0.5 ug/ml, Arah2: 0.01 ug/ml, Arah3: 0.1 ug/ml, Arah6: 0.05 ug/ml) overnight at 4° C. Afterwards, plates were blocked with BSA (2% in PBS) for 1 hour at room temperature. Antibody titration series starting from 3 ug/ml was prepared, added to the single ELISA plate wells and incubated for 2 h at room temperature. The read-out was performed by adding anti-hu-IgG-HRP (anti-human-IgG horseradish peroxidase; Jackson ImmunoResearch, West Grove, PA, USA) for 1 h at room temperature. Tetramethylbenzidine substrate solution (TMB, Sigma-Aldrich Chemie GmbH, Buchs, Switzerland) was added, then after 5 minutes of hydrolysis time, the reaction was stopped by the addition of 1 M H₂SO₄ and the absorbances were read at 450 nm.

Results are shown in Table 5 below:

TABLE 5
Binding data [EC50] obtained by ELISA [ng/ml]

| Antibody | Ara h 2 | Ara h 3 | Ara h 6 |
|---|---|---|---|
| 2F8 | 3.305 | | 2.461 |
| 7G6 | 0.6294 | | 1.001 |
| 17H9 | 2.204 | 7.376 | |
| 15E3 | 3.778 | | 1.735 |

These binding data were confirmed by surface plasmon resonance (SPR) experiments (data not shown).

These data show that all tested antibodies 2F8, 7G6, 17H9 and 15E3 bind specifically to Ara h 2. In addition, 2F8, 7G6 and 15E3 bind specifically to Ara h 6, while 17H9 additionally binds specifically to Ara h 3.

### Example 2: Antibodies 2F8, 7G6, 17H9 and 15E3 Bind to Distinct Non-Overlapping Epitopes on Ara h 2

To determine whether antibodies 2F8, 7G6, 17H9 and 15E3 can bind simultaneously to nAra h 2, SPR sequential binding of the respective Fab fragments of the respective antibodies was performed. The antigen nAra h 2 is a mixture of two different isoforms (SEQ ID NO: 55 and SEQ ID NO: 56). Briefly, SPR was studied at 25° C. using a real-time biosensor surface plasmon resonance assay (BIACORE™ 8K), using 10 mM HEPES pH 7.4, 150 mM NaCl, 3 mM EDTA and 0.005% Tween-20 as running buffer. First, 15E3 (Fab) was immobilized on the surface of a CM5 sensor chip at a concentrations of 50 nM through standard amine coupling, then nAra h 2 (100 nM) was applied, followed by sequential injection of 2F8 (Fab; 300 nM), 7G6 (Fab; 50 nM) and 17H9 (Fab; 300 nM).

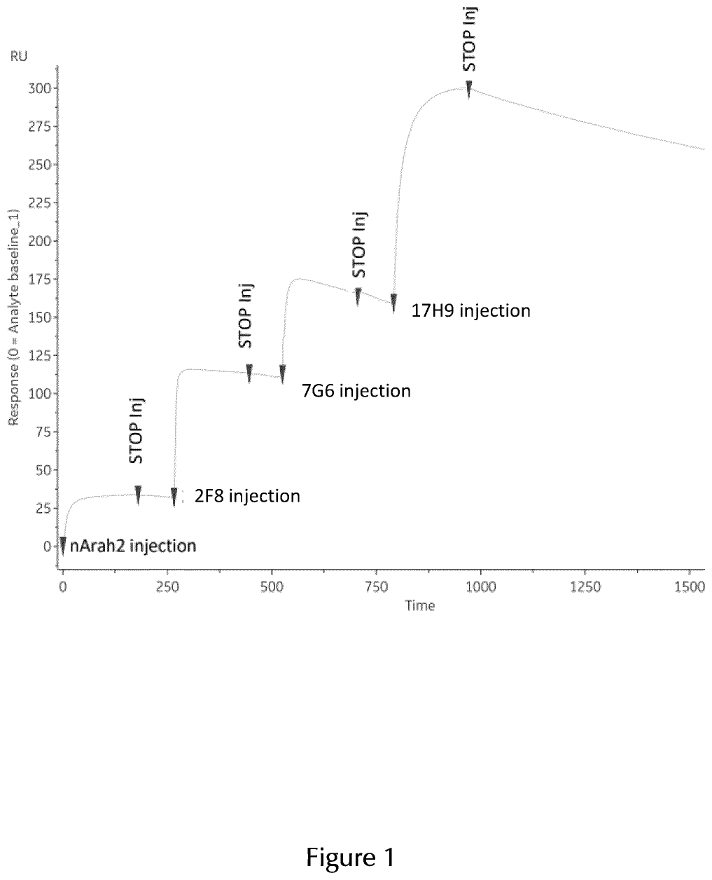

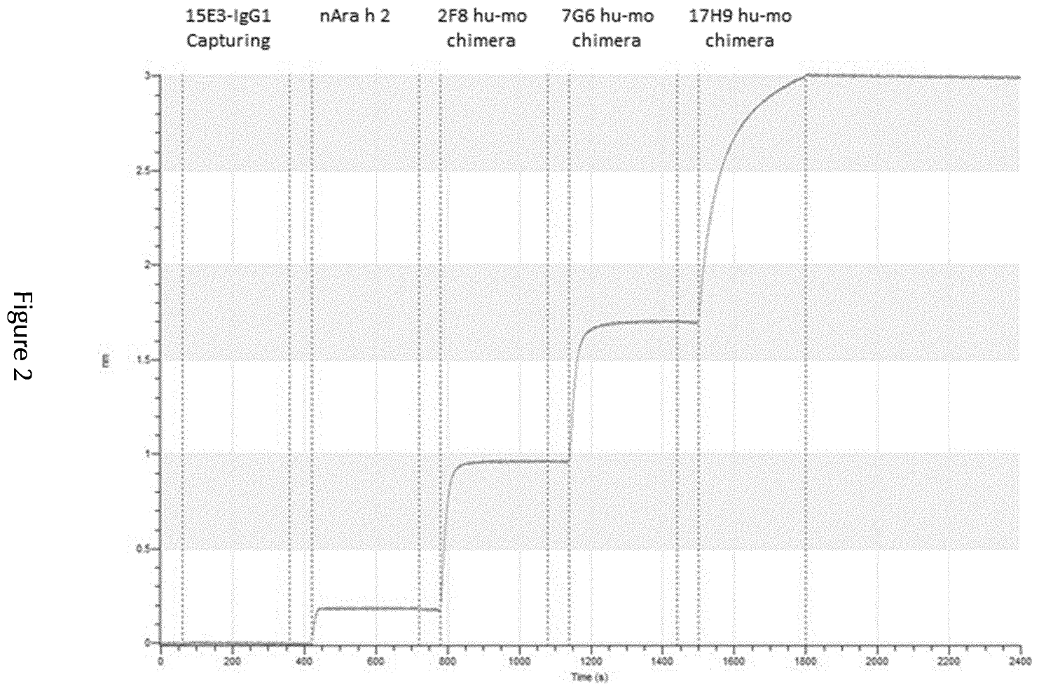

Results are shown in FIG. 1. The sensogram strongly suggests that all the Fabs can bind at the same time to a single Ara h2 molecule. Additionally, the change in response units (RU) when adding 17H9 was 2-3 times higher than compared to 2F8 or 7G6, suggesting that more than one 17H9 Fab molecule can bind to one nAra h 2.

Next, biolayer interferometry (BLI; Forte Bio Octet RED96e; Running buffer: 1×PBS) was used to determine whether all antibodies can bind to Ara h 2 simultaneously, when IgG molecules of the respective antibodies were used. First, 15E3-human IgG1 was immobilized on a human Fc binding BLI sensor surface (Anti-human IgG (AHQ) Biosensors (Fc-specific)) at a concentration of 5 ug/ml (33 nM). Then nAra h 2 (100 nM) was applied, followed by sequential injection of 2F8, 7G6 and 17H9; each as human-mouse chimera, wherein the human Fc was exchanged to murine Fc (CH1-hinge from human IgG1 was fused to murine Fc (CH2-CH3) to avoid binding to BLI sensor surface; each at a concentration of 10 ug/ml (66 nM).

Results are shown in FIG. 2. The sensogram strongly suggests that all tested antibodies 2F8, 7G6, 17H9 and 15E3 can bind at the same time to a single Ara h 2 molecule also as IgGs. Additionally, the change in nm shift when adding 17H9 was 2-3 times higher than compared to 2F8 or 7G6, suggesting that more than one 17H9 IgG molecule can bind to one nAra h 2.

In summary, the BLI data confirmed the results obtained for Fabs with SPR for IgG antibodies, namely that 2F8, 7G6, 17H9 and 15E3 can bind simultaneously to a single Ara h 2 molecule. Accordingly, these data demonstrate that 2F8, 7G6, 17H9 and 15E3 bind to distinct, non-overlapping epitopes on Ara h 2, both as Fab fragments and full IgG molecules.

### Example 3: Cocktail of Antibodies 2F8, 7G6, 17H9 and 15E3 Inhibits Patient IgE Binding to Peanut Allergens

As antibodies 2F8, 7G6, 17H9 and 15E3 can bind simultaneously to Ara h 2, they may act synergistically when combined, e.g. in a cocktail. Accordingly, it was investigated whether a cocktail comprising antibodies 2F8, 7G6, 17H9 and 15E3 inhibits patient IgE binding to peanut allergens (Peanut extract/nAra h 2). Briefly, ELISA plates were coated with anti-IgE (10 ug/ml), overnight at 4° C. and plasma IgE from individual peanut allergic patients (n=37) and plasma pool (mix of 91 plasma of allergic patients) were captured (2 h incubation at room temperature). At the same time, biotinylated allergens (1.12 nM peanut extract/1.2 nM Ara h 2) were pre-incubated with the antibody cocktail comprising 2F8, 7G6, 17H9 and 15E3 or isotype control (80 nM for single patients, and titration series starting at 80 nM for plasma pool) at least 1 h at room temperature. Allergen binding to IgE in presence of antibodies was measured using Streptavidin-HRP as read-out. % IgE binding inhibition was calculated as: % inhibition=100-(OD450_{Ab}/OD450_{w/o Ab}*100) and normalized to assay internal control, defined by the competition between MY006-IgE captured on the plate and its IgG counterpart.

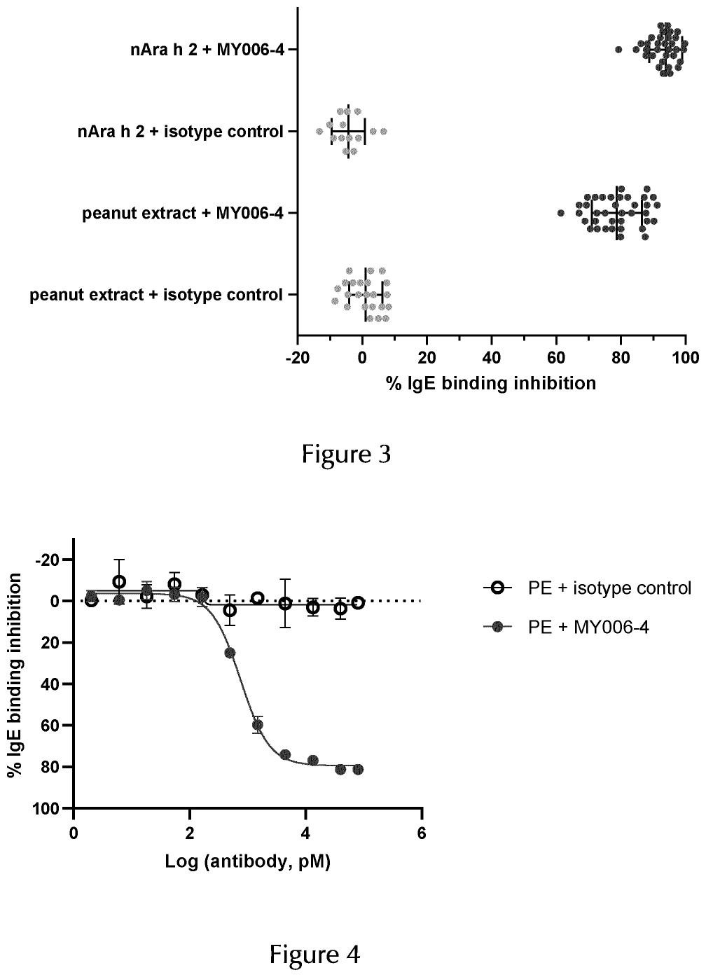

Results are shown in FIGS. 3 and 4. In FIG. 3, the % IgE binding inhibition of the MY006 cocktail and an isotype control was plotted for each single patient plasma. FIG. 4 depicts the capacity of MY006 cocktail and its respective isotype control to inhibit binding of patients' IgE (plasma pool) to peanut extract (PE) in a dose-dependent manner. As shown in FIG. 3, the cocktail comprising antibodies 2F8, 7G6, 17H9 and 15E3 inhibits patient IgE binding to nAra h 2 (mean inhibition=94%) and peanut extract (mean inhibition=79%) in a population of 37 peanut allergic patients. Titrated cocktail comprising antibodies 2F8, 7G6, 17H9 and 15E3 inhibits 80% of IgE binding to peanut extract of IgEs derived from a pool containing plasma from 91 peanut allergic patients (IC50: 751.8 pM, FIG. 4).

### Example 4: Cocktail of Antibodies 2F8, 7G6, 17H9 and 15E3 Inhibits Allergen-Mediated Activation of Basophils Derived from Allergic Patients

Next, it was investigated whether the cocktail of antibodies 2F8, 7G6, 17H9 and 15E3 can inhibit allergen-mediated activation of basophils derived from allergic patients.

To this end, a whole blood basophil activation test was performed. Briefly, heparinized whole blood from peanut allergic donors was either left untreated or stimulated for 30 minutes at 37° C. with serial dilutions of peanut extract in absence or in the presence of the cocktail of antibodies (80 nM) 2F8, 7G6, 17H9 and 15E3 (pre-incubation peanut extract/antibodies performed at room temperature for at least 1 h). Before erythrocyte lysis, cells were stained with CCR3-PE and CD63-FITC. Basophils were gated as SSClow, CCR3high lymphocytes and activation was quantified using % of CD63+, CCR3high cells using FACS Calibur (Becton Dickinson A G, Allschwil, Switzerland). The flow cytometry data was analyzed using FlowJo software (TreeStar, Ashland, Ore).

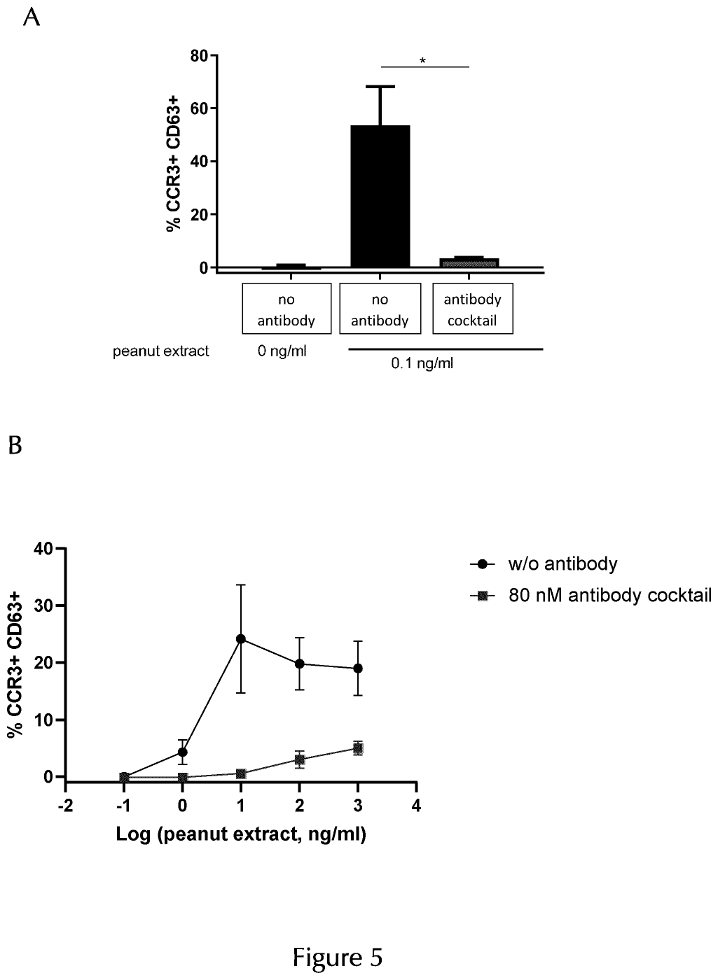

Results are shown in FIG. 5 with activated basophils (CD63+) at given peanut extract concentration (0.1 ng/ml), example of 1 patient (A) and activated basophils (CD63+) over peanut dose response curve, example of 1 patient (B). In summary, these data show that pre-incubation of peanut extract with the antibody cocktail decreased basophil degranulation.

### Example 5: Cocktail of Antibodies 2F8, 7G6, 17H9 and 15E3 Prevents Allergy in a Humanized Mouse Model of Peanut Allergy

To investigate the effects of the cocktail of antibodies 2F8, 7G6, 17H9 and 15E3 in vivo, a passive cutaneous anaphylaxis (PCA) mouse model (huFcaRI transgenic mice (IgE/FcεRI humanized mouse line, Genoway)) was used.

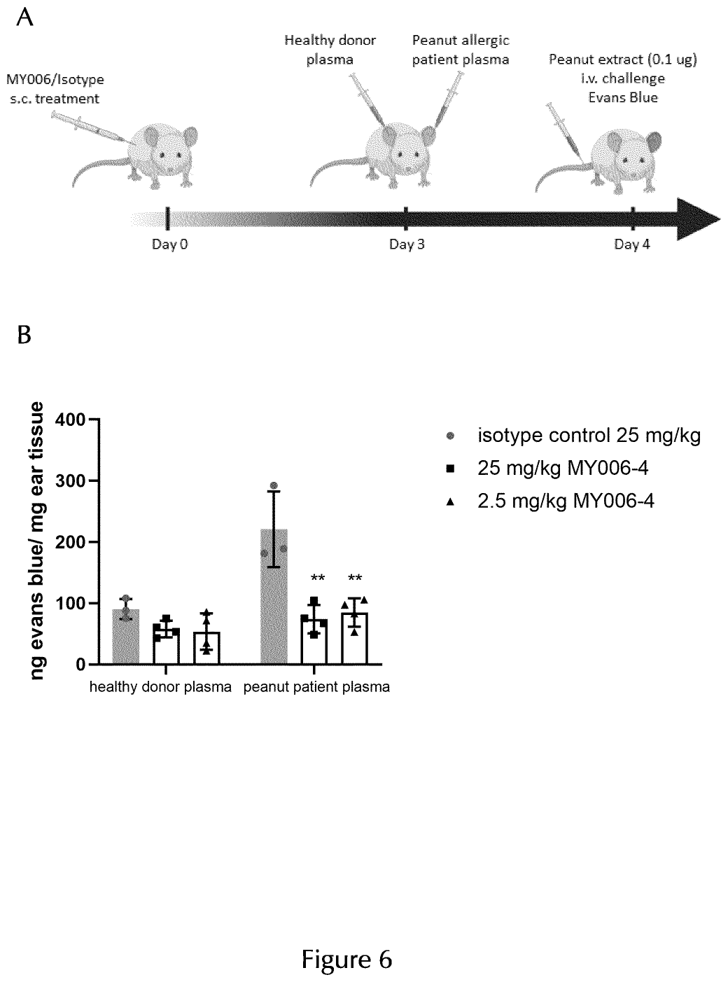

To study the effects of the cocktail of antibodies 2F8, 7G6, 17H9 and 15E3, huFcεRI transgenic mice were administered subcutaneously with the antibody cocktail at a dose of 2.5 or 25 mg/kg or with isotype control. Three days later, ears of transgenic mice were intradermally (i.d.) sensitized by injection of a pool of human plasma. In the right ear, 15 μl of plasma pool from peanut allergic individuals was injected, whilst the left ear was treated with a plasma pool from healthy individuals (internal experimental control). 24 hours later, animals were challenged by injection with peanut extract (PE) i.v (0.1 μg) along with Evans blue dye (0.5%). The experimental schedule is shown in FIG. 6A. Thirty minutes after challenge, mice were sacrificed and ears were collected to measure ear thickness. Afterwards, ears were placed in a formamide solution to extract and quantify the dye. Local allergic reaction in the sensitized ears was monitored by the amount of extravasation of the Evans blue staining. The amount of extravasated Evans blue was measured by optical absorption at 620 nm and normalized to the weight of the ear tissue collected (Evans blue ng/mg ear tissue). Statistics were done by One-way Anova followed by Dunnett's test comparing each column to isotype control.

Results are shown in FIG. 6B. Upon sensitization with peanut patient plasma, the data show an allergic reaction in the group treated with isotype control, while no allergic reaction was apparent in the groups treated with the cocktail of antibodies 2F8, 7G6, 17H9 and 15E3. Evans blue levels in ears sensitized with patient plasma from mice treated with the antibody cocktail were decreased to background Evans blue levels as measured in healthy donor sensitized ears.

### Example 6: Multispecific Constructs Based on Parental Antibodies 2F8, 7G6, 17H9 and 15E3

In view of the efficacy of the cocktail of antibodies 2F8, 7G6, 17H9 and 15E3, in a next step different multispecific constructs including the binding sites (variable regions) of parental antibodies 2F8, 7G6, 17H9 and 15E3 were designed and prepared.

Briefly, two bispecific constructs including either (i) the binding sites of 15E3 and 2F8; or (ii) the binding sites of 17H9 and 7G6 were designed and prepared. To this end, bispecific IgG (H)-scFv fusion constructs as described in Coloma, M., Morrison, S. Design and production of novel tetravalent bispecific antibodies. Nat Biotechnol 15, 159-163 (1997). https://doi.org/10.1038/nbt0297-159, with the VH/VL sequences of either (i) 15E3 (as Fab domains) and 2F8 (as heavy chain C-terminal scFv fusion); or (ii) 17H9 (as Fab domains) and 7G6 (as heavy chain C-terminal scFv fusion) were designed and prepared. For each bispecific antibody, two constructs were prepared using either IgG1 (SEQ ID NO: 59) or IgG4 S228P (SEQ ID NO: 60) as IgG.

For trispecific constructs, binding sites of 17H9, 15E3 and 7G6 were combined. To this end, a heterodimer of IgG (H)-scFvs was constructed with same Fab domains of 17H9 (and scFv of 7G6 and 15E3) using knob-into-hole CH3 as described in Ridgway J B, Presta L G, Carter P. Knobs-into-holes' engineering of antibody CH3 domains for heavy chain heterodimerization. Protein Eng. 1996 July; 9 (7): 617-21. doi: 10.1093/protein/9.7.617, wherein the Fab was the same for both antibody halves (17H9) and the respective scFvs were 15E3 and 7G6. IgG1 was used as IgG. The resulting heterodimer was isolated by Protein A purification followed by mixed-mode chromatography according to Tang et al., 2020 (Jiaqin Tang, Xudong Zhang, Tao Chen, Ying Wang, Yifeng Li, Removal of half antibody, hole-hole homodimer and aggregates during bispecific antibody purification using MMC ImpRes mixed-mode chromatography, Protein Expression and Purification, Volume 167, 2020, 105529, ISSN 1046-5928, https://doi.org/10.1016/j.pep.2019.105529).

In a similar manner, a tetraspecific antibody was constructed with binding sites of each of 2F8, 7G6, 17H9 and 15E3. Specifically, a heterodimer of IgG (H)-scFv and scFv-Fc-scFv (IgG-scFv/scFv-Fc-scFv IgG1 heterodimer) was designed using knob-into-hole CH3 as described in Ridgway J B, Presta L G, Carter P. ‘Knobs-into-holes’ engineering of antibody CH3 domains for heavy chain heterodimerization. Protein Eng. 1996 July; 9 (7): 617-21. doi: 10.1093/protein/9.7.617. Pure heterodimer was isolated by Protein A followed by sequential Protein L and CH1 affinity purification.

A further tetraspecific construct (IgG-scFv/scFv-Fc-scFv IgG4 S228P heterodimer) with binding sites of each of 2F8, 7G6, 17H9 and 15E3 was designed as IgG4 S228P (SEQ ID NO: 60) heavy chain heterodimerization as described, for example, in: Sampei Z, Igawa T, Soeda T, Okuyama-Nishida Y, Moriyama C, Wakabayashi T, et al. (2013) Identification and Multidimensional Optimization of an Asymmetric Bispecific IgG Antibody Mimicking the Function of Factor VIII Cofactor Activity. PLOS ONE 8 (2): e57479; or in: Kitazawa, T., Igawa, T., Sampei, Z. et al. A bispecific antibody to factors IXa and X restores factor VIII hemostatic activity in a hemophilia A model. Med Nat 18, 1570-1574 (2012). https://doi.org/10.1038/nm.2942). Purification for this IgG4-tetraspecific construct was done as described for the IgG1 tetra-specific construct above.

### Example 7: Multispecific Constructs Based on Parental Antibodies 2F8, 7G6, 17H9 and 15E3 Inhibit Patient's IgE Binding to Peanut Allergens

The cocktail of antibodies 2F8, 7G6, 17H9 and 15E3 and the multispecific constructs based on parental antibodies 2F8, 7G6, 17H9 and 15E3, as described in Example 6 above, were then tested for their efficacy in inhibiting patient IgE binding to peanut extract.

Briefly, ELISA plates were coated with anti-IgE (10 ug/ml), overnight at 4° C. and IgE from peanut allergic patients (n=16) were captured (2 h incubation at room temperature). Biotinylated peanut extract (1.12 nM) was pre-incubated with the antibody cocktail (“MY006-4 cocktail”) or different multispecific antibodies (80 nM) for 1 h at room temperature. Allergen binding to IgE in presence of antibodies was measured using anti-strep HRP as read-out. % IgE binding inhibition was calculated as: % inhibition=100−(OD450_{Ab}/OD450_{w/o Ab}*100) and normalized to assay internal control.

The following multispecific antibodies were used:
“2× MY006 bi-specific”: includes the two bispecific constructs using IgG1 as described in Example 6 (IgG (H)-scFv format).
“MY006-trispecific”: the IgG (H)-scFv heterodimer as described in Example 6 was used.
“MY006-tetraspecific”: the IgG-scFv/scFv-Fc-scFv IgG1 heterodimer combining the binding sites of all four antibodies 2F8, 7G6, 17H9 and 15E3 as described in Example 6.

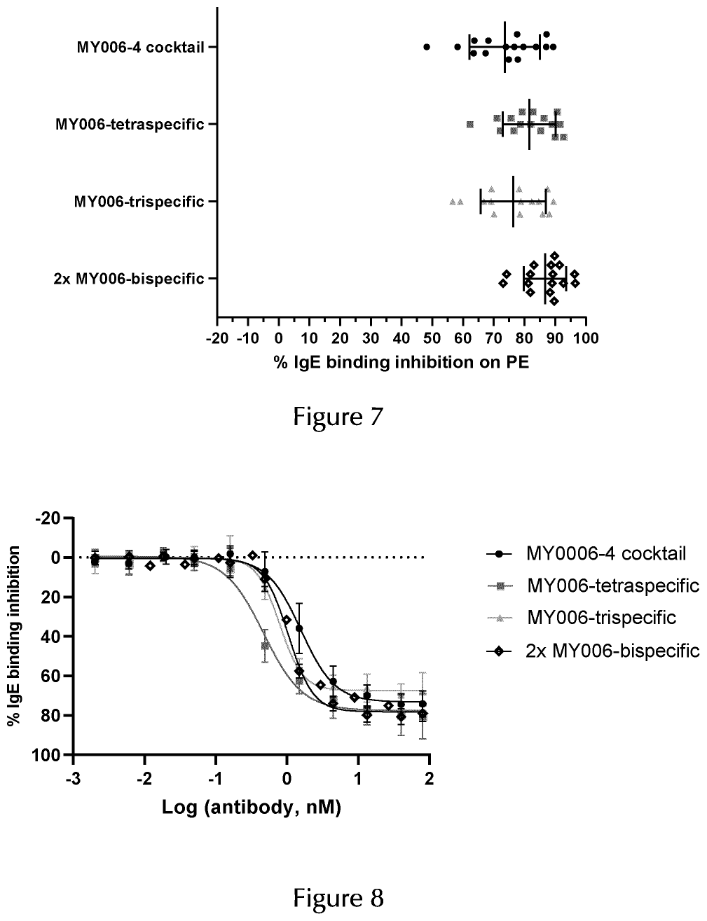

Results are shown in FIG. 7. The data show that the cocktail of antibodies 2F8, 7G6, 17H9 and 15E3 as well as the different multispecific constructs based on parental antibodies 2F8, 7G6, 17H9 and 15E3 inhibit patient's IgE binding to peanut allergens.

### Example 8: Multispecific Constructs Based on Parental Antibodies 2F8, 7G6, 17H9 and 15E3 Inhibit Patient's IgE Binding to Peanut Allergens

The cocktail of antibodies 2F8, 7G6, 17H9 and 15E3 and the multispecific constructs based on parental antibodies 2F8, 7G6, 17H9 and 15E3 were then tested for their efficacy in inhibiting binding of IgEs derived from a pool containing plasma from 90 peanut allergic patients to peanut extract.

Briefly, ELISA plates were coated with anti-IgE (10 ug/ml), overnight at 4° C. and IgE from peanut allergic patients were captured (2 h incubation at room temperature). Biotinylated peanut extract (1.12 nM) was pre-incubated with the antibody cocktail (“MY006-4 cocktail”) or the different multispecific antibodies (“2× MY006 bi-specific”, “MY006-trispecific” or “MY006-tetraspecific”) as described in Example 7 above (80 nM, titrated) for 1 h at room temperature. Allergen binding to IgE in presence of antibodies was measured. % IgE binding inhibition was calculated as: % inhibition=100-(OD450Ab/OD450w/o Ab*100) and normalized to assay internal control.

Results are shown in FIG. 8. The following IC50 values were obtained:
MY006-4:1571 pM
MY006-tetraspecific: 451 PM
MY006-trispecific: 788 pM
2× MY006-bispecific: 1047 pM

These data confirm that the cocktail of antibodies 2F8, 7G6, 17H9 and 15E3 as well as the different multispecific constructs based on parental antibodies 2F8, 7G6, 17H9 and 15E3 inhibit patients' IgE binding to peanut allergens.

### Example 9: Multispecific Constructs Based on Parental Antibodies 2F8, 7G6, 17H9 and 15E3 Inhibit Basophil Degranulation

The cocktail of antibodies 2F8, 7G6, 17H9 and 15E3 and the multispecific constructs based on parental antibodies 2F8, 7G6, 17H9 and 15E3, as described in Example 6 above, were then tested for their efficacy in inhibiting basophil degranulation.

Briefly, Leukocytes from healthy blood donors were isolated and surface IgE was removed by incubation with lactic acid. Leukocytes were re-sensitized with IgE by incubation with a pool containing plasma from 90 peanut allergic patients. Re-sensitized leukocytes were stimulated with peanut extract (PE, 0.5 nM) in the presence of the antibody cocktail (“MY006-4 cocktail”) or the different multispecific antibodies (“2× MY006 bi-specific” or “MY006-tetraspecific”). Quantification of sLT by ELISA (Bühlmann EK CAST).

The following multispecific antibodies were used:
“2× MY006 bi-specific”: includes the two bispecific constructs using IgG4 S228P as described in Example 6 (IgG (H)-scFv format).
“MY006-tetraspecific”: the IgG-scFv/scFv-Fc-scFv IgG4 S228P heterodimer combining the binding sites of all four antibodies 2F8, 7G6, 17H9 and 15E3 as described in Example 6.

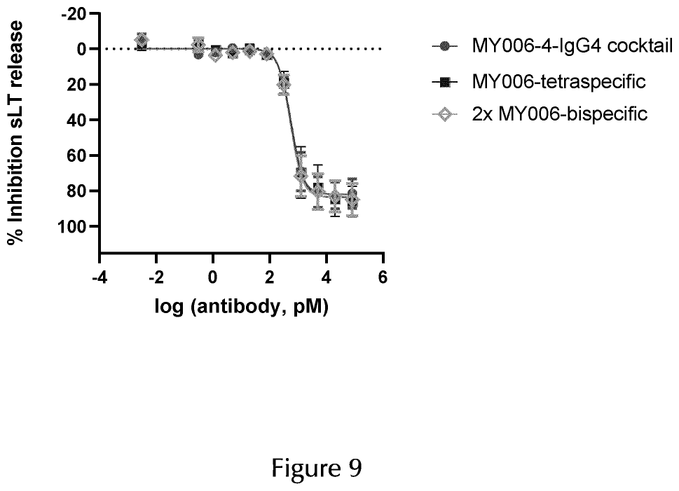

Results are shown in FIG. 9. The following IC50 values were obtained:
MY006-4 cocktail: 536.7 pM
MY006-tetraspecific: 568.7 pM
2× MY006-bispecific: 533.1 pM

In summary, these data indicate that the cocktail of antibodies 2F8, 7G6, 17H9 and 15E3 and the multispecific constructs based on parental antibodies 2F8, 7G6, 17H9 and 15E3 inhibit basophil degranulation (allergen-mediated release of leukotrienes (sLT)) to the same extent.

### Example 10: Multispecific Constructs Based on Parental Antibodies 2F8, 7G6, 17H9 and 15E3 Prevents Anaphylaxis in a Mouse Model of Peanut Allergy

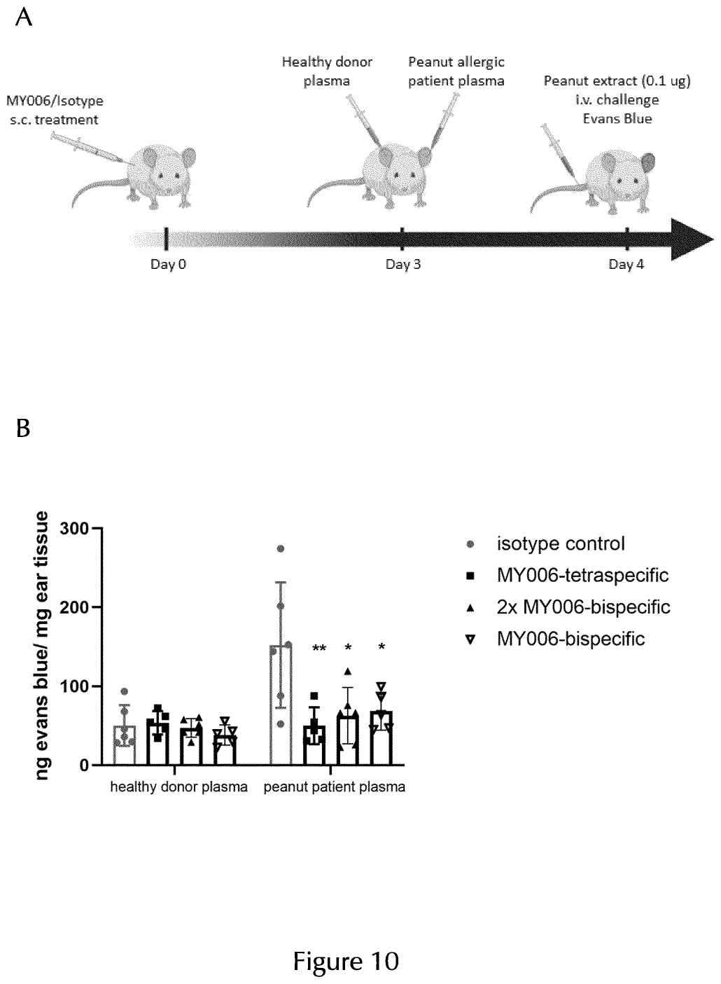

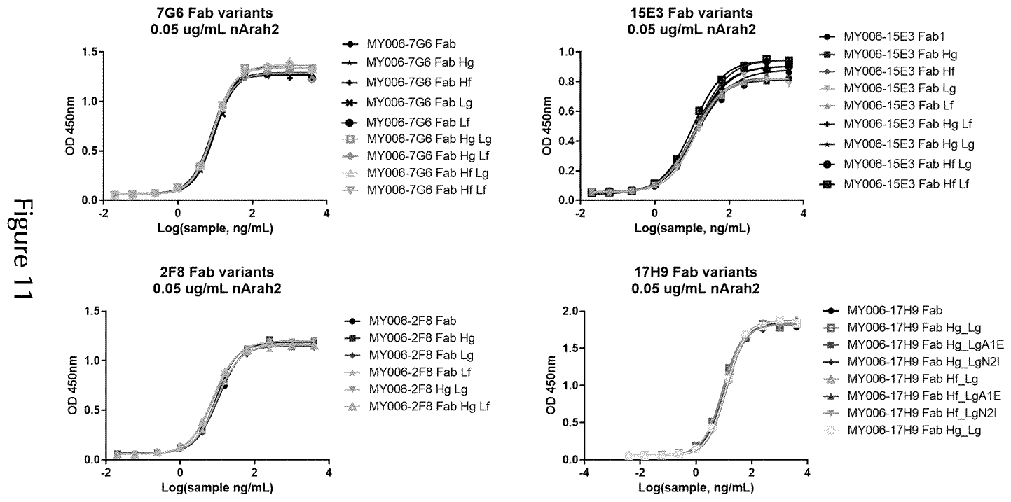

Next, the effects of multispecific constructs combining the specificities of 2F8, 7G6, 17H9 and 15E3 were tested in a humanized mouse model recapitulating anaphylaxis. Briefly, mice with a humanized FcεRI (Genoway, France) were passively sensitized by injection of healthy donor plasma or peanut allergic patient plasma into each ear (passive cutaneous anaphylaxis, PCA). 24 h after sensitization, mice were challenged by intravenous injection of 0.1 ug peanut extract (PE) together with Evans blue (0.5%). Local allergic reaction in the mouse ears resulted in extravasation of the Evans blue staining solution. The amount of extravasated Evans blue was measured by optical absorption at 620 nm and normalized to the weight of the ear tissue collected (Evans blue ng/mg ear tissue). To study the effect of multispecific antibodies, 2.5 mg/kg of each multispecific antibody or an isotype control were injected subcutaneously 4 days prior to PE challenge. The experimental schedule is shown in FIG. 10A.

The following multispecific antibodies were used:
“2× MY006 bi-specific”: includes the two bispecific constructs using IgG4 S228P as described in Example 6 (IgG (H)-scFv format).
“MY006-tetraspecific”: the IgG-scFv/scFv-Fc-scFv IgG4 S228P heterodimer combining the binding sites of all four antibodies 2F8, 7G6, 17H9 and 15E3 as described in Example 6.
“MY006 bi-specific”: only a single bispecific construct using IgG4 S228P was used, namely, the bispecific antibody with the binding sites of 17H9 and 7G6, as described in Example 6 (IgG (H)-scFv format).

The results are shown in FIG. 10B. These data show that all multispecific antibodies, i.e. the tetraspecific, the two bispecific, or the single bispecific antibody alone, significantly reduced the local allergic reaction in the ears of sensitized mice, whereas ears of mice treated with the isotype control had a visible and measurable coloration (mean=152.1 ng/mg). Evans blue levels in ears sensitized with patient plasma from mice treated with the multispecifics were decreased to background Evans blue levels as measured in healthy donor sensitized ears.

### Example 11: Framework Engineering of Antibodies 2F8, 7G6, 17H9 and 15E3

In view of the excellent efficacy of antibodies 2F8, 7G6, 17H9 and 15E3 administered as a cocktail or in a multispecific antibody construct, the framework regions of these antibodies were reverted to the germline sequences (by replacing somatic mutations of the isolated antibodies with the respective amino acid residues according to the germline sequence) in order to reduce the potential risk of immunogenicity of the antibodies.

Two ‘germlined’ version of each variable (V) region were prepared: One version, where all the somatic mutations in the framework regions were reverted (called ‘fullgerm’, ‘f’) and another version, which followed a more conservative approach by avoiding mutations in the Vernier zones (called ‘germ’, ‘g’). Thus, for each variable versions, the wildtype, ‘fullgerm (f)’ and ‘germ (g)’ were designed. The different H and L chains were produced in a combinatorial fashion as shown in Table 6 below:

TABLE 6
Overview over different germlined versions

VH

| VL | wt | germ | full germ |
|---|---|---|---|
| wt | wt | Hg | Hf |
| germ | Lg | Hg_Lg | Hf_Lg |
| full germ | Lf | Hg_Lf | Hf_Lf |

Table 7 below provides an overview over the sequences (SEQ ID NOs) for wild-type (wt) and germlined VH/VL sequences of antibodies 17H9, 15E3, 2F8 and 7G6:

TABLE 7
Overview table for germlined sequences

| | VH | | | | VL | | | |
|---|---|---|---|---|---|---|---|---|
| | | engineered | | | | engineered | | |
| Antibody | wt | Hg | Hf | add. VH | wt | Lg | Lf | add. VL |
| 17H9 | 7 | 44 | 45 | — | 8 | 46 | 47 | 48, 49 |
| 15E3 | 15 | 37 | 38 | — | 16 | 39 | 40 | — |
| 2F8 | 23 | 41 | — | 50 | 24 | 42 | 43 | — |
| 7G6 | 31 | 33 | 34 | — | 32 | 35 | 36 | — |

For antibody 17H9, two additional germlined VL sequences were engineered, namely, 17H9_Lg_A1E and 17H9_Lg_N21 (SEQ ID NOs 48 and 49). For antibody 2F8 an additional VH sequence (2F8_Hg_M115Y; SEQ ID NO: 50) was designed and prepared. Table 8 below provides an overview over the mutations introduced into the different engineered V regions.

The following germlined VH/VL sequences were designed:

TABLE 8
overview over the mutations introduced into the different engineered V regions.

| VH/VL | SEQ ID NO | Remarks |
|---|---|---|
| 7G6_Hg | SEQ ID NO: 33 | reversion of germline residues at S45A, D47G, |
| | | F88Y, D92N, V96A |
| 7G6_Hf | SEQ ID NO: 34 | reversion of germline residues at R44Q, S45A, |
| | | D47G, S82N, F88Y, D92N, V96A |
| 7G6_Lg | SEQ ID NO: 35 | reversion of germline residues at E86D, I101V, |
| | | V126I, N127K |
| 7G6_Lf | SEQ ID NO: 36 | reversion of germline residues at E1D, L4M, E86D, |
| | | I101V, V126I, N127K |
| 15E3_Hg | SEQ ID NO: 37 | reversion of germline residues at A11G, N77T, |
| | | V86S, T93S, S95R, D97E, G100A, I122T |
| 15E3_Hf | SEQ ID NO: 38 | reversion of germline residues at A11G, I42V, |
| | | N77T, S85N, V86S, T93S, S95R, D97E, G100A, |
| | | 1122T |
| 15E3_Lg | SEQ ID NO: 39 | reversion of germline residues at I11V, R47G, |
| | | R51K, H101D |
| 15E3_Lf | SEQ ID NO: 40 | reversion of germline residues at I11V, R47G, |
| | | R51K, F55Y, S85T, H101D, F103Y |
| 2F8_Hg | SEQ ID NO: 41 | reversion of germline residues at K14Q, H88Y, |
| | | A97E, N120K, I125T |
| 2F8_Lg | SEQ ID NO: 42 | reversion of germline residue at D70G |
| 2F8_Lf | SEQ ID NO: 43 | reversion of germline residues at M541, D70G |
| 17H9_Hg | SEQ ID NO: 44 | reversion of germline residues at E5V, Y7S, V24A, |
| | | D92N, N93S, V96A |
| 17H9_Hf | SEQ ID NO: 45 | reversion of germline residues at E5V, Y7S, |
| | | V24A, V78I, D92N, N93S, V96A |
| 17H9_Lg | SEQ ID NO: 46 | reversion of germline residue at R46P |
| 17H9_Lf | SEQ ID NO: 47 | reversion of germline residues at A1E, N2I, R46P |
| 17H9_Lg_A1E | SEQ ID NO: 48 | reversion of germline residues at A1E, R46P |
| 17H9_Lg_N2I | SEQ ID NO: 49 | reversion of germline residues at N2I, R46P |
| 2F8_Hg_M115Y | SEQ ID NO: 50 | reversion of germline residues at K14Q, H88Y, |
| | | A97E, N120K, I125T; removal of potential |
| | | methionine oxidation site: M115Y |

Next, the effect of the engineered variable regions on antigen binding was tested. Briefly, ELISA plates were coated with respective Ara h proteins (Ara h 2:0.05 ug/ml, Ara h 6:0.1 ug/ml, Ara h 3 loop (2nd): 0.1 ug/ml) overnight at 4° C. Afterwards, plates were blocked with BSA (2% in PBS) for 1 hour at room temperature. Fab fragment titration series starting from 4 ug/ml was prepared, added to the single ELISA plate wells and incubated for 2 h at room temperature. The read-out was performed by adding anti-hu-kappa/lambda-HRP (anti-kappa Invitrogen (A18853) and anti-lambda abcam (ab99811) for 1 h at room temperature. Tetramethylbenzidine substrate solution (TMB, Sigma-Aldrich Chemie GmbH, Buchs, Switzerland) was added, then after 5 minutes of hydrolysis time, the reaction was stopped by the addition of 1 M H₂SO₄ and the absorbances were read at 450 nm.

Results are shown in FIG. 11. These data show that reversion to germline residues did not affect the functionality of the antibodies, as they were still able to bind to Ara h 2. In addition, the binding to Ara h 3 or Ara h 6 was also found to be unaffected by the framework engineering unaffected.

In addition, thermal stability of the germlined antibody variants was determined. Thermal stability of biologics is usually directly proportional to overall protein stability. Therefore, the melting points of the germline engineered antibodies (IgG1-Fab format) were measured by differential scanning fluorimetry (DSF). Fabs (5 ug total protein) were mixed with SYPRO Orange dye in Protein Thermal Shift™ Buffer (appliedbiosystems). This dye interacts with the hydrophobic residues usually enriched in the core of proteins which are only exposed by denaturation. Interaction with hydrophobic residues and the dye results in an increase of fluorescence intensity of SYPRO orange. Change of fluorescence signal along a temperature ramp (Heat rate: 0.7° C./min) was measured using a StepOnePlus Real-Time PCR system (appliedbiosystems). Melting curves were analysed and Tm was determined with the Protein Thermal Shift™ Software (Applied Biosystems).

Results are shown in Tables 9-12 below.

TABLE 9
Thermal stability data of the 17H9 germlined variants

| | Tm [° C.] |
|---|---|
| MY006-17H9 Fab | 72.71 |
| MY006-17H9 Fab Hg | 70.53 |
| MY006-17H9 Fab Hf | 70.60 |
| MY006-17H9 Fab Lg | 73.81 |
| MY006-17H9 Fab Lf | 75.84 |
| MY006-17H9 Fab Hg Lg | 71.47 |
| MY006-17H9 Fab Hg Lf | 74.62 |
| MY006-17H9 Fab Hf Lg | 71.40 |
| MY006-17H9 Fab Hf Lf | 74.69 |
| MY006-17H9 Fab Hg_LgA1E | 70.66 |
| MY006-17H9 Fab Hg_LgN21 | 74.75 |
| MY006-17H9 Fab Hf_LgA1E | 70.88 |
| MY006-17H9 Fab Hf_LgN2I | 74.91 |

TABLE 10
Thermal stability data of the 15E3 germlined variants

| | Tm [° C.] |
|---|---|
| MY006-15E3 Fab | 70.30 |
| MY006-15E3 Fab Hg | 75.63 |
| MY006-15E3 Fab Hf | 74.82 |
| MY006-15E3 Fab Lg | 69.79 |
| MY006-15E3 Fab Lf | 72.33 |
| MY006-15E3 Fab Hg Lg | 74.87 |
| MY006-15E3 Fab Hg Lf | 76.53 |
| MY006-15E3 Fab Hf Lg | 74.44 |
| MY006-15E3 Fab Hf Lf | 76.19 |

TABLE 11
Thermal stability data of the 2F8 germlined variants

| | Tm [° C.] |
|---|---|
| MY006-2F8 Fab | 72.68 |
| MY006-2F8 Fab Hg | 74.15 |
| MY006-2F8 Fab Lg | 73.99 |
| MY006-2F8 Fab Lf | 74.64 |
| MY006-2F8 Fab Hg Lg | 75.18 |
| MY006-2F8 Fab Hg Lf | 76.37 |

TABLE 12
Thermal stability data of the 7G6 germlined variants

| | Tm [° C.] |
|---|---|
| MY006-7G6 Fab | 83.07 |
| MY006-7G6 Fab Hg | 84.66 |
| MY006-7G6 Fab Hf | 87.06 |
| MY006-7G6 Fab Lg | 83.50 |
| MY006-7G6 Fab Lf | 82.29 |
| MY006-7G6 Fab Hg Lg | 84.81 |
| MY006-7G6 Fab Hg Lf | 84.03 |
| MY006-7G6 Fab Hf Lg | 87.17 |
| MY006-7G6 Fab Hf Lf | 86.60 |

These data show that, in general, there is a strong trend towards higher melting points in germline reversed constructs. This suggests that these antibodies are also more stable than their respective wild-type counterparts.

In summary, germline reversion did not negatively affect binding to Ara h 2 and Ara h 3, Ara h 6 respectively, but increased in thermal stability. In addition, germline reversion advantageously also removed glycosylation sites, which were found to be otherwise glycosylated in these antibodies (data not shown).

In view of the binding/stability data, the following engineered variants were found to be particularly advantageous:
15E3: Hf/Lg (SEQ ID NOs 38 and 39, respectively)
2F8: Hg/Lf (SEQ ID NOs 41 and 43, respectively)
7G6: Hf/Lg (SEQ ID NOs 34 and 35, respectively)
17H9: Hf/Lg_N21 (SEQ ID NOs 45 and 49, respectively)

### Example 12: CDR Engineering of Antibody 2F8

Next, product charge homogeneity of the antibodies 15E3, 2F8, 7G6 and 17H9 was assessed. To this end, an ion exchange (IEX) analysis was performed. IEX-HPLC measurement was performed on a Thermo Fisher Vanquish Core HPLC System equipped with a quaternary pump, an autosampler and a UV-multiple wavelength detector. The MabPac SCX-10 cation exchange column (10 μm, 4×250 mm) was purchased from Thermo Fisher. To separate the antibody charge variants, the MabPac SCX-10 column was operated at 1 ml/min at 30° C. with a pH-gradient (CX-1 pH Gradient Buffer A (pH 5.6) and B (pH 10.2) from Thermo Fisher). A linear gradient from 0-100% B in 30 min was used. The absorbance at 280 nm was detected. Injection volume was set to 12.5 L for all antibodies. Prior to the injection, each antibody was diluted with PBS to a concentration of 2 mg/ml and buffer exchanged to CX-1 pH gradient buffer A.

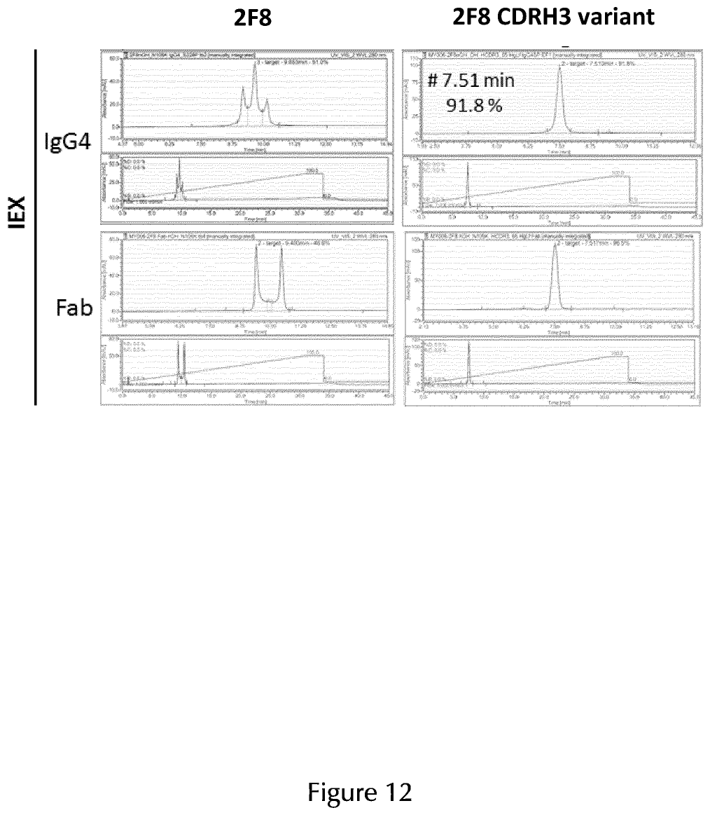

In contrast to the other antibodies, antibody 2F8 showed more than a single peak in the ion exchange analysis (see FIG. 12), indicating that a respective product cannot be obtained in a homogenous manner. In general, for manufacturing of antibodies, a single peak in IEX analysis (indicating a homogenous product) is desirable.

In view thereof, mutations were introduced into the CDRH3 of antibody 2F8 in order to obtain a variant, which can be produced in a homogenous manner. Indeed, the 2F8 variant CDRH3 (SEQ ID NO: 51; VH: SEQ ID NO: 53), obtained by introducing three mutations into the wild-type CDRH3 sequence, resulted in a single peak only, indicating a favorable producibility of such a variant of 2F8 including SEQ ID NO: 51 instead of SEQ ID NO: 19 as CDRH3, as shown in FIG. 12.

Next, the effects of these mutations in the CDRH3 on binding of the 2F8 antibody to both antigens, Ara h 2 and Ara h 6, was tested. Briefly, ELISA plates were coated with respective Ara h proteins (Arah2: 0.05 ug/ml, Arah6: 0.1 ug/ml) overnight at 4° C. Afterwards, plates were blocked with BSA (2% in PBS) for 1 hour at room temperature. Antibody titration series starting from 3 ug/ml was prepared, added to the single ELISA plate wells and incubated for 2 h at room temperature. The read-out was performed by adding anti-hu-IgG-HRP (Jackson ImmunoResearch, West Grove, PA, USA) for 1 h at room temperature. Tetramethylbenzidine substrate solution (TMB, Sigma-Aldrich Chemie GmbH, Buchs, Switzerland) was added, then after 5 minutes of hydrolysis time, the reaction was stopped by the addition of 1 M H₂SO₄ and the absorbances were read at 450 nm.

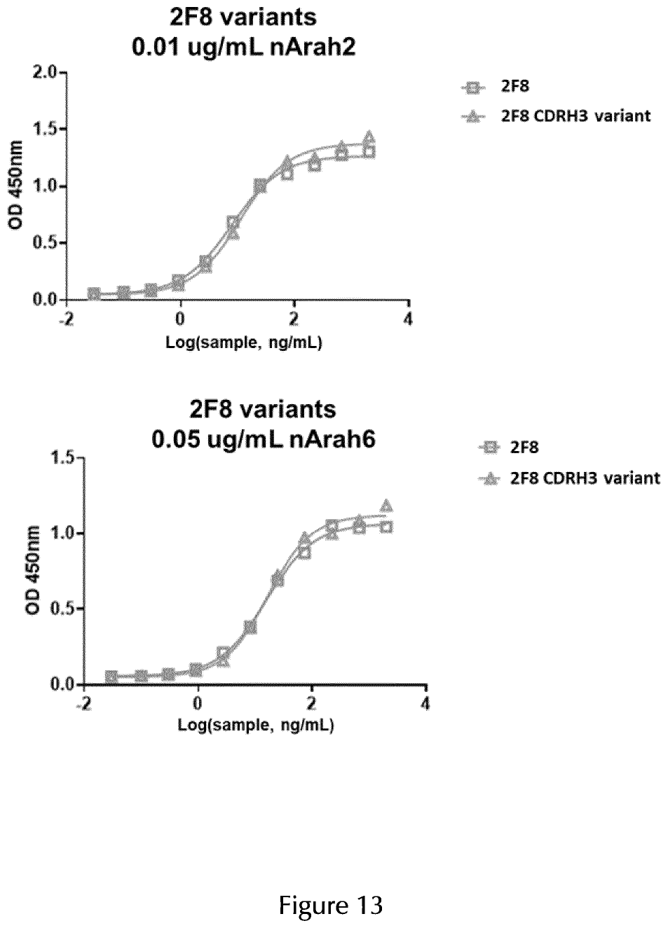

Results are shown in FIG. 13. These data demonstrate that no differences in binding could be observed between 2F8 with the wild-type CDRH3 (SEQ ID NO: 19) or with the CDRH3 variant (SEQ ID NO: 51). In view thereof, 2F8 with the CDRH3 variant (SEQ ID NO: 51) is advantageous, because it can be obtained as homogenous product.

### Example 13: CDR Engineering of Antibody 7G6

In addition, for developability and in vivo pharmacokinetics, non-specific interaction of antibodies to negatively charged molecules should be avoided. Therefore, an insulin/DNA-binding ELISA was used to assess non-specific interactions of the antibodies 15E3, 2F8, 7G6 and 17H9. Briefly, ELISA plates were coated with ssDNA, dsDNA (10 ug/ml) and human insulin (5 ug/ml) overnight at 4° C. Afterwards, plates were blocked with BSA (2% in PBS) for 1 hour at room temperature. Then, four single wells of the ELISA plate were filled with four replicates of the same antibody concentration (10 ug/ml) prepared in dilution buffer and one well was filled with dilution buffer only as control. The read-out was performed by adding anti-hu-IgG-HRP (Jackson ImmunoResearch, West Grove, PA, USA) for 1 h at room temperature. Tetramethylbenzidine substrate solution (TMB, Sigma-Aldrich Chemie GmbH, Buchs, Switzerland) was added and after 5 minutes the reaction was stopped by the addition of 1 M H₂SO₄ and the absorbances were read at 450 nm. Assay scores were calculated as the signal ratio of the antibody at 10 μg/ml binding to DNA or insulin relative to the ELISA signal in the absence of a primary antibody (control well). Non-specific binding to at least two different types of antigens (DNA or Insulin) with an assay score above 10 in at least two independent experiments can be considered unfavorable for antibody development.

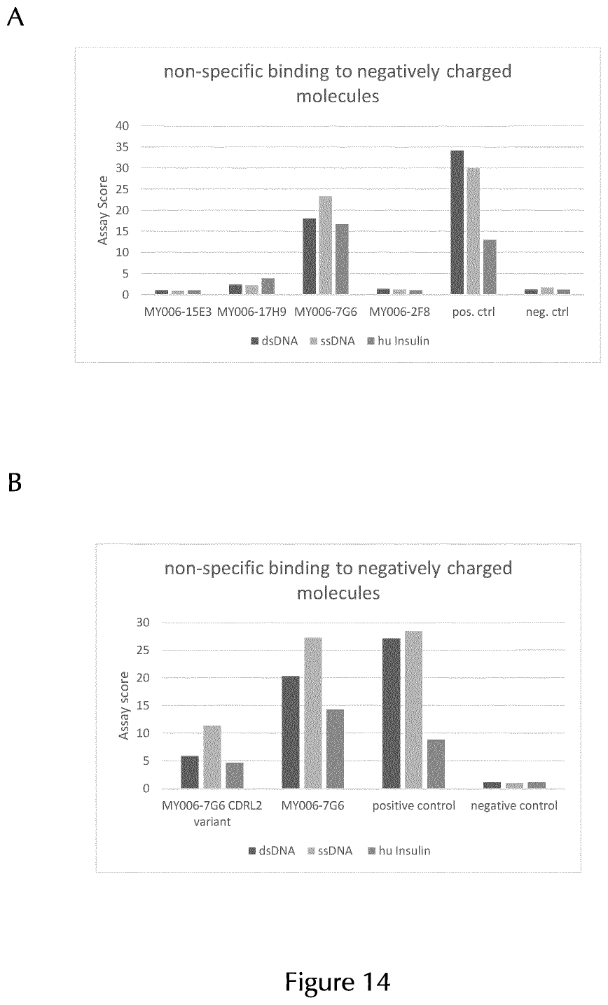

Results are shown in FIG. 14A. These data indicate non-specific interactions for 7G6, but not for the other antibodies.

In view thereof, mutations were introduced into the CDRL2 of antibody 7G6 in order to remove a positive patch. Indeed, non-specific binding was drastically reduced for the 7G6 variant CDRL2 (SEQ ID NO: 52; VL: SEQ ID NO: 54), obtained by introducing a mutation into the wild-type CDRL2 sequence, indicating a favorable developability of such a variant of 7G6 including SEQ ID NO: 52 instead of SEQ ID NO: 29 as CDRL2, as shown in FIG. 14B.

Next, the effects of these mutations in the CDRL2 on binding of the 7G6 antibody to peanut extract (PE) was tested. Briefly, ELISA plates were coated with 0.05 ug/ml PE overnight at 4° C. Afterwards, plates were blocked with BSA (2% in PBS) for 1 hour at room temperature.

Antibody titration series starting from 3 ug/ml was prepared, added to the single ELISA plate wells and incubated for 2 h at room temperature. The read-out was performed by adding anti-hu-IgG-HRP (Jackson ImmunoResearch, West Grove, PA, USA) for 1 h at room temperature. Tetramethylbenzidine substrate solution (TMB, Sigma-Aldrich Chemie GmbH, Buchs, Switzerland) was added, then after 5 minutes of hydrolysis time, the reaction was stopped by the addition of 1 M H₂SO₄ and the absorbances were read at 450 nm.

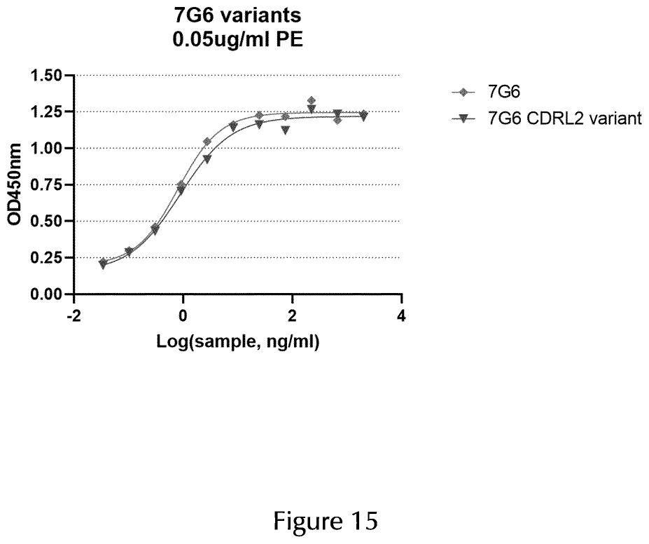

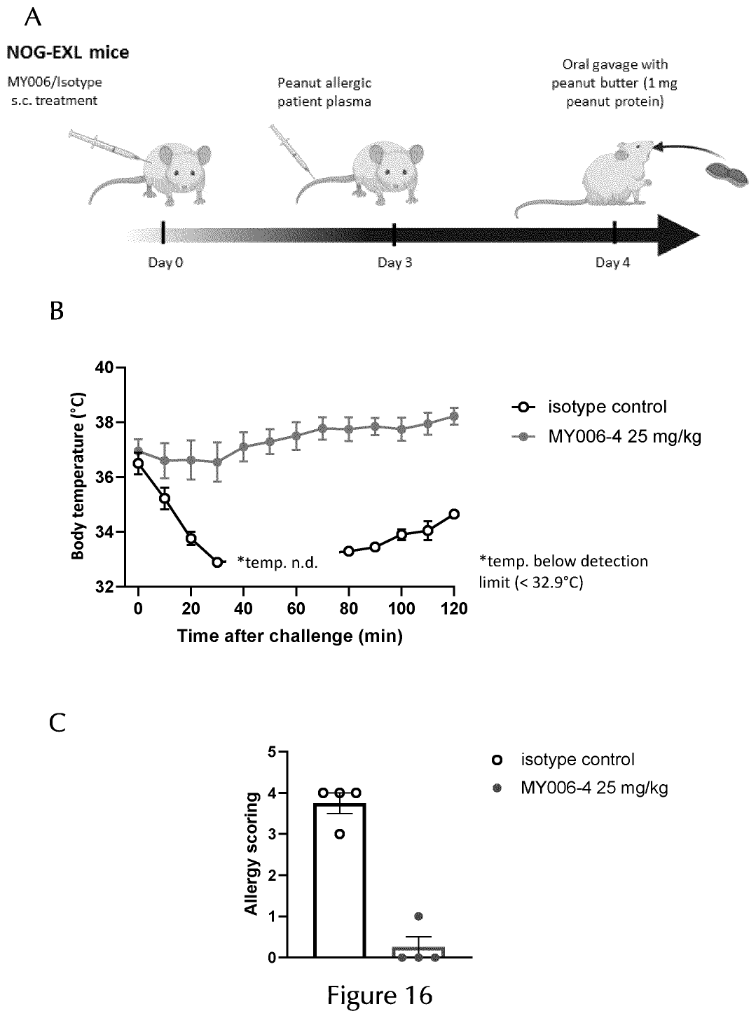

Results are shown in FIG. 15. These data demonstrate that no differences in binding could be observed between 7G6 and the 7G6 with the CDRL2 variant (SEQ ID NO: 52). In view thereof, 7G6 with the CDRL2 variant (SEQ ID NO: 52) is advantageous, because non-specific interactions are considerably reduced.

### Example 14: Cocktail of Antibodies 2F8, 7G6, 17H9 and 15E3 Prevents Anaphylaxis in a Humanized Mouse Model of Peanut Allergy

Next, a cocktail of antibodies 2F8, 7G6, 17H9 and 15E3 was tested in a humanized mouse model (NOG-EXL mice). NOG-EXL (NOD.Cg-Prkdc^{scid} Il2^{tg1Sug} Tg (SV40/HTLV-IL3,CSF2) 10-7Jic/JicTac) is an immunodeficient mouse, expressing human GM-CSF and IL-3 cytokines (hGM-CSF/hIL-3 NOG), supporting the differentiation of human myeloid cell lineages. Mice are humanized by engraftment with hematopoietic stem cells (CD34+ from cord blood donors). Engrafted NOG-EXL mice were passively sensitized with plasma from peanut allergic patients (200 μl, i.v.), which resulted in anaphylaxis upon oral gavage of peanut butter (data not shown).

To test efficacy of antibodies, NOG-EXL mice were administered subcutaneously with a cocktail of engineered antibodies 2F8, 7G6 17H9 and 15E3 or isotype control. Three days later, mice were sensitized intravenously (i.v.) with 200 ul of plasma derived from peanut allergic patients. 24 hours after i.v. sensitization, mice were challenged with peanut butter by oral gavage (1 mg peanut protein). The schedule is shown in FIG. 16A.

Body temperature and signs of allergic reactions were measured over 120 minutes upon gavage. Allergy scoring was performed as follows: 0 for no symptoms; 1 for scratching and rubbing around the nose and head; 2 for puffiness around the eyes and mouth, diarrhoea, pilar erecti, reduced activity and/or decreased activity with increased respiratory rate; 3 for wheezing, laboured respiration and cyanosis around the mouth and the tail; 4 for no activity after prodding or tremor and convulsion; 5 for death.

Results are shown in FIG. 16. FIG. 16B shows a pronounced decrease in body temperature of sensitized mice treated with isotype control in comparison to mice treated with the antibody cocktail, indicating an anaphylactic reaction of the control mice, but not in mice treated with the antibody cocktail. FIG. 16C shows the results of allergy scoring, with pronounced scoring for control-treated mice, while no allergic reaction was observed in mice treated with the antibody cocktail. In summary, these data demonstrate that a cocktail of antibodies 2F8, 7G6, 17H9 and 15E3 prevents anaphylaxis in a humanized mouse model of peanut allergy.

### Example 15: Cocktail of Antibodies 2F8, 7G6, 17H9 and 15E3 is Advantageous Over Prior Art Antibodies of WO 2018/234383

Next, the combination of antibodies 2F8, 7G6, 17H9 and 15E3, as described herein, was compared to the antibodies disclosed in WO 2018/234383.

Firstly, a cross-competition ELISA was performed to determine the complementation groups (epitope binning) of antibodies of WO 2018/234383 and as described herein on natural Ara h 2 (nAra h 2). Briefly, ELISA plates were coated with the antibody (1.33 nM; 7G6, 12G10, 37D5, 17H9, 32B10, 2F8, 4B2 or 15E3) overnight at 4° C. Afterwards, plates were blocked with BSA (2% in PBS) for 1 hour at room temperature. At the same time, the biotinylated allergen (nAra h 2=1.2 nM) was pre-incubated with the competitor antibody (as shown in Table 13 below; dilution series with a starting concentration of 80 nM) for at least 1 hour at room temperature. The antibody-allergen mixture was then transferred to the coated ELISA plate and incubated for 2 h at room temperature. The read-out was performed by adding streptavidin-HRP (Biolegend) for 1 h at room temperature. Tetramethylbenzidine substrate solution (TMB, Sigma-Aldrich Chemie GmbH, Buchs, Switzerland) was added and after 5 minutes the reaction was stopped by the addition of 1 M H₂SO₄ and the absorbances were read at 450 nm. Results are shown as % of inhibition calculated as: % inhibition=100-(OD450Ab/OD450w/o Ab*100). Competition between the coated MY-antibody and itself was used as a control for the assay and it defined the maximal percentage of inhibition. The values of % inhibition were then normalized to that internal control. Antibodies were considered as “competing” if more values of more than 80% were obtained, and as “non-competing” for values of less than 20% (values in between would be considered as “partially competing”, but were not obtained here).

Results are shown in Table 13 below:

TABLE 13
Cross-competition ELISA indicating competing
(X) and non-competing (—) antibodies.

| | Group | | | | | | | |
|---|---|---|---|---|---|---|---|---|
| | 1 | 3 | | | 2 | | 4 | |
| Antibodies | 7G6 | 12G10 | 37D5 | 17H9 | 32B10 | 2F8 | 4B2 | 15E3 |
| 7G6 | X | — | — | — | — | — | — | — |
| 12G10 | — | X | X | X | — | — | — | — |
| 37D5 | — | X | X | X | — | — | — | — |
| 17H9 | — | X | X | X | — | — | — | — |
| 32B10 | — | — | — | — | X | X | — | — |
| 2F8 | — | — | — | — | X | X | — | — |
| 4B2 | — | — | — | — | — | — | X | X |
| 15E3 | — | — | — | — | — | — | X | X |

These data show that the antibodies of WO 2018/234383 and the antibodies described herein can be grouped into 4 different epitope groups with regard to Ara h 2, namely:
Group 1: 7G6
Group 2: 32B10, 2F8
Group 3: 12G10, 37D5, 17H9
Group 4: 4B2, 15E3

To compare the binding specificity of the antibodies among the different groups to nAra h 1, nAra h 2, nAra h 3 and nAra h 6, an ELISA was performed. ELISA plates were coated with respective Ara h proteins (Arah1: 0.5 ug/ml, Arah2: 0.01 ug/ml, Arah3: 0.1 ug/ml, Arah6: 0.05 ug/ml) overnight at 4° C. Afterwards, plates were blocked with BSA (2% in PBS) for 1 hour at room temperature. Antibody titration series starting from 3 ug/ml was prepared, added to the single ELISA plate wells and incubated for 2 h at room temperature. The read-out was performed by adding anti-hu-IgG-HRP (Jackson ImmunoResearch, West Grove, PA, USA) for 1 h at room temperature. Tetramethylbenzidine substrate solution (TMB, Sigma-Aldrich Chemie GmbH, Buchs, Switzerland) was added, then after 5 minutes of hydrolysis time, the reaction was stopped by the addition of 1 M H₂SO₄ and the absorbances were read at 450 nm.

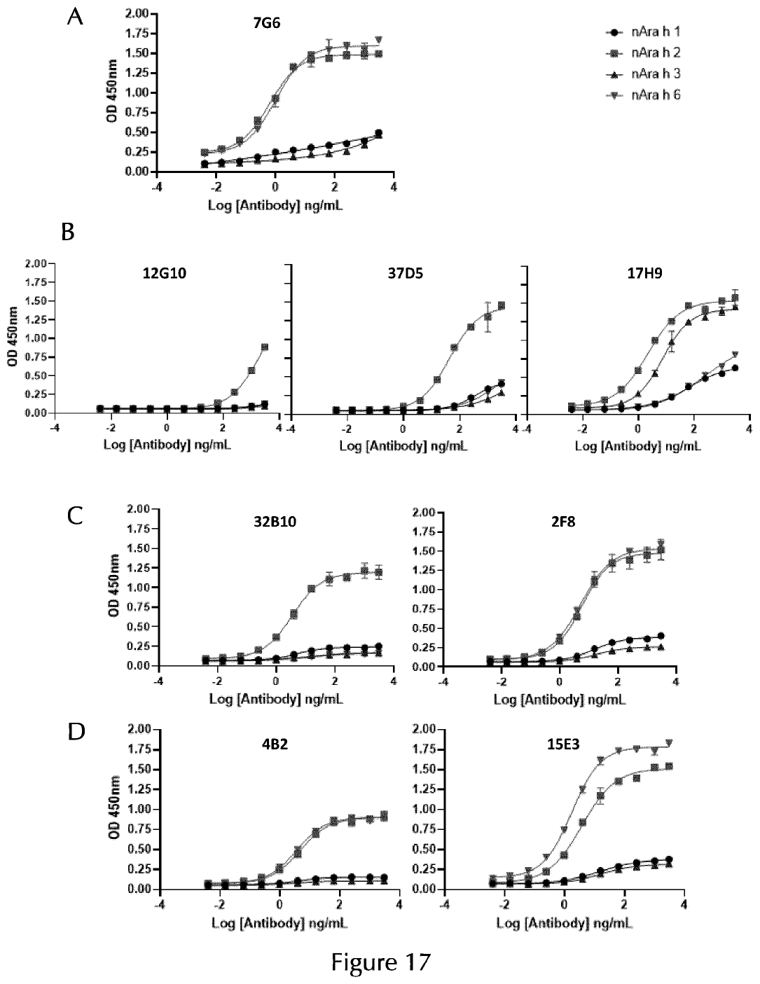

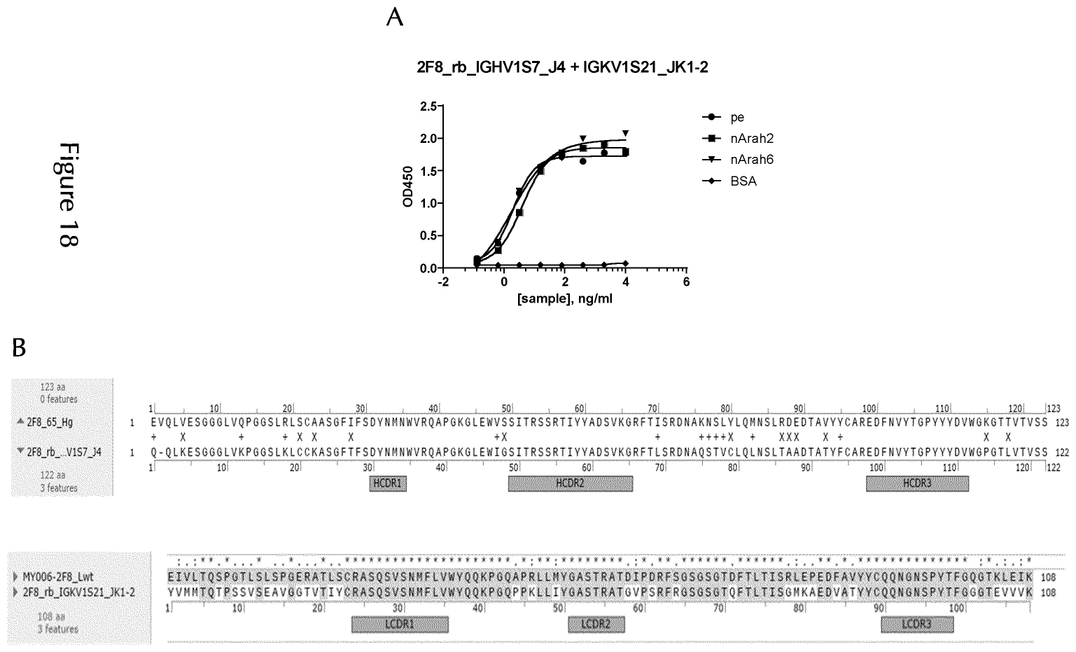

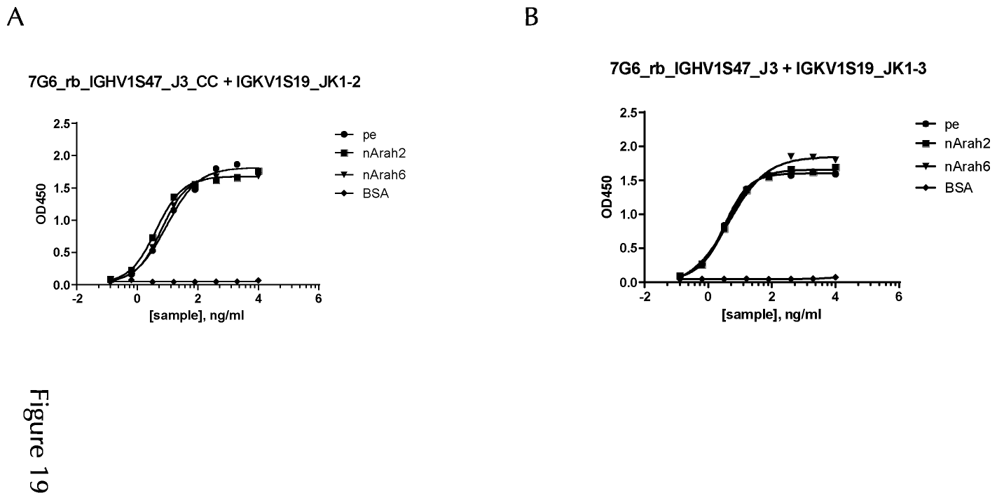

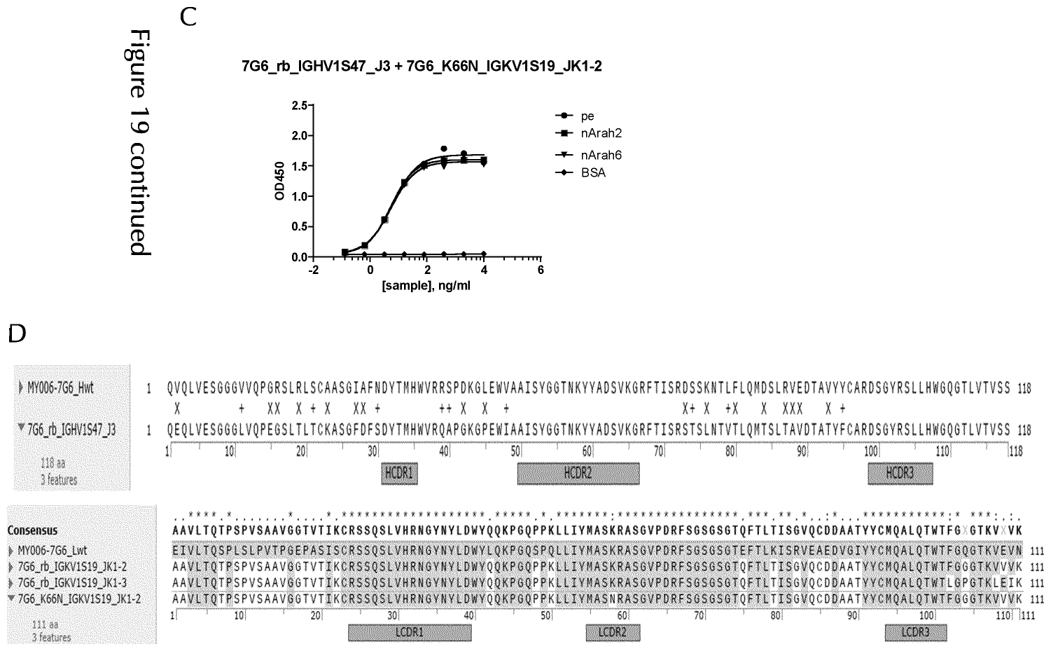

Results are shown in FIG. 17 with Group 1 antibody 7G6 (A), Group 3 antibodies 12G10, 37D5 and 17H9 (B), Group 2 antibodies 32B10 and 2F8 (C), and Group 4 antibodies 4B2 and 15E3 (D). Among the Group 3 antibodies, 12G10 showed the lowest binding affinity to nAra h 2. 17H9 was found to bind specifically to nAra h 2 and nAra h 3, confirming the results of Example 2, while 12G10 and 37D5 bound specifically only to nAra h 2. Among the Group 3 antibodies, 17H9 showed the highest binding affinity to nAra h 2. Among the Group 2 antibodies, 2F8 was found to bind specifically to nAra h 2 and nAra h 6, confirming the results of Example 2, while 32B10 bound specifically only to nAra h 2. While both Group 4 antibodies were found to bind specifically to nAra h 2 and nAra h 6, 15E3 showed considerably higher binding affinity to nAra h 6 as compared to 4B2.

In summary, these data confirm that antibodies 7G6, 17H9, 2F8 and 15E3 bind to distinct, non-overlapping epitopes on nAra h 2. When compared to antibodies of WO 2018/234383 competing for the same epitope on Ara h 2, each of the antibodies 17H9, 2F8 and 15E3 showed increased breadth of binding specificity and/or binding affinity as compared to antibodies of WO 2018/234383 competing for the same epitope. As no further antibody of WO 2018/234383 was found to compete with 7G6 (Group 1), no comparison is available for this group.

### Example 16: Grafting of CDRs onto Distinct Rabbit Variable Framework Regions

Various CDRs, as defined by the classical Kabat scheme, were grafted onto rabbit variable framework regions. These chimeras were expressed as fully rabbit antibodies (constant domains from rabbit IgG). The CDR sequences of (i) 2F8 were used and upon grafting onto rabbit variable framework regions, designated as construct 2F8_rb_IGHV1S7 J4 (SEQ ID NO: 100 presenting VH and HJ)+IGKV1S21_JK1-2 (SEQ ID NO: 101 presenting VK (kappa) and KJ). The CDR sequences of (ii)-7G6 were used and upon grafting onto rabbit variable framework regions, designated as constructs 7G6_rb_IGHV1S47_J3 (SEQ ID NO: 102 presenting VH and HJ)+IGKV1S19_JK1-2 (SEQ ID NO: 103 presenting VK (kappa) and KJ); 7G6_rb_IGHV1S47_J3 (SEQ ID NO: 102 presenting VH and hj)+IGKV1S19_JK1-3 (SEQ ID NO: 104 presenting VK (kappa) and KJ); and 7G6_rb_IGHVIS47_J3 (SEQ ID NO: 102 presenting VH and HJ)+K66N_IGKV1S19_JK1-2 (SEQ ID NO: 105 presenting VK (kappa) (containing the mutation K66N) and KJ). The CDR sequences of (iii) 17H9 were used and upon grafting onto rabbit variable framework regions, designated as constructs 17H9_rb_IGHV1S7_J3 (SEQ ID NO: 106 presenting VH and HJ)+IGKV1S34_JK1-2 (SEQ ID NO: 107 presenting VK (kappa) and KJ); and 17H9_rb_IGHV1S7_J3 (SEQ ID NO: 106 presenting VH and HJ)+IGKV1S34_JK1-5 (SEQ ID NO: 108 presenting VK (kappa) and KJ). The CDR sequences of (iv) 15E3 were used and upon grafting onto rabbit variable framework regions, designated as construct 15E3_rb_IGHV1S8_J5 (SEQ ID NO: 109 presenting VH and HJ)+IGLV2S2_JL5 (SEQ ID NO: 110 presenting VL (lambda) and LJ).

ELISA assays were carried out with the purified rabbit antibodies to test their binding and target specificity to peanut allergens Ara h 2, Ara h6 and/or Ara h 3.

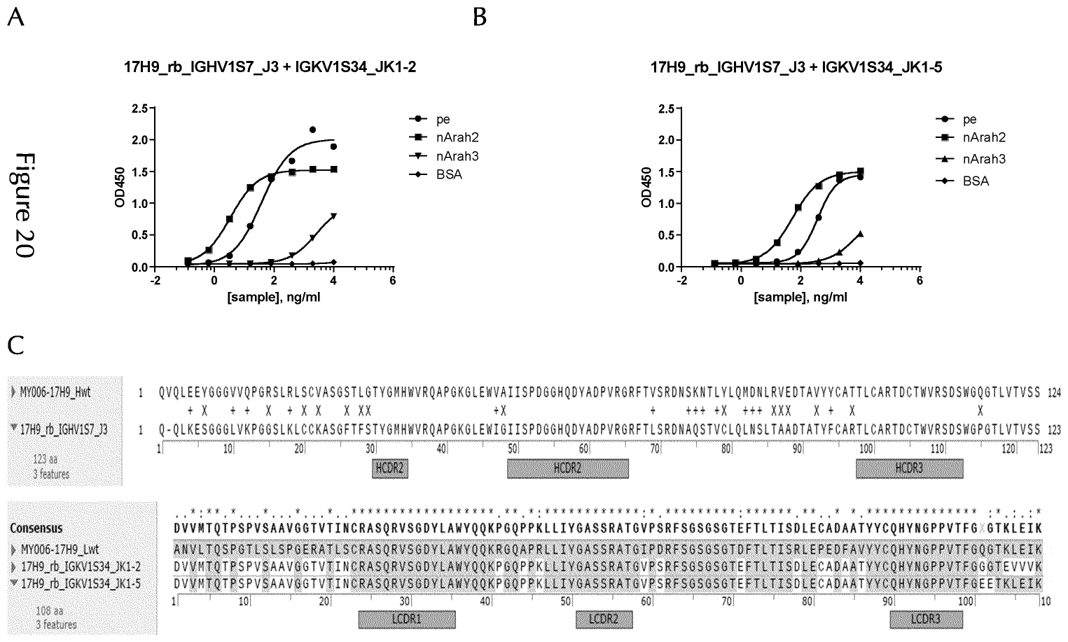

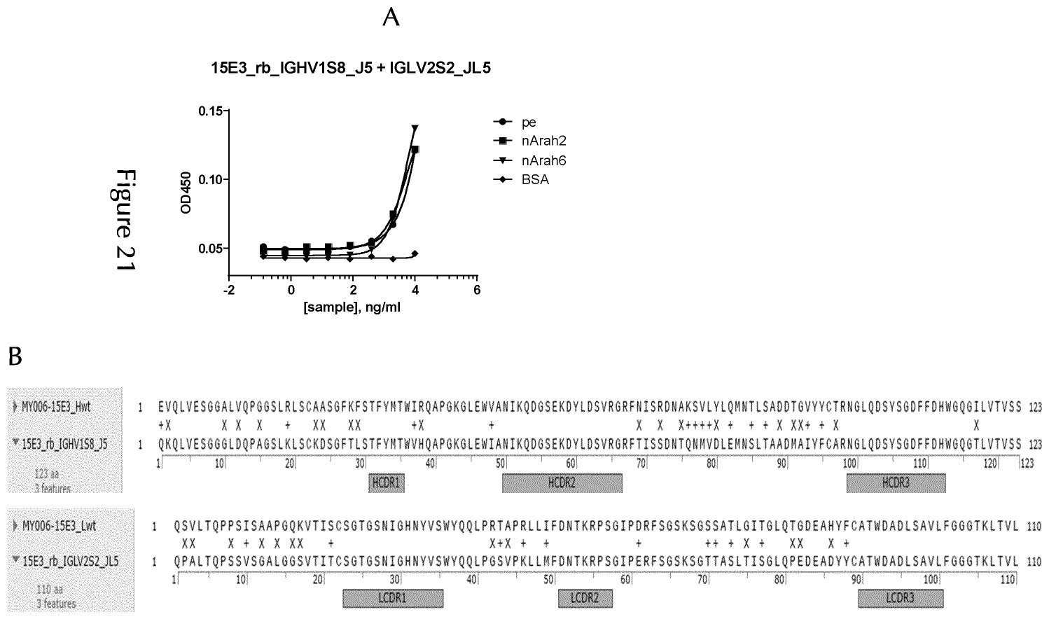

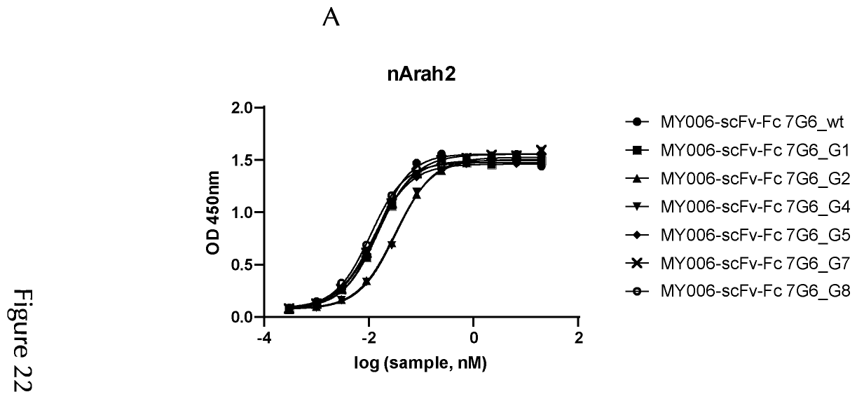

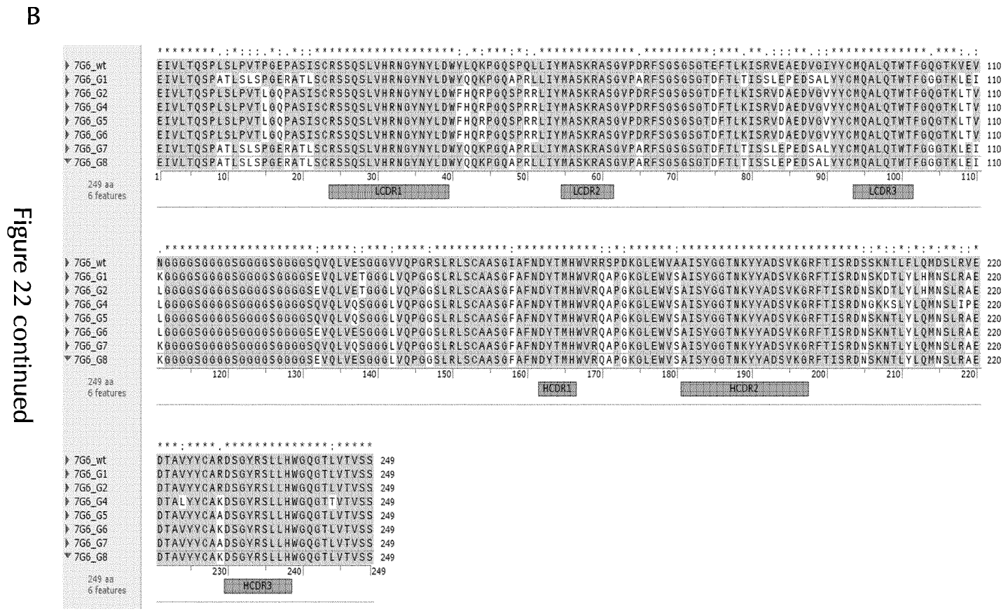

The ELISA data demonstrated that the antibodies containing CDR sequences of2F8, 7G6_{[NP1]}, 17H9 and 15E3 grafted onto rabbit variable regions all retained their original specificity, as shown in FIGS. 18 to 21.

### Example 17: Grafting of CDRs onto Distinct Human Variable Framework Regions

In analogy to Example 16, CDR sequences (7G6 and 15E3) were grafted on various human variable framework region sequences. These constructs were expressed according to the scFv-Fc antibody format. The CDR sequences of 7G6 were used and grafted on various human variable framework regions VK (kappa) and VH encoded by the following genes as presented by Table 14.

TABLE 14

| | VK gene | VH gene |
|---|---|---|
| 7G6_wt | IGKV2-28 | IGHV3-30 |
| 7G6_G1 | IGKV3-11 | IGHV3-23 |
| 7G6_G2 | IGKV2-30 | IGHV3-23 |
| 7G6_G4 | IGKV2-30 | IGHV3-43 |
| 7G6_G5 | IGKV2-30 | IGHV3-43 |
| 7G6_G6 | IGKV2-30 | IGHV3-23 |
| 7G6_G7 | IGKV3-11 | IGHV3-33 |
| 7G6_G8 | IGKV3-11 | IGHV3-23 |

Construct 7G6-G1 is presented as SEQ ID NO: 111, construct 7G6-G2 is presented as SEQ ID NO: 112, 7G6-G4 is presented as SEQ ID NO: 113, 7G6-G5 is presented as SEQ ID NO: 114, 7G6-G6 is presented as SEQ ID NO: 115, 7G6-G7 is presented as SEQ ID NO: 116, and 7G6-G8 is presented as SEQ ID NO: 117.

The CDR sequences of 15E3 were used and grafted on human VK (kappa) variable framework region sequence encoded by gene IGLV1-51 and human VH variable framework region sequence encoded by gene IGHV3-7 (15E3_G4: SEQ ID NO: 118).

TABLE 15

| | VK gene | VH gene |
|---|---|---|
| 15E3_wt | IGLV1-51 | IGHV3-7 |
| 15E3_G4 | IGLV1-51 | IGHV3-7 |

ELISA assays were carried out with the purified antibodies to test their binding and target specificity to peanut allergens Ara h2.

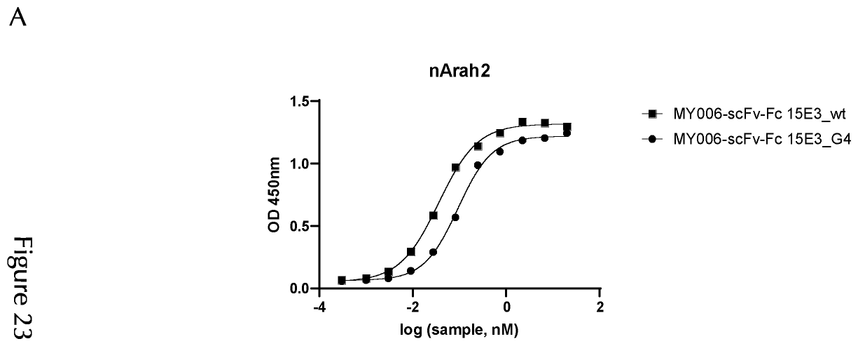

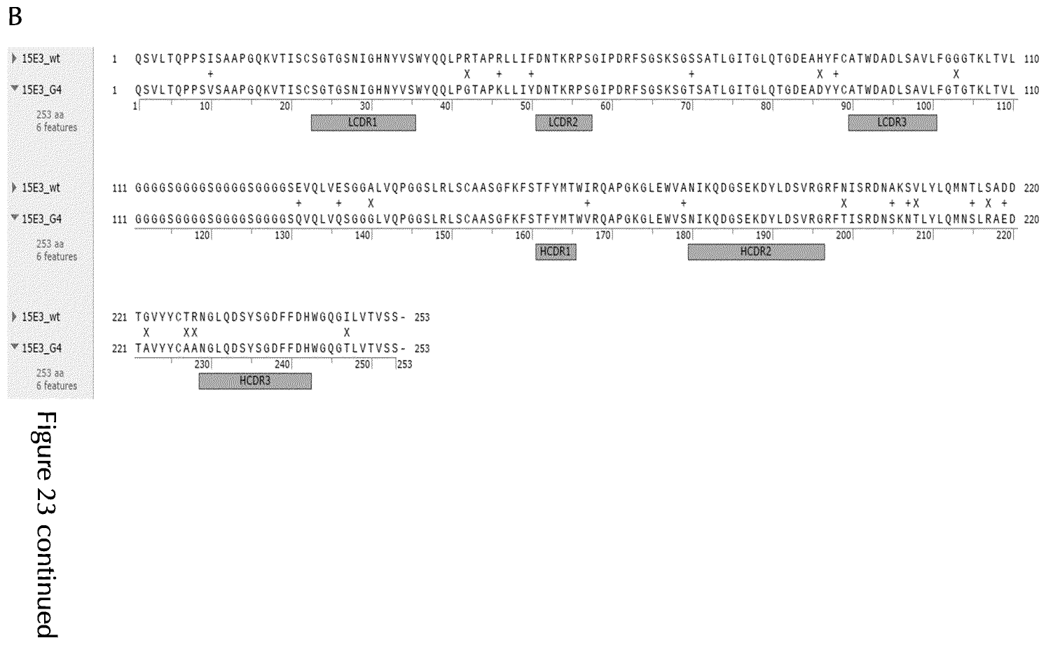

The ELISA data demonstrated that the antibodies containing 7G6 CDRs and 15E3 CDRs, respectively, grafted onto various human variable framework regions all retained their original specificity, as shown in FIGS. 22 and 23.

### Example 18 Immunotherapy Combining Antibody Cocktail and Allergen

Peripheral blood mononuclear cells (PBMCs) were isolated from peanut allergic patients by density gradient centrifugation and cyropreserved. Cryopreserved PBMCs were thawed and stimulated with 1 μg/ml nAra h 2 (peanut allergen) with or w/o “MY006-4 antibody” cocktail (comprising antibodies 2F8, 7G6, 17H9 and 15E3), and incubated at 37° C. After 3 days, supernatant was harvested, and cytokine secretion (IL-10 and IL-13) was assessed by LEGENDPlex (Biolegend).

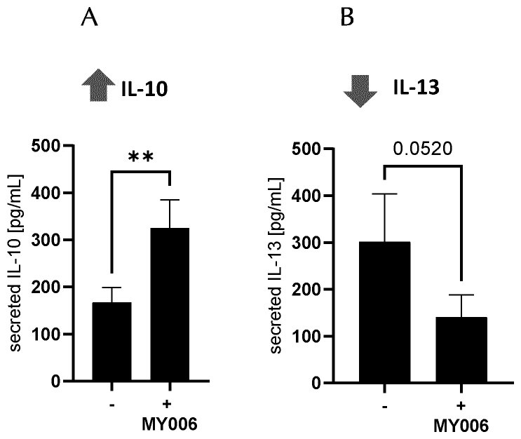

Secretion of the anti-inflammatory cytokine IL-10 was increased upon stimulation by combining the allergen and the “MY006-4” antibody cocktail as compared to stimulation by the allergen alone (FIG. 24A), promoting formation of IgG4 antibodies. Secretion of IL-13 (typically secreted from T cells promoting the allergic response) was decreased by combined stimulation of the allergen and the “MY006-4” antibody cocktail (FIG. 24B).

As a result, the antibody cocktail “MY006-4” according to the invention does not interfere with immunotherapy by an allergen. By the synergistic effects achieved by combining both components, safe desensitization may be achieved by following a simplified protocol. A tolerogenic cytokine milieu is induced by the combined approach.

TABLE OF
SEQUENCES AND SEQ ID NUMBERS (SEQUENCE LISTING):

| | | |
|---|---|---|
| SEQ ID NO | Sequence | Remarks |

Antibody amino acid sequences
17H9

| | | |
|---|---|---|
| SEQ ID NO: 1 | TYGMH | CDRH1 |
| SEQ ID NO: 2 | IISPDGGHQDYADPVRG | CDRH2 |
| SEQ ID NO: 3 | TLCARTDCTWVRSDS | CDRH3 |
| SEQ ID NO: 4 | RASQRVSGDYLA | CDRL1 |
| SEQ ID NO: 5 | GASSRAT | CDRL2 |
| SEQ ID NO: 6 | QHYNGPPVT | CDRL3 |
| SEQ ID NO: 7 | QVQLEEYGGGVVQPGRSLRLSCVASGSTLGTYG | VH |
| | MHWVRQAPGKGLEWVAIISPDGGHQDYADPV | |
| | RGRFTVSRDNSKNTLYLQMDNLRVEDTAVYYC | |
| | ATTLCARTDCTWVRSDSWGQGTLVTVSS | |
| SEQ ID NO: 8 | ANVLTQSPGTLSLSPGERATLSCRASQRVSGDYL | VL |
| | AWYQQKRGQAPRLLIYGASSRATGIPDRFSGSG | |
| | SGTDFTLTISRLEPEDFAVYYCQHYNGPPVTFGQ | |
| | GTKLEIK | |

15E3

| | | |
|---|---|---|
| SEQ ID NO: 9 | TFYMT | CDRH1 |
| SEQ ID NO: 10 | NIKQDGSEKDYLDSVRG | CDRH2 |
| SEQ ID NO: 11 | NGLQDSYSGDFFDH | CDRH3 |
| SEQ ID NO: 12 | SGTGSNIGHNYVS | CDRL1 |
| SEQ ID NO: 13 | DNTKRPS | CDRL2 |
| SEQ ID NO: 14 | ATWDADLSAVL | CDRL3 |
| SEQ ID NO: 15 | EVQLVESGGALVQPGGSLRLSCAASGFKFSTFYM | VH |
| | TWIRQAPGKGLEWVANIKQDGSEKDYLDSVRG | |
| | RFNISRDNAKSVLYLQMNTLSADDTGVYYCTRN | |
| | GLQDSYSGDFFDHWGQGILVTVSS | |
| SEQ ID NO: 16 | QSVLTQPPSISAAPGQKVTISCSGTGSNIGHNYV | VL |
| | SWYQQLPRTAPRLLIFDNTKRPSGIPDRFSGSKS | |
| | GSSATLGITGLQTGDEAHYFCATWDADLSAVLF | |
| | GGGTKLTVL | |

2F8

| | | |
|---|---|---|
| SEQ ID NO: 17 | DYNMN | CDRH1 |
| SEQ ID NO: 18 | SITRSSRTIYYADSVKG | CDRH2 |
| SEQ ID NO: 19 | EDFDVSTGPYYMDV | CDRH3 |
| SEQ ID NO: 20 | RASQSVSNMFLV | CDRL1 |
| SEQ ID NO: 21 | GASTRAT | CDRL2 |
| SEQ ID NO: 22 | QQNGNSPYT | CDRL3 |
| SEQ ID NO: 23 | EVQLVESGGGLVKPGGSLRLSCAASGFIFSDYNM | VH |
| | NWVRQAPGKGLEWVSSITRSSRTIYYADSVKGR | |
| | FTISRDNAKNSLHLQMNSLRDADTAVYYCARED | |
| | FDVSTGPYYMDVWGNGTTVIVSS | |
| SEQ ID NO: 24 | EIVLTQSPGTLSLSPGERATLSCRASQSVSNMFLV | VL |
| | WYQQKPGQAPRLLMYGASTRATDIPDRFSGSG | |
| | SGTDFTLTISRLEPEDFAVYYCQQNGNSPYTFGQ | |
| | GTKLEIK | |

7G6

| | | |
|---|---|---|
| SEQ ID NO: 25 | DYTMH | CDRH1 |
| SEQ ID NO: 26 | AISYGGTNKYYADSVKG | CDRH2 |
| SEQ ID NO: 27 | DSGYRSLLH | CDRH3 |
| SEQ ID NO: 28 | RSSQSLVHRNGYNYLD | CDRL1 |
| SEQ ID NO: 29 | MASKRAS | CDRL2 |
| SEQ ID NO: 30 | MQALQTWT | CDRL3 |
| SEQ ID NO: 31 | QVQLVESGGGVVQPGRSLRLSCAASGIAFNDYT | VH |
| | MHWVRRSPDKGLEWVAAISYGGTNKYYADSVK | |
| | GRFTISRDSSKNTLFLQMDSLRVEDTAVYYCARD | |
| | SGYRSLLHWGQGTLVTVSS | |
| SEQ ID NO: 32 | EIVLTQSPLSLPVTPGEPASISCRSSQSLVHRNGYN | VL |
| | YLDWYLQKPGQSPQLLIYMASKRASGVPDRFSG | |
| | SGSGTEFTLKISRVEAEDVGIYYCMQALQTWTFG | |
| | QGTKVEVN | |

VH/VL with germlined framework regions

| | | |
|---|---|---|
| SEQ ID NO: 33 | QVQLVESGGGVVQPGRSLRLSCAASGIAFNDYT | 7G6_Hg |
| | MHWVRRAPGKGLEWVAAISYGGTNKYYADSV | |
| | KGRFTISRDSSKNTLYLQMNSLRAEDTAVYYCAR | |
| | DSGYRSLLHWGQGTLVTVSS | |
| SEQ ID NO: 34 | QVQLVESGGGVVQPGRSLRLSCAASGIAFNDYT | 7G6_Hf |
| | MHWVRQAPGKGLEWVAAISYGGTNKYYADSV | |
| | KGRFTISRDNSKNTLYLQMNSLRAEDTAVYYCA | |
| | RDSGYRSLLHWGQGTLVTVSS | |
| SEQ ID NO: 35 | EIVLTQSPLSLPVTPGEPASISCRSSQSLVHRNGYN | 7G6_Lg |
| | YLDWYLQKPGQSPQLLIYMASKRASGVPDRFSG | |
| | SGSGTDFTLKISRVEAEDVGVYYCMQALQTWTF | |
| | GQGTKVEIK | |
| SEQ ID NO: 36 | DIVMTQSPLSLPVTPGEPASISCRSSQSLVHRNGY | 7G6_Lf |
| | NYLDWYLQKPGQSPQLLIYMASKRASGVPDRFS | |
| | GSGSGTDFTLKISRVEAEDVGVYYCMQALQTW | |
| | TFGQGTKVEIK | |
| SEQ ID NO: 37 | EVQLVESGGGLVQPGGSLRLSCAASGFKFSTFY | 15E3_Hg |
| | MTWIRQAPGKGLEWVANIKQDGSEKDYLDSVR | |
| | GRFTISRDNAKSSLYLQMNSLRAEDTAVYYCTRN | |
| | GLQDSYSGDFFDHWGQGTLVTVSS | |
| SEQ ID NO: 38 | EVQLVESGGGLVQPGGSLRLSCAASGFKFSTFY | 15E3_Hf |
| | MTWVRQAPGKGLEWVANIKQDGSEKDYLDSV | |
| | RGRFTISRDNAKNSLYLQMNSLRAEDTAVYYCT | |
| | RNGLQDSYSGDFFDHWGQGTLVTVSS | |
| SEQ ID NO: 39 | QSVLTQPPSVSAAPGQKVTISCSGTGSNIGHNY | 15E3_Lg |
| | VSWYQQLPGTAPKLLIFDNTKRPSGIPDRFSGSK | |
| | SGSSATLGITGLQTGDEADYFCATWDADLSAVL | |
| | FGGGTKLTVL | |
| SEQ ID NO: 40 | QSVLTQPPSVSAAPGQKVTISCSGTGSNIGHNY | 15E3_Lf |
| | VSWYQQLPGTAPKLLIYDNTKRPSGIPDRFSGSK | |
| | SGTSATLGITGLQTGDEADYYCATWDADLSAVL | |
| | FGGGTKLTVL | |
| SEQ ID NO: 41 | EVQLVESGGGLVQPGGSLRLSCAASGFIFSDYN | 2F8_Hg |
| | MNWVRQAPGKGLEWVSSITRSSRTIYYADSVKG | |
| | RFTISRDNAKNSLYLQMNSLRDEDTAVYYCARE | |
| | DFDVSTGPYYMDVWGKGTTVTVSS | |
| SEQ ID NO: 42 | EIVLTQSPGTLSLSPGERATLSCRASQSVSNMFLV | 2F8_Lg |
| | WYQQKPGQAPRLLMYGASTRATGIPDRFSGSG | |
| | SGTDFTLTISRLEPEDFAVYYCQQNGNSPYTFGQ | |
| | GTKLEIK | |
| SEQ ID NO: 43 | EIVLTQSPGTLSLSPGERATLSCRASQSVSNMFLV | 2F8_Lf |
| | WYQQKPGQAPRLLIYGASTRATGIPDRFSGSGS | |
| | GTDFTLTISRLEPEDFAVYYCQQNGNSPYTFGQ | |
| | GTKLEIK | |
| SEQ ID NO: 44 | QVQLVESGGGVVQPGRSLRLSCAASGSTLGTYG | 17H9_Hg |
| | MHWVRQAPGKGLEWVAIISPDGGHQDYADPV | |
| | RGRFTVSRDNSKNTLYLQMNSLRAEDTAVYYCA | |
| | TTLCARTDCTWVRSDSWGQGTLVTVSS | |
| SEQ ID NO: 45 | QVQLVESGGGVVQPGRSLRLSCAASGSTLGTYG | 17H9_Hf |
| | MHWVRQAPGKGLEWVAIISPDGGHQDYADPV | |
| | RGRFTISRDNSKNTLYLQMNSLRAEDTAVYYCAT | |
| | TLCARTDCTWVRSDSWGQGTLVTVSS | |
| SEQ ID NO: 46 | ANVLTQSPGTLSLSPGERATLSCRASQRVSGDYL | 17H9_Lg |
| | AWYQQKPGQAPRLLIYGASSRATGIPDRFSGSG | |
| | SGTDFTLTISRLEPEDFAVYYCQHYNGPPVTFGQ | |
| | GTKLEIK | |
| SEQ ID NO: 47 | EIVLTQSPGTLSLSPGERATLSCRASQRVSGDYLA | 17H9_Lf |
| | WYQQKPGQAPRLLIYGASSRATGIPDRFSGSGS | |
| | GTDFTLTISRLEPEDFAVYYCQHYNGPPVTFGQ | |
| | GTKLEIK | |
| SEQ ID NO: 48 | ENVLTQSPGTLSLSPGERATLSCRASQRVSGDYL | 17H9_Lg_A1E |
| | AWYQQKPGQAPRLLIYGASSRATGIPDRFSGSG | |
| | SGTDFTLTISRLEPEDFAVYYCQHYNGPPVTFGQ | |
| | GTKLEIK | |
| SEQ ID NO: 49 | AIVLTQSPGTLSLSPGERATLSCRASQRVSGDYLA | 17H9_Lg_N2I |
| | WYQQKPGQAPRLLIYGASSRATGIPDRFSGSGS | |
| | GTDFTLTISRLEPEDFAVYYCQHYNGPPVTFGQ | |
| | GTKLEIK | |
| SEQ ID NO: 50 | EVQLVESGGGLVQPGGSLRLSCAASGFIFSDYN | 2F8_Hg_M115Y |
| | MNWVRQAPGKGLEWVSSITRSSRTIYYADSVKG | |
| | RFTISRDNAKNSLYLQMNSLRDEDTAVYYCARE | |
| | DFDVSTGPYYYDVWGKGTTVTVSS | |

CDR engineering

| | | |
|---|---|---|
| SEQ ID NO: 51 | EDFNVYTGPYYYDV | 2F8 CDRH3 variant |
| SEQ ID NO: 52 | MASNRAS | 7G6 CDRL2 variant |
| SEQ ID NO: 53 | EVQLVESGGGLVQPGGSLRLSCAASGFIFSDYN | 2F8 VH variant |
| | MNWVRQAPGKGLEWVSSITRSSRTIYYADSVKG | |
| | RFTISRDNAKNSLYLQMNSLRDEDTAVYYCARE | |
| | DFNVYTGPYYYDVWGKGTTVTVSS | |
| SEQ ID NO: 54 | EIVLTQSPLSLPVTPGEPASISCRSSQSLVHRNGYN | 7G6 VL variant |
| | YLDWYLQKPGQSPQLLIYMASNRASGVPDRFS | |
| | GSGSGTDFTLKISRVEAEDVGVYYCMQALQTW | |
| | TFGQGTKVEIK | |

Peanut allergens

| | | |
|---|---|---|
| SEQ ID NO: 55 | RQQWELQGDRRCQSQLERANLRPCEQHLMQK | Ara h 2.0101 |
| | IQRDEDSY | |
| | GRDPYSPSQDPYSPSPYDRRGAGSSQHQERCC | |
| | NELNEFENNQRCMCEALQQIMENQSDRL | |
| | QGRQQEQQFKRELRNLPQQCGLRAPQRCDLE | |
| | VESGGRDRY | |
| SEQ ID NO: 56 | RQQWELQGD RRCQSQLERA NLRPCEQHLM | Ara h 2.0201 |
| | QKIQRDEDSY GRDPYSPSQD PYSPSQDPDR | |
| | RDPYSPSPYD RRGAGSSQHQ ERCCNELNEF | |
| | ENNQRCMCEA LQQIMENQSD | |
| | RLQGRQQEQQ FKRELRNLPQ QCGLRAPQRC | |
| | DLEVESGGRD RY | |
| SEQ ID NO: 57 | RQQPEENACQ FQRLNAQRPD NRIESEGGYI | Ara h 3.0101 |
| | ETWNPNNQEF ECAGVALSRL VLRRNALRRP | |
| | FYSNAPQEIF IQQGRGYFGL IFPGCPRHYE | |
| | EPHTQGRRSQ SQRPPRRLQG EDQSQQQRDS | |
| | HQKVHRFDEG DLIAVPTGVA FWLYNDHDTD | |
| | VVAVSLTDTN NNDNQLDQFP RRENLAGNTE | |
| | QEFLRYQQQS RQSRRRSLPY SPYSPQSQPR | |
| | QEEREFSPRG QHSRRERAGQ EEENEGGNIF | |
| | SGFTPEFLEQ AFQVDDRQIV QNLRGETESE | |
| | EEGAIVTVRG GLRILSPDRK RRADEEEEYD | |
| | EDEYEYDEED RRRGRGSRGR GNGIEETICT | |
| | ASAKKNIGRN RSPDIYNPQA GSLKTANDLN | |
| | LLILRWLGPS AEYGNLYRNA LFVAHYNTNA | |
| | HSIIYRLRGR AHVQVVDSNG NRVYDEELQE | |
| | GHVLVVPQNF AVAGKSQSEN FEYVAFKTDS | |
| | RPSIANLAGE NSVIDNLPEE VVANSYGLQR | |
| | EQARQLKNNN PFKFFVPPSQ QSPRAVA | |
| SEQ ID NO: 58 | MRRERGRQGDSSSCERQVDRVNLKPCEQHIMQ | Ara h 6.0101 |
| | RIMGEQEQYDSYDIRSTRSSDQQQRCCDELNE | |
| | MENTQRCMCEALQQIMENQCDRLQDRQMVQ | |
| | QFKRELMNLPQQCNFRAPQRCDLDVSGGRC | |

Antibody constant regions

| | | |
|---|---|---|
| SEQ ID NO: 59 | ASTKGPSVFPLAPSSKSTSGGTAALGCLVKDYFPE | IgG1 |
| | PVTVSWNSGALTSGVHTFPAVLQSSGLYSLSSVV | |
| | TVPSSSLGTQTYICNVNHKPSNTKVDKKVEPKSC | |
| | DKTHTCPPCPAPELLGGPSVFLFPPKPKDTLMISR | |
| | TPEVTCVVVDVSHEDPEVKFNWYVDGVEVHNA | |
| | KTKPREEQYNSTYRVVSVLTVLHQDWLNGKEYK | |
| | CKVSNKALPAPIEKTISKAKGQPREPQVYTLPPSR | |
| | DELTKNQVSLTCLVKGFYPSDIAVEWESNGQPE | |
| | NNYKTTPPVLDSDGSFFLYSKLTVDKSRWQQG | |
| | NVFSCSVMHEALHNHYTQKSLSLSPGK* | |
| SEQ ID NO: 60 | ASTKGPSVFPLAPCSRSTSESTAALGCLVKDYFPEP | IgG4 S228P |
| | VTVSWNSGALTSGVHTFPAVLQSSGLYSLSSVVT | |
| | VPSSSLGTKTYTCNVDHKPSNTKVDKRVESKYGP | |
| | PCPPCPAPEFLGGPSVFLFPPKPKDTLMISRTPEVT | |
| | CVVVDVSQEDPEVQFNWYVDGVEVHNAKTKP | |
| | REEQFNSTYRVVSVLTVLHQDWLNGKEYKCKVS | |
| | NKGLPSSIEKTISKAKGQPREPQVYTLPPSQEEMT | |
| | KNQVSLTCLVKGFYPSDIAVEWESNGQPENNYK | |
| | TTPPVLDSDGSFFLYSRLTVDKSRWQEGNVFSCS | |
| | VMHEALHNHYTQKSLSLSLGK* | |
| SEQ ID NO: 61 | RTVAAPSVFIFPPSDEQLKSGTASVVCLLNNFYPR | Ckappa |
| | EAKVQWKVDNALQSGNSQESVTEQDSKDSTYS | |
| | LSSTLTLSKADYEKHKVYACEVTHQGLSSPVTKSF | |
| | NRGEC* | |
| SEQ ID NO: 62 | GQPKAAPSVTLFPPSSEELQANKATLVCLISDFYP | Clambda |
| | GAVTVAWKADSSPVKAGVETTTPSKQSNNKYA | |
| | ASSYLSLTPEQWKSHRSYSCQVTHEGSTVEKTVA | |
| SEQ ID NO: 63 | ASTKGPSVFPLAPSSKSTSGGTAALGCLVKDYFPE | CH1 |
| | PVTVSWNSGALTSGVHTFPAVLQSSGLYSLSSVV | |
| | TVPSSSLGTQTYICNVNHKPSNTKVDKKVEPKSC | |
| | * | |

Antibody nucleic acid sequences
17H9

| | | |
|---|---|---|
| SEQ ID NO: 64 | ACCTATGGCATGCAC | CDRH1 |
| SEQ ID NO: 65 | ATTATATCACCTGATGGAGGTCACCAAGACTA | CDRH2 |
| | TGCAGACCCCGTGAGGGGC | |
| SEQ ID NO: 66 | ACGTTGTGTGCTAGGACCGACTGTACATGGG | CDRH3 |
| | TGAGGTCTGACTCC | |
| SEQ ID NO: 67 | AGGGCCAGTCAGAGAGTCAGTGGCGACTAC | CDRL1 |
| | TTAGCC | |
| SEQ ID NO: 68 | GGTGCATCCAGCAGGGCCACT | CDRL2 |
| SEQ ID NO: 69 | CAACACTATAATGGTCCACCCGTCACT | CDRL3 |
| SEQ ID NO: 70 | CAGGTGCAACTGGAGGAGTATGGGGGAGG | VH |
| | CGTGGTCCAGCCTGGGAGGTCCCTGAGACT | |
| | CTCCTGTGTAGCCTCTGGATCCACCTTGGGTA | |
| | CCTATGGCATGCACTGGGTCCGTCAGGCTCC | |
| | AGGCAAGGGGCTGGAGTGGGTGGCAATTAT | |
| | ATCACCTGATGGAGGTCACCAAGACTATGCA | |
| | GACCCCGTGAGGGGCCGATTCACCGTTTCCA | |
| | GAGACAATTCCAAGAATACCCTTTATCTGCAA | |
| | ATGGACAACCTGAGAGTTGAGGACACGGCT | |
| | GTTTATTATTGTGCGACCACGTTGTGTGCTAG | |
| | GACCGACTGTACATGGGTGAGGTCTGACTCC | |
| | TGGGGTCAGGGAACCCTGGTCACCGTCTCCT | |
| | CA | |
| SEQ ID NO: 71 | GCAAATGTGTTGACGCAGTCTCCAGGCACCC | VL |
| | TGTCTTTGTCTCCAGGGGAAAGAGCCACCCT | |
| | CTCCTGCAGGGCCAGTCAGAGAGTCAGTGG | |
| | CGACTACTTAGCCTGGTACCAGCAGAAGCGT | |
| | GGCCAGGCTCCCAGGCTCCTCATCTATGGTG | |
| | CATCCAGCAGGGCCACTGGCATCCCAGACA | |
| | GGTTCAGTGGCAGTGGGTCTGGGACGGACT | |
| | TCACTCTCACCATCAGCAGACTGGAGCCTGA | |
| | AGATTTTGCAGTGTATTACTGTCAACACTATA | |
| | ATGGTCCACCCGTCACTTTTGGCCAGGGGAC | |
| | CAAGCTGGAGATCAAA | |

15E3

| | | |
|---|---|---|
| SEQ ID NO: 72 | ACTTTTTATATGACG | CDRH1 |
| SEQ ID NO: 73 | AATATAAAGCAGGACGGAAGTGAGAAAGAC | CDRH2 |
| | TATCTGGACTCTGTGCGGGGC | |
| SEQ ID NO: 74 | AATGGTCTCCAAGATTCTTACAGTGGTGACTT | CDRH3 |
| | TTTTGACCAC | |
| SEQ ID NO: 75 | TCTGGAACCGGATCCAACATTGGACATAATTA | CDRL1 |
| | TGTCTCC | |
| SEQ ID NO: 76 | GACAATACTAAGCGACCCTCA | CDRL2 |
| SEQ ID NO: 77 | GCAACGTGGGATGCCGACCTGAGTGCTGTG | CDRL3 |
| | CTT | |
| SEQ ID NO: 78 | GAAGTGCAACTGGTGGAGTCGGGGGGAGC | VH |
| | CTTGGTCCAGCCGGGGGGGTCCCTGAGACT | |
| | GTCCTGTGCAGCCTCTGGATTCAAATTTAGCA | |
| | CTTTTTATATGACGTGGATCCGCCAGGCTCCA | |
| | GGGAAGGGCCTGGAGTGGGTGGCCAATATA | |
| | AAGCAGGACGGAAGTGAGAAAGACTATCTG | |
| | GACTCTGTGCGGGGCCGTTTCAACATCTCCA | |
| | GAGACAACGCCAAGAGCGTCCTGTATCTGCA | |
| | GATGAACACCCTGAGCGCCGATGACACGGG | |
| | AGTCTACTATTGTACGAGAAATGGTCTCCAAG | |
| | ATTCTTACAGTGGTGACTTTTTTGACCACTGG | |
| | GGCCAGGGAATCCTGGTCACCGTCTCCTCA | |
| SEQ ID NO: 79 | CAGTCTGTATTGACGCAGCCGCCCTCAATATC | VL |
| | TGCGGCCCCAGGACAGAAGGTCACCATCTCC | |
| | TGCTCTGGAACCGGATCCAACATTGGACATA | |
| | ATTATGTCTCCTGGTACCAACAACTCCCAAGA | |
| | ACAGCCCCCCGACTCCTCATTTTTGACAATAC | |
| | TAAGCGACCCTCAGGCATTCCTGACCGATTCT | |
| | CTGGCTCTAAGTCTGGCTCGTCAGCCACCCT | |
| | GGGCATCACCGGACTCCAGACTGGGGACGA | |
| | GGCCCATTATTTCTGCGCAACGTGGGATGCC | |
| | GACCTGAGTGCTGTGCTTTTCGGCGGCGGG | |
| | ACCAAGCTGACCGTCCTG | |

2F8

| | | |
|---|---|---|
| SEQ ID NO: 80 | GATTATAACATGAAT | CDRH1 |
| SEQ ID NO: 81 | TCCATTACTAGAAGTAGTAGGACCATTTACTA | CDRH2 |
| | CGCAGACTCTGTGAAGGGC | |
| SEQ ID NO: 82 | GAGGATTTCGATGTTTCGACTGGCCCCTACTA | CDRH3 |
| | CATGGACGTC | |
| SEQ ID NO: 83 | AGGGCCAGTCAGAGTGTTAGCAACATGTTCT | CDRL1 |
| | TAGTC | |
| SEQ ID NO: 84 | GGTGCATCTACCAGGGCCACT | CDRL2 |
| SEQ ID NO: 85 | CAGCAGAATGGTAACTCACCATACACT | CDRL3 |
| SEQ ID NO: 86 | GAGGTGCAGCTGGTGGAGTCTGGGGGAGG | VH |
| | CTTGGTCAAGCCGGGGGGGTCGCTGAGACT | |
| | CTCCTGTGCAGCGTCTGGATTCATCTTCAGCG | |
| | ATTATAACATGAATTGGGTCCGCCAGGCTCC | |
| | AGGGAAGGGGCTGGAGTGGGTTTCATCCAT | |
| | TACTAGAAGTAGTAGGACCATTTACTACGCA | |
| | GACTCTGTGAAGGGCCGATTCACCATATCCA | |
| | GAGACAATGCCAAGAACTCACTGCATCTGCA | |
| | AATGAACAGTCTCAGAGACGCGGACACGGC | |
| | TGTGTATTATTGTGCGAGAGAGGATTTCGAT | |
| | GTTTCGACTGGCCCCTACTACATGGACGTCT | |
| | GGGGCAACGGGACCACGGTCATCGTCTCCT | |
| | CA | |
| SEQ ID NO: 87 | GAAATTGTGTTGACGCAGTCTCCAGGCACCC | VL |
| | TGTCTTTGTCTCCAGGGGAAAGAGCCACCCT | |
| | CTCCTGCAGGGCCAGTCAGAGTGTTAGCAAC | |
| | ATGTTCTTAGTCTGGTATCAGCAGAAACCTGG | |
| | CCAGGCTCCCAGGCTCCTCATGTATGGTGCA | |
| | TCTACCAGGGCCACTGACATCCCAGACAGGT | |
| | TCAGTGGCAGTGGGTCTGGGACAGACTTCAC | |
| | TCTCACCATCAGCAGACTGGAGCCTGAAGAT | |
| | TTTGCAGTGTATTACTGTCAGCAGAATGGTAA | |
| | CTCACCATACACTTTTGGCCAGGGGACCAAG | |
| | CTGGAGATCAAA | |
| SEQ ID NO: 88 | gaggatttcAACgttTACactggcccctactactacgacgtc | variant CDRH3 |
| SEQ ID NO: 89 | gaggtgcagctggtggagtctgggggaggcttggtcCagccgggg | variant VH |
| | gggtcgctgagactctcctgtgcagcgtctggattcatcttcagcgat | |
| | tataacatgaattgggtccgccaggctccagggaaggggctggagt | |
| | gggtttcatccattactagaagtagtaggaccatttactacgcagact | |
| | ctgtgaagggccgattcaccatatccagagacaatgccaagaact | |
| | cactgTatctgcaaatgaacagtctcagagacgAggacacggct | |
| | gtgtattattgtgcgagagaggatttcAACgttTACactggcccct | |
| | actactacgacgtctggggcAAGgggaccacggtcaCcgtctc | |
| | ctca | |

7G6

| | | |
|---|---|---|
| SEQ ID NO: 90 | GACTACACTATGCAC | CDRH1 |
| SEQ ID NO: 91 | GCTATATCATATGGTGGGACTAATAAATACTA | CDRH2 |
| | CGCAGATTCCGTGAAGGGC | |
| SEQ ID NO: 92 | GATTCTGGTTATCGGAGTCTTTTGCAC | CDRH3 |
| SEQ ID NO: 93 | AGGTCGAGTCAGAGCCTCGTGCATAGAAATG | CDRL1 |
| | GATACAACTATTTAGAT | |
| SEQ ID NO: 94 | ATGGCTTCTAAACGGGCCTCC | CDRL2 |
| SEQ ID NO: 95 | ATGCAAGCTCTACAAACTTGGACG | CDRL3 |
| SEQ ID NO: 96 | CAGGTGCAGCTGGTGGAGTCTGGGGGAGG | VH |
| | CGTGGTGCAGCCTGGGAGGTCCCTGAGACT | |
| | CTCATGTGCAGCCTCTGGCATCGCCTTCAATG | |
| | ACTACACTATGCACTGGGTCCGCCGGTCTCC | |
| | AGACAAGGGCCTGGAGTGGGTGGCAGCTAT | |
| | ATCATATGGTGGGACTAATAAATACTACGCA | |
| | GATTCCGTGAAGGGCCGATTCACCATCTCCA | |
| | GAGACAGTTCCAAGAACACCCTGTTTCTGCA | |
| | GATGGACAGCCTGAGAGTTGAGGACACGGC | |
| | TGTGTATTACTGTGCGAGAGATTCTGGTTATC | |
| | GGAGTCTTTTGCACTGGGGCCAGGGAACCCT | |
| | GGTCACCGTCTCCTCA | |
| SEQ ID NO: 97 | GAGATTGTGTTGACTCAGTCTCCACTCTCCCT | VL |
| | GCCCGTCACCCCTGGTGAGCCGGCCTCCATC | |
| | TCCTGCAGGTCGAGTCAGAGCCTCGTGCATA | |
| | GAAATGGATACAACTATTTAGATTGGTACCTG | |
| | CAGAAGCCAGGGCAGTCTCCACAGCTCCTGA | |
| | TCTATATGGCTTCTAAACGGGCCTCCGGGGT | |
| | CCCTGACAGGTTCAGTGGCAGTGGGTCAGG | |
| | CACAGAATTTACACTGAAAATCAGCAGAGTG | |
| | GAGGCTGAGGATGTTGGAATTTATTACTGCA | |
| | TGCAAGCTCTACAAACTTGGACGTTCGGCCA | |
| | AGGGACCAAGGTGGAAGTCAAC | |
| SEQ ID NO: 98 | atggcttctaaccgggcctcc | variant CDRL2 |
| SEQ ID NO: 99 | gagattgtgttgactcagtctccactctccttgcccgtcacccctggt | variant VL |
| | gagccggcctccatctcctgcaggtcgagtcagagcctcgtgcata | |
| | gaaatggatacaactatttagattggtacctgcagaagccagggca | |
| | gtctccacagctcctgatctatatggcttctaaccgggcctccggggt | |
| | ccctgacaggttcagtggcagtgggtcaggcacagaTtttacactg | |
| | aaaatcagcagagtggaggctgaggatgttggaGtttattactgcat | |
| | gcaagctctacaaacttggACGTTCGGCCAAGGGACC | |
| | AAGGTGGAAATCAAA | |
| | Grafted fully rabbit antibody constructs (heavy | |
| | and light chain rabbit variable framework | |
| | regions) | |
| SEQ ID NO: 100 | QQLKESGGGLVKPGGSLKLCCKASGFTFSDYNM | 2F8_rb_IGHV1S7_J4; |
| | NWVRQAPGKGLEWIGSITRSSRTIYYADSVKGRF | heavy chain |
| | TLSRDNAQSTVCLQLNSLTAADTATYFCAREDF | |
| | NVYTGPYYYDVWGPGTLVTVSS | |
| SEQ ID NO: 101 | YVMMTQTPSSVSEAVGGTVTIYCRASQSVSNMF | IGKV1S21_JK1-2 |
| | LVWYQQKPGQPPKLLIYGASTRATGVPSRFRGS | (2F8), light chain |
| | GSGTQFTLTISGMKAEDVATYYCQQNGNSPYTF | |
| | GGGTEVVVK | |
| SEQ ID NO: 102 | QEQLVESGGGLVQPEGSLTLTCKASGFDFSDYT | 7G6_rb_IGHV1$47_J3; |
| | MHWVRQAPGKGPEWIAAISYGGTNKYYADSV | heavy chain |
| | KGRFTISRSTSLNTVTLQMTSLTAVDTATYFCARD | |
| | SGYRSLLHWGQGTLVTVSS | |
| SEQ ID NO: 103 | AAVLTQTPSPVSAAVGGTVTIKCRSSQSLVHRN | IGKV1S19_JK1-2 |
| | GYNYLDWYQQKPGQPPKLLIYMASKRASGVPD | (7G6); light chain |
| | RFSGSGSGTQFTLTISGVQCDDAATYYCMQAL | |
| | QTWTFGGGTKVVVK | |
| SEQ ID NO: 104 | AAVLTQTPSPVSAAVGGTVTIKCRSSQSLVHRN | IGKV1S19_JK1-3 |
| | GYNYLDWYQQKPGQPPKLLIYMASKRASGVPD | (7G6); light chain |
| | RFSGSGSGTQFTLTISGVQCDDAATYYCMQAL | |
| | QTWTLGPGTKLEIK | |
| SEQ ID NO: 105 | AAVLTQTPSPVSAAVGGTVTIKCRSSQSLVHRN | K66N_IGKV1S19_JK1-2 |
| | GYNYLDWYQQKPGQPPKLLIYMASNRASGVPD | (7G6); light chain |
| | RFSGSGSGTQFTLTISGVQCDDAATYYCMQAL | |
| | QTWTFGGGTKVVVK | |
| SEQ ID NO: 106 | QQLKESGGGLVKPGGSLKLCCKASGFTFSTYGM | 17H9_rb_IGHV1S7_J3; |
| | HWVRQAPGKGLEWIGIISPDGGHQDYADPVR | heavy chain |
| | GRFTLSRDNAQSTVCLQLNSLTAADTATYFCAR | |
| | TLCARTDCTWVRSDSWGPGTLVTVSS | |
| SEQ ID NO: 107 | DVVMTQTPSPVSAAVGGTVTINCRASQRVSGD | IGKV1S34_JK1-2 |
| | YLAWYQQKPGQPPKLLIYGASSRATGVPSRFSGS | (17H9); light chain |
| | GSGTEFTLTISDLECADAATYYCQHYNGPPVTFG | |
| | GGTEVVVK | |
| SEQ ID NO: 108 | DVVMTQTPSPVSAAVGGTVTINCRASQRVSGD | IGKV1S34_JK1-5 |
| | YLAWYQQKPGQPPKLLIYGASSRATGVPSRFSGS | (17H9); light chain |
| | GSGTEFTLTISDLECADAATYYCQHYNGPPVTFG | |
| | EETKLEIK | |
| SEQ ID NO: 109 | QKQLVESGGGLDQPAGSLKLSCKDSGFTLSTFY | 15E3_rb_IGHV1S8_J5; |
| | MTWVHQAPGKGLEWIANIKQDGSEKDYLDSV | heavy chain |
| | RGRFTISSDNTQNMVDLEMNSLTAADMAIYFCA | |
| | RNGLQDSYSGDFFDHWGQGTLVTVSS | |
| SEQ ID NO: 110 | QPALTQPSSVSGALGGSVTITCSGTGSNIGHNY | IGLV2S2_JL5 (15E3); |
| | VSWYQQLPGSVPKLLMFDNTKRPSGIPERFSGSK | light chain |
| | SGTTASLTISGLQPEDEADYYCATWDADLSAVL | |
| | FGGGTKLTVL | |
| | Grafted human antibody constructs (heavy and | |
| | light chain human variable framework regions) | |
| SEQ ID NO: 111 | EIVLTQSPATLSLSPGERATLSCRSSQSLVHRNGY | 7G6_G1 |
| | NYLDWYQQKPGQAPRLLIYMASKRASGVPARF | |
| | SGSGSGTDFTLTISSLEPEDSALYYCMQALQTWT | |
| | FGGGTKLEIKGGGGSGGGGSGGGGSGGGGSE | |
| | VQLVETGGGLVQPGGSLRLSCAASGFAFNDYT | |
| | MHWVRQAPGKGLEWVSAISYGGTNKYYADSV | |
| | KGRFTISRDNSKDTLYLHMNSLRAEDTAVYYCAR | |
| | DSGYRSLLHWGQGTLVTVSS | |
| SEQ ID NO: 112 | EIVLTQSPLSLPVTLGQPASISCRSSQSLVHRNGY | 7G6_G2 |
| | NYLDWFHQRPGQSPRRLIYMASKRASGVPDRFS | |
| | GSGSGTDFTLKISRVDAEDVGVYYCMQALQTW | |
| | TFGQGTKLTVLGGGGSGGGGSGGGGSGGGG | |
| | SEVQLVETGGGLVQPGGSLRLSCAASGFAFNDY | |
| | TMHWVRQAPGKGLEWVSAISYGGTNKYYADS | |
| | VKGRFTISRDNSKDTLYLHMNSLRAEDTAVYYC | |
| | ARDSGYRSLLHWGQGTLVTVSS | |
| SEQ ID NO: 113 | EIVLTQSPLSLPVTLGQPASISCRSSQSLVHRNGY | 7G6_G4 |
| | NYLDWFHQRPGQSPRRLIYMASKRASGVPDRFS | |
| | GSGSGTDFTLKISRVDAEDVGVYYCMQALQTW | |
| | TFGQGTKLTVLGGGGSGGGGSGGGGSGGGG | |
| | SQVQLVQSGGGLVQPGGSLRLSCAASGFAFND | |
| | YTMHWVRQAPGKGLEWVSAISYGGTNKYYAD | |
| | SVKGRFTISRDNGKKSLYLQMNSLIPEDTALYYC | |
| | AKDSGYRSLLHWGQGTTVTVSS | |
| SEQ ID NO: 114 | EIVLTQSPLSLPVTLGQPASISCRSSQSLVHRNGY | 7G6_G5 |
| | NYLDWFHQRPGQSPRRLIYMASKRASGVPDRFS | |
| | GSGSGTDFTLKISRVDAEDVGVYYCMQALQTW | |
| | TFGQGTKLTVLGGGGSGGGGSGGGGSGGGG | |
| | SQVQLVQSGGGLVQPGGSLRLSCAASGFAFND | |
| | YTMHWVRQAPGKGLEWVSAISYGGTNKYYAD | |
| | SVKGRFTISRDNSKNTLYLQMNSLRAEDTAVYYC | |
| | AADSGYRSLLHWGQGTLVTVSS | |
| SEQ ID NO: 115 | EIVLTQSPLSLPVTLGQPASISCRSSQSLVHRNGY | 7G6_G6 |
| | NYLDWFHQRPGQSPRRLIYMASKRASGVPDRFS | |
| | GSGSGTDFTLKISRVDAEDVGVYYCMQALQTW | |
| | TFGQGTKLTVLGGGGSGGGGSGGGGSGGGG | |
| | SEVQLVESGGGLVQPGGSLRLSCAASGFAFNDY | |
| | TMHWVRQAPGKGLEWVSAISYGGTNKYYADS | |
| | VKGRFTISRDNSKNTLYLQMNSLRAEDTAVYYC | |
| | AKDSGYRSLLHWGQGTLVTVSS | |
| SEQ ID NO: 116 | EIVLTQSPATLSLSPGERATLSCRSSQSLVHRNGY | 7G6_G7 |
| | NYLDWYQQKPGQAPRLLIYMASKRASGVPARF | |
| | SGSGSGTDFTLTISSLEPEDSALYYCMQALQTWT | |
| | FGGGTKLEIKGGGGSGGGGSGGGGSGGGGS | |
| | QVQLVQSGGGLVQPGGSLRLSCAASGFAFNDY | |
| | TMHWVRQAPGKGLEWVSAISYGGTNKYYADS | |
| | VKGRFTISRDNSKNTLYLQMNSLRAEDTAVYYC | |
| | AADSGYRSLLHWGQGTLVTVSS | |
| SEQ ID NO: 117 | EIVLTQSPATLSLSPGERATLSCRSSQSLVHRNGY | 7G6_G8 |
| | NYLDWYQQKPGQAPRLLIYMASKRASGVPARF | |
| | SGSGSGTDFTLTISSLEPEDSALYYCMQALQTWT | |
| | FGGGTKLEIKGGGGSGGGGSGGGGSGGGGSE | |
| | VQLVESGGGLVQPGGSLRLSCAASGFAFNDYT | |
| | MHWVRQAPGKGLEWVSAISYGGTNKYYADSV | |
| | KGRFTISRDNSKNTLYLQMNSLRAEDTAVYYCA | |
| | KDSGYRSLLHWGQGTLVTVSS | |
| SEQ ID NO: 118 | QSVLTQPPSVSAAPGQKVTISCSGTGSNIGHNY | 15E3_G4 |
| | VSWYQQLPGTAPKLLIYDNTKRPSGIPDRFSGSK | |
| | SGTSATLGITGLQTGDEADYYCATWDADLSAVL | |
| | FGTGTKLTVLGGGGSGGGGSGGGGSGGGGS | |
| | QVQLVQSGGGLVQPGGSLRLSCAASGFKFSTFY | |
| | MTWVRQAPGKGLEWVSNIKQDGSEKDYLDSV | |
| | RGRFTISRDNSKNTLYLQMNSLRAEDTAVYYCA | |
| | ANGLQDSYSGDFFDHWGQGTLVTVSS | |
| | Wildtype (wt) sequences | |
| SEQ ID NO: 119 | EVQLVESGGGLVQPGGSLRLSCAASGFIFSDYN | 2F8_65_Hg (heavy |
| | MNWVRQAPGKGLEWVSSITRSSRTIYYADSVKG | chain) |
| | RFTISRDNAKNSLYLQMNSLRDEDTAVYYCARE | |
| | DFNVYTGPYYYDVWGKGTTVTVSS | |
| SEQ ID NO: 120 | EIVLTQSPGTLSLSPGERATLSCRASQSVSNMFLV | 2F8_Lwt (light chain) |
| | WYQQKPGQAPRLLMYGASTRATDIPDRFSGSG | |
| | SGTDFTLTISRLEPEDFAVYYCQQNGNSPYTFGQ | |
| | GTKLEIK | |
| SEQ ID NO: 121 | QVQLVESGGGVVQPGRSLRLSCAASGIAFNDYT | 7G6_Hwt (heavy |
| | MHWVRRSPDKGLEWVAAISYGGTNKYYADSVK | chain) |
| | GRFTISRDSSKNTLFLQMDSLRVEDTAVYYCARD | |
| | SGYRSLLHWGQGTLVTVSS | |
| SEQ ID NO: 122 | EIVLTQSPLSLPVTPGEPASISCRSSQSLVHRNGYN | 7G6_Lwt (light chain) |
| | YLDWYLQKPGQSPQLLIYMASKRASGVPDRFSG | |
| | SGSGTEFTLKISRVEAEDVGIYYCMQALQTWTFG | |
| | QGTKVEVN | |
| SEQ ID NO: 123 | QVQLEEYGGGVVQPGRSLRLSCVASGSTLGTYG | 17H9_Hwt (heavy |
| | MHWVRQAPGKGLEWVAIISPDGGHQDYADPV | chain) |
| | RGRFTVSRDNSKNTLYLQMDNLRVEDTAVYYC | |
| | ATTLCARTDCTWVRSDSWGQGTLVTVSS | |
| SEQ ID NO: 124 | ANVLTQSPGTLSLSPGERATLSCRASQRVSGDYL | 17H9_Lwt (light |
| | AWYQQKRGQAPRLLIYGASSRATGIPDRESGSG | |
| | SGTDFTLTISRLEPEDFAVYYCQHYNGPPVTFGQ | chain) |
| | GTKLEIK | |
| SEQ ID NO: 125 | EVQLVESGGALVQPGGSLRLSCAASGFKFSTFYM | 15E3_Hwt (heavy |
| | TWIRQAPGKGLEWVANIKQDGSEKDYLDSVRG | chain) |
| | RFNISRDNAKSVLYLQMNTLSADDTGVYYCTRN | |
| | GLQDSYSGDFFDHWGQGILVTVSS | |
| SEQ ID NO: 126 | QSVLTQPPSISAAPGQKVTISCSGTGSNIGHNYV | 15E3_Lwt (light |
| | SWYQQLPRTAPRLLIFDNTKRPSGIPDRFSGSKS | chain) |
| | GSSATLGITGLQTGDEAHYFCATWDADLSAVLF | |
| | GGGTKLTVL | |
| SEQ ID NO: 127 | EIVLTQSPLSLPVTPGEPASISCRSSQSLVHRNGYN | 7G6_wt |
| | YLDWYLQKPGQSPQLLIYMASKRASGVPDRFSG | |
| | SGSGTEFTLKISRVEAEDVGIYYCMQALQTWTFG | |
| | QGTKVEVNGGGGSGGGGGGGGSGGGGSQ | |
| | VQLVESGGGVVQPGRSLRLSCAASGIAFNDYTM | |
| | HWVRRSPDKGLEWVAAISYGGTNKYYADSVKG | |
| | RFTISRDSSKNTLFLQMDSLRVEDTAVYYCARDS | |
| | GYRSLLHWGQGTLVTVSS | |
| SEQ ID NO: 128 | QSVLTQPPSISAAPGQKVTISCSGTGSNIGHNYV | 15E3_wt |
| | SWYQQLPRTAPRLLIFDNTKRPSGIPDRFSGSKS | |
| | GSSATLGITGLQTGDEAHYFCATWDADLSAVLF | |
| | GGGTKLTVLGGGGGSGGGGSGGGGSGGGGS | |
| | EVQLVESGGALVQPGGSLRLSCAASGFKFSTFYM | |
| | TWIRQAPGKGLEWVANIKQDGSEKDYLDSVRG | |
| | RFNISRDNAKSVLYLQMNTLSADDTGVYYCTRN | |
| | GLQDSYSGDFFDHWGQGILVTVSS | |

1. An antibody, or an antigen-binding fragment thereof, which binds specifically to Ara h 2 (Arachis hypogaea allergen 2).

2. The antibody, or an antigen-binding fragment thereof, according to claim 1, wherein the antibody or the antigen-binding fragment thereof, further binds specifically to Ara h 3 or to Ara h 6.

3. The antibody, or an antigen-binding fragment thereof, according to claim 1 or 2, wherein the antibody, or the antigen-binding fragment thereof, comprises a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6.

4. The antibody, or an antigen-binding fragment thereof, according to any one of the previous claims, wherein the antibody, or the antigen-binding fragment thereof, comprises a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6.

5. The antibody, or an antigen-binding fragment thereof, according to any one of the previous claims, wherein the antibody, or the antigen-binding fragment thereof, comprises a VH having at least 70% identity to any one of SEQ ID NOs 7, 44 and 45; and a VL having at least 70% identity to any one of SEQ ID NOs 8, 46, 47, 48 and 49.

6. The antibody, or an antigen-binding fragment thereof, according to any one of the previous claims, wherein the antibody, or the antigen-binding fragment thereof, comprises a VH according to any one of SEQ ID NOs 7, 44 and 45; and a VL according to any one of SEQ ID NOs 8, 46, 47, 48 and 49.

7. The antibody, or an antigen-binding fragment thereof, according to claim 1 or 2, wherein the antibody, or the antigen-binding fragment thereof, comprises a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14.

8. The antibody, or an antigen-binding fragment thereof, according to any one of claims 1, 2 and 7, wherein the antibody, or the antigen-binding fragment thereof, comprises a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14.

9. The antibody, or an antigen-binding fragment thereof, according to any one of claims 1, 2, 7 and 8, wherein the antibody, or the antigen-binding fragment thereof, comprises a VH having at least 70% identity to any one of SEQ ID NOs 15, 37 and 38; and a VL having at least 70% identity to any one of SEQ ID NOs 16, 39 and 40.

10. The antibody, or an antigen-binding fragment thereof, according to any one of claims 1, 2 and 7-9, wherein the antibody, or the antigen-binding fragment thereof, comprises a VH according to any one of SEQ ID NOs 15, 37 and 38; and a VL according to any one of SEQ ID NOs 16, 39 and 40.

11. The antibody, or an antigen-binding fragment thereof, according to claim 1 or 2, wherein the antibody, or the antigen-binding fragment thereof, comprises a CDRH1 having at least 70% identity to SEQ ID NO: 17, a CDRH2 having at least 70% identity to SEQ ID NO: 18, a CDRH3 having at least 70% identity to SEQ ID NO: 51, a CDRL1 having at least 70% identity to SEQ ID NO: 20, a CDRL2 having at least 70% identity to SEQ ID NO: 21, and a CDRL3 having at least 70% identity to SEQ ID NO: 22.

12. The antibody, or an antigen-binding fragment thereof, according to any one of claims 1, 2 and 11, wherein the antibody, or the antigen-binding fragment thereof, comprises a CDRH1 according to SEQ ID NO: 17, a CDRH2 according to SEQ ID NO: 18, a CDRH3 according to SEQ ID NO: 51, a CDRL1 according to SEQ ID NO: 20, a CDRL2 according to SEQ ID NO: 21, and a CDRL3 according to SEQ ID NO: 22.

13. The antibody, or an antigen-binding fragment thereof, according to any one of claims 1, 2, 11 and 12, wherein the antibody, or the antigen-binding fragment thereof, comprises a VH having at least 70% identity to SEQ ID NO: 53; and a VL having at least 70% identity to any one of SEQ ID NOs 24, 42 and 43.

14. The antibody, or an antigen-binding fragment thereof, according to any one of claims 1, 2 and 11-13, wherein the antibody, or the antigen-binding fragment thereof, comprises a VH according to SEQ ID NO: 53; and a VL according to any one of SEQ ID NOs 24, 42 and 43.

15. The antibody, or an antigen-binding fragment thereof, according to claim 1 or 2, wherein the antibody, or the antigen-binding fragment thereof, comprises a VH having at least 70% identity to any one of SEQ ID NOs 41, 50 and 53; and a VL having at least 70% identity to SEQ ID NO: 42 or 43.

16. The antibody, or an antigen-binding fragment thereof, according to claim 15, wherein the antibody, or the antigen-binding fragment thereof, comprises a VH according to any one of SEQ ID NOs 41, 50 and 53; and a VL according to SEQ ID NO: 42 or 43.

17. The antibody, or an antigen-binding fragment thereof, according to claim 1 or 2, wherein the antibody, or the antigen-binding fragment thereof, comprises a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 52, and a CDRL3 having at least 70% identity to SEQ ID NO: 30.

18. The antibody, or an antigen-binding fragment thereof, according to any one of claims 1, 2 and 17, wherein the antibody, or the antigen-binding fragment thereof, comprises a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 52, and a CDRL3 according to SEQ ID NO: 30.

19. The antibody, or an antigen-binding fragment thereof, according to any one of claims 1, 2, 17 and 18, wherein the antibody, or the antigen-binding fragment thereof, comprises a VH having at least 70% identity to any one of SEQ ID NOs 31, 33 and 34; and a VL having at least 70% identity to SEQ ID NO: 54.

20. The antibody, or an antigen-binding fragment thereof, according to any one of claims 1, 2 and 17-19, wherein the antibody, or the antigen-binding fragment thereof, comprises a VH according to any one of SEQ ID NOs 31, 33 and 34; and a VL according to SEQ ID NO: 54.

21. The antibody, or an antigen-binding fragment thereof, according to claim 1 or 2, wherein the antibody, or the antigen-binding fragment thereof, comprises a VH having at least 70% identity to SEQ ID NO: 33 or 34; and a VL having at least 70% identity to any one of SEQ ID NOs 35, 36 and 54.

22. The antibody, or an antigen-binding fragment thereof, according to claim 21, wherein the antibody, or the antigen-binding fragment thereof, comprises a VH according to SEQ ID NO: 33 or 34; and a VL according to any one of SEQ ID NOs 35, 36 and 54.

23. The antibody, or an antigen-binding fragment thereof, according to any one of the previous claims, wherein the CDRs or the variable regions of the antibody, or the antigen-binding fragment thereof, are human or are derived from human CDR or variable region sequences.

24. The antibody, or an antigen-binding fragment thereof, according to any one of the previous claims, wherein the antibody, or the antigen-binding fragment thereof, is a human antibody.

25. The antibody, or an antigen-binding fragment thereof, according to any one of the previous claims, wherein the antibody, or the antigen-binding fragment thereof, is a monoclonal antibody.

26. The antibody according to any one according to the previous claims, wherein the antibody comprises an Fc moiety.

27. The antibody according to any one of the previous claims, wherein the antibody is of the IgG or IgA type.

28. The antibody according to claim 27, wherein the antibody is of the IgG1 or IgG4 type.

29. The antibody according to any one according to the previous claims, wherein the variable regions or the CDRs are derived from an IgE antibody and grafted in a scaffold of an IgG or IgA antibody.

30. The antibody, or an antigen-binding fragment thereof, according to any one of the previous claims, wherein the antibody, or the antigen-binding fragment thereof, is purified.

31. The antibody, or an antigen-binding fragment thereof, according to any one of the previous claims, wherein the antibody, or the antigen-binding fragment thereof, is a single-chain antibody.

32. The antibody, or an antigen-binding fragment thereof, according to claim 24, wherein the antibody, or the antigen-binding fragment thereof, is an scFv.

33. The antibody, or an antigen-binding fragment thereof, according to any one of claims 1 to 23, wherein the antibody, or the antigen-binding fragment thereof, is selected from Fab, Fab′, F(ab′)2 and Fv.

34. The antibody, or an antigen-binding fragment thereof, according to any one of the previous claims, wherein the antibody, or the antigen-binding fragment thereof, is a multispecific antibody or a multispecific antigen-binding fragment.

35. The antibody, or an antigen-binding fragment thereof, according to claim 34, wherein the antibody, or the antigen-binding fragment thereof, binds to distinct, non-overlapping epitopes of Ara h 2.

36. The antibody, or an antigen-binding fragment thereof, according to claim 34, wherein the antibody, or the antigen-binding fragment thereof, comprises at least two of the following:
(i) an antigen-binding site comprising (a) the CDRH1, CDRH2, CDRH3, CDRL1, CDRL2 and CDRL3 sequences and/or (b) the VH and VL sequences as defined in any one of claims 3 to 6;
(ii) an antigen-binding site comprising (a) the CDRH1, CDRH2, CDRH3, CDRL1, CDRL2 and CDRL3 sequences and/or (b) the VH and VL sequences as defined in any one of claims 7 to 10;
(iii) an antigen-binding site comprising (a) the CDRH1, CDRH2, CDRH3, CDRL1, CDRL2 and CDRL3 sequences and/or (b) the VH and VL sequences as defined in any one of claims 11 to 16;
(iv) an antigen-binding site comprising (a) the CDRH1, CDRH2, CDRH3, CDRL1, CDRL2 and CDRL3 sequences and/or (b) the VH and VL sequences as defined in any one of claims 17 to 22.

37. A multispecific antibody, or a multispecific antigen-binding fragment, which binds to distinct, non-overlapping epitopes of Ara h 2, wherein the antibody, or the antigen-binding fragment, comprises at least two of the following:
(i) an antigen-binding site comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
(ii) an antigen-binding site comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
(iii) an antigen-binding site comprising a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
(iv) an antigen-binding site comprising a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30.

38. The antibody, or the antigen-binding fragment according to claim 37, wherein the antibody, or the antigen-binding fragment, comprises at least two of the following:
(i) an antigen-binding site comprising a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antigen-binding site comprising a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
(iii) an antigen-binding site comprising a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
(iv) an antigen-binding site comprising a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30.

39. The antibody or the antigen-binding fragment according to claim 37 or 38, wherein the antibody, or the antigen-binding fragment, comprises at least two of the following:
(i) an antigen-binding site comprising a VH having at least 70% identity to any one of SEQ ID NOs 7, 44 and 45; and a VL having at least 70% identity to any one of SEQ ID NOs 8, 46, 47, 48 and 49;
(ii) an antigen-binding site comprising a VH having at least 70% identity to any one of SEQ ID NOs 15, 37 and 38; and a VL having at least 70% identity to any one of SEQ ID NOs 16, 39 and 40;
(iii) an antigen-binding site comprising a VH having at least 70% identity to any one of SEQ ID NOs 23, 41, 50 and 53; and a VL having at least 70% identity to any one of SEQ ID NOs 24, 42 and 43;
(iv) an antigen-binding site comprising a VH having at least 70% identity to any one of SEQ ID NOs 31, 33 and 34; and a VL having at least 70% identity to any one of SEQ ID NOs 32, 35, 36 and 54.

40. The antibody or the antigen-binding fragment according to any one of claims 37-39, wherein the antibody, or the antigen-binding fragment, comprises at least two of the following:
(i) an antigen-binding site comprising a VH according to any one of SEQ ID NOs 7, 44 and 45; and a VL according to any one of SEQ ID NOs 8, 46, 47, 48 and 49;
an antigen-binding site comprising a VH according to any one of SEQ ID NOS (ii) 15, 37 and 38; and a VL according to any one of SEQ ID NOs 16, 39 and 40;
(iii) an antigen-binding site comprising a VH according to any one of SEQ ID NOS 23, 41, 50 and 53; and a VL according to any one of SEQ ID NOs 24, 42 and 43;
(iv) an antigen-binding site comprising a VH according to any one of SEQ ID NOS 31, 33 and 34; and a VL according to any one of SEQ ID NOs 32, 35, 36 and 54.

41. The antibody or the antigen-binding fragment according to any one of claims 36-39, wherein the antibody, or the antigen-binding fragment, is trispecific and comprises three distinct antigen-binding sites selected from (i)-(iv).

42. The antibody or the antigen-binding fragment according to any one of claims 36-39, wherein the antibody, or the antigen-binding fragment, is tetraspecific and comprises the four distinct antigen-binding sites according to (i), (ii), (iii) and (iv).

43. The antibody or the antigen-binding fragment according to claim 3 or 4, wherein the CDRH1, CDRH2 and CDRH3 sequences are contained by a human variable light chain framework region VH chain selected from the group consisting of the amino acid sequences encoded by the genes IGHV1-18, IGHV1-2, IGHV1-24, IGHV1-3, IGHV1-45, IGHV1-46, IGHV1-58, IGHV1-69, IGHV1-69-2, IGHV1-69D, IGHV1-8, IGHV2-26, IGHV2-5, IGHV2-70, IGHV2-70D, IGHV3-11, IGHV3-13, IGHV3-15, IGHV3-20, IGHV3-21, IGHV3-23, IGHV3-23D, IGHV3-30, IGHV3-30-3, IGHV3-30-5, IGHV3-33, IGHV3-35, IGHV3-43, IGHV3-43D, IGHV3-48, IGHV3-49, IGHV3-53, IGHV3-62, IGHV3-64, IGHV3-64D, IGHV3-66, IGHV3-7, IGHV3-72, IGHV3-73, IGHV3-74, IGHV3-9, IGHV3-NL1, IGHV4-28, IGHV4-30-1, IGHV4-30-2, IGHV4-30-4, IGHV4-31, IGHV4-34, and IGHV4-38-2.

44. The antibody or the antigen-binding fragment according to claim 3, 4 or 43, wherein the CDRL1, CDRL2 and CDRL3 sequences are contained by a human variable light chain framework region VL chain selected from the group consisting of the amino acid sequences encoded by the genes IGLV1-36, IGLV1-40, IGLV1-44, IGLV1-47, IGLV1-51, IGLV10-54, IGLV2-11, IGLV2-14, IGLV2-18, IGLV2-23, IGLV2-8, IGLV3-1, IGLV3-10, IGLV3-12, IGLV3-16, IGLV3-19, IGLV3-21, IGLV3-22, IGLV3-25, IGLV3-27, IGLV3-9, IGLV4-3, IGLV4-60, IGLV4-69, IGLV5-37, IGLV5-39, IGLV5-45, IGLV5-52, IGLV6-57, IGLV7-43, IGLV7-46, IGLV8-61, and IGLV9-49.

45. The antibody or the antigen-binding fragment according to claim 3, 4, or 43, wherein the CDRL1, CDRL2 and CDRL3 sequences are contained by a human variable light chain framework region VK chain selected from the group consisting of the amino acid sequences encoded by the genes IGKV1-12, IGKV1-13, IGKV1-16, IGKV1-17, IGKV1-27, IGKV1-33, IGKV1-39, IGKV1-5, IGKV1-6, IGKV1-8, IGKV1-9, IGKV1-NL1, IGKV1D-12, IGKV1D-13, IGKV1D-16, IGKV1D-17, IGKV1D-33, IGKV1D-39, IGKV1D-43, IGKV1D-8, IGKV2-24, IGKV2-28, IGKV2-29, IGKV2-30, IGKV2-40, IGKV2D-26, IGKV2D-28, IGKV2D-29, IGKV2D-30, IGKV2D-40, IGKV3-11, IGKV3-15, IGKV3-20, IGKV3D-11, IGKV3D-15, IGKV3D-20, IGKV3D-7, IGKV4-1, IGKV5-2, IGKV6-21, and IGKV6D-21.

46. The antibody, or an antigen-binding fragment thereof, according to any one of the previous claims for use as a medicament.

47. The antibody, or an antigen-binding fragment thereof, for use according to claim 46 in prophylaxis or treatment of peanut allergy.

48. A nucleic acid molecule comprising a polynucleotide encoding the antibody, or an antigen-binding fragment thereof, according to any one of claims 1 to 45.

49. The nucleic acid molecule according to claim 48 comprising a nucleic acid sequence as set forth in any one of SEQ ID NOs 64-99; or a sequence variant thereof having at least 50%, at least 55%, at least 60%, at least 65%, at least 70%, at least 75%, at least 80%, at least 85%, at least 88%, at least 90%, at least 92%, at least 95%, at least 96%, at least 97%, at least 98% or at least 99% sequence identity.

50. A plurality of nucleic acid molecules encoding the antibody, or an antigen-binding fragment thereof, according to any one of claims 1 to 45, wherein each of the nucleic acid molecules comprises a polynucleotide encoding an immunoglobulin chain of the antibody, or an antigen-binding fragment thereof.

51. The combination of nucleic acid molecules according to claim 50 comprising a nucleic acid sequence as set forth in any one of SEQ ID NOs 64-99; or a sequence variant thereof having at least 50%, at least 55%, at least 60%, at least 65%, at least 70%, at least 75%, at least 80%, at least 85%, at least 88%, at least 90%, at least 92%, at least 95%, at least 96%, at least 97%, at least 98% or at least 99% sequence identity.

52. A vector comprising the nucleic acid molecule according to claim 48 or 49 or the plurality of nucleic acid molecules according to claim 50 or 51.

53. A plurality of vectors comprising the plurality of nucleic acid molecules according to claim 50 or 51.

54. A host cell expressing the antibody, or an antigen-binding fragment thereof, according to any one of claims 1 to 45, or comprising the vector according to claim 52 or the combination of vectors of claim 53.

55. A method for preparing the antibody, or an antigen-binding fragment thereof, according any one of claims 1 to 45, or immunoglobulin chain(s) thereof, said method comprising
(i) culturing the host cell according to claim 54; and
(ii) isolating the antibody or immunoglobulin chain(s) thereof from the culture.

56. A composition comprising the antibody, or an antigen-binding fragment thereof, according to any one of claims 1 to 45, the nucleic acid or the plurality of nucleic acids according to any one of claims 48 to 51, the vector or the plurality of vectors according to claim 52 or 53, or the cell according to claim 54.

57. The composition according to claim 56 further comprising a pharmaceutically acceptable excipient, diluent or carrier.

58. The composition according to claim 56 or 57, wherein the composition comprises at least two distinct antibodies, or antigen-binding fragments thereof, binding to distinct, non-overlapping epitopes of Ara h 2.

59. The composition according to claim 58, wherein the composition comprises at least two of the following:
(i) the antibody, or the antigen-binding fragment thereof, according to any one of claims 3 to 6;
(ii) the antibody, or the antigen-binding fragment thereof, according to any one of claims 7 to 10;
(iii) the antibody, or the antigen-binding fragment thereof, according to any one of claims 11 to 16;
(iv) the antibody, or the antigen-binding fragment thereof, according to any one of claims 17 to 22.

60. The composition according to claim 58 or 59 comprising (exactly) three or four distinct antibodies, or antigen-binding fragments, binding to distinct, non-overlapping epitopes of Ara h 2, wherein the (exactly) three or four distinct antibodies, or antigen-binding fragments, are preferably selected from (i)-(iv) of claim 59.

61. A composition comprising at least three distinct antibodies, or antigen-binding fragments thereof, wherein the three distinct antibodies or antigen-binding fragments bind to distinct, non-overlapping epitopes of Ara h 2, wherein the composition comprises at least three of the following:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
(iii) an antibody, or an antigen-binding fragment thereof, comprising a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
(iv) an antibody, or an antigen-binding fragment thereof, comprising a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30.

62. The composition according to claim 61, wherein the composition comprises at least three of the following:
(i) an antibody, or an antigen-binding fragment thereof, comprising a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
(iii) an antibody, or an antigen-binding fragment thereof, comprising a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
(iv) an antibody, or an antigen-binding fragment thereof, comprising a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30.

63. The composition according to claim 61 or 62, wherein the composition comprises at least three of the following:
(i) an antibody, or an antigen-binding fragment thereof, comprising a VH having at least 70% identity to any one of SEQ ID NOs 7, 44 and 45; and a VL having at least 70% identity to any one of SEQ ID NOs 8, 46, 47, 48 and 49;
(ii) an antibody, or an antigen-binding fragment thereof, comprising a VH having at least 70% identity to any one of SEQ ID NOs 15, 37 and 38; and a VL having at least 70% identity to any one of SEQ ID NOs 16, 39 and 40;
(iii) an antibody, or an antigen-binding fragment thereof, comprising a VH having at least 70% identity to any one of SEQ ID NOs 23, 41, 50 and 53; and a VL having at least 70% identity to any one of SEQ ID NOs 24, 42 and 43;
(iv) an antibody, or an antigen-binding fragment thereof, comprising a VH having at least 70% identity to any one of SEQ ID NOs 31, 33 and 34; and a VL having at least 70% identity to any one of SEQ ID NOs 32, 35, 36 and 54.

64. The composition according to any one of claims 61-63, wherein the composition comprises at least three of the following:
(i) an antibody, or an antigen-binding fragment thereof, comprising a VH according to any one of SEQ ID NOs 7, 44 and 45; and a VL according to any one of SEQ ID NOs 8, 46, 47, 48 and 49;
(ii) an antibody, or an antigen-binding fragment thereof, comprising a VH according to any one of SEQ ID NOs 15, 37 and 38; and a VL according to any one of SEQ ID NOs 16, 39 and 40;
(iii) an antibody, or an antigen-binding fragment thereof, comprising a VH according to any one of SEQ ID NOs 23, 41, 50 and 53; and a VL according to any one of SEQ ID NOs 24, 42 and 43;
(iv) an antibody, or an antigen-binding fragment thereof, comprising a VH according to any one of SEQ ID NOs 31, 33 and 34; and a VL according to any one of SEQ ID NOs 32, 35, 36 and 54.

65. The composition according to any one of claims 59-64, wherein the composition comprises the four distinct antibodies, or antigen-binding fragments, according to (i), (ii), (iii) and (iv).

66. The composition according to any one of claims 56-64, wherein the composition further comprises at least one additional agent useful for treating peanut allergy.

67. The composition according to claim 66, wherein the additional agent useful for treating peanut allergy is selected from the group consisting of: β-adrenergic agonists, epinephrine, antihistamine, corticosteroid, anti-IgE antibody, anti-IgE antibody binding fragment, peptide vaccine and further antibodies capable of binding to a peanut allergen.

68. The composition according to any one of claims 56-67, wherein the composition further comprises a peanut allergen, preferably selected from the group consisting of Ara h1, Ara h2, Ara h3 and Ara h6 or a mixture thereof.

69. A kit comprising one or more of
(i) the antibody, or an antigen-binding fragment thereof, according to any one of claims 1 to 45;
(ii) the nucleic acid molecule(s) according to any one of claims 48 to 51;
(iii) the vector(s) according to claim 52 or 53;
(iv) the cell according to claim 54; and/or
(v) the composition according to any one of claims 56 to 68.

70. The kit according to claim 69, wherein the kit comprises
(i) at least two distinct antibodies, or antigen-binding fragments thereof, according to any one of claims 1 to 45, which bind to distinct, non-overlapping epitopes of Ara h 2;
(ii) nucleic acid molecule(s) encoding at least two distinct antibodies, or antigen-binding fragments thereof, according to any one of claims 1 to 45, which bind to distinct, non-overlapping epitopes of Ara h 2; or
(iii) composition(s) comprising (i) or (ii).

71. The kit according to claim 70 comprising three or four distinct antibodies, or antigen-binding fragments thereof, according to any one of claims 1 to 45, which bind to distinct, non-overlapping epitopes of Ara h 2; or nucleic acid(s) encoding said antibodies or compositions comprising said antibodies.

72. A kit comprising at least three distinct antibodies, or antigen-binding fragments thereof, wherein the three distinct antibodies or antigen-binding fragments bind to distinct, non-overlapping epitopes of Ara h 2, wherein the kit comprises at least three of the following:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
(iii) an antibody, or an antigen-binding fragment thereof, comprising a CDRH1 having at least 70% identity to according to SEQ ID NO: 17, a CDRH2 having at least 70% identity to SEQ ID NO: 18, a CDRH3 having at least 70% identity to SEQ ID NO: 19 or 51, a CDRL1 having at least 70% identity to SEQ ID NO: 20, a CDRL2 having at least 70% identity to SEQ ID NO: 21, and a CDRL3 having at least 70% identity to SEQ ID NO: 22;
(iv) an antibody, or an antigen-binding fragment thereof, comprising a CDRH1 having at least 70% identity to SEQ ID NO: 25, a CDRH2 having at least 70% identity to SEQ ID NO: 26, a CDRH3 having at least 70% identity to SEQ ID NO: 27, a CDRL1 having at least 70% identity to SEQ ID NO: 28, a CDRL2 having at least 70% identity to SEQ ID NO: 29 or 52, and a CDRL3 having at least 70% identity to SEQ ID NO: 30.

73. The kit according to claim 72, wherein the kit comprises at least three of the following:
(i) an antibody, or an antigen-binding fragment thereof, comprising a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
(iii) an antibody, or an antigen-binding fragment thereof, comprising a CDRH1 according to according to SEQ ID NO: 17, a CDRH2 according to SEQ ID NO: 18, a CDRH3 according to SEQ ID NO: 19 or 51, a CDRL1 according to SEQ ID NO: 20, a CDRL2 according to SEQ ID NO: 21, and a CDRL3 according to SEQ ID NO: 22;
(iv) an antibody, or an antigen-binding fragment thereof, comprising a CDRH1 according to SEQ ID NO: 25, a CDRH2 according to SEQ ID NO: 26, a CDRH3 according to SEQ ID NO: 27, a CDRL1 according to SEQ ID NO: 28, a CDRL2 according to SEQ ID NO: 29 or 52, and a CDRL3 according to SEQ ID NO: 30.

74. The kit according to claim 72 or 73, wherein the kit comprises at least three of the following:
(i) an antibody, or an antigen-binding fragment thereof, comprising a VH having at least 70% identity to any one of SEQ ID NOs 7, 44 and 45; and a VL having at least 70% identity to any one of SEQ ID NOs 8, 46, 47, 48 and 49;
(ii) an antibody, or an antigen-binding fragment thereof, comprising a VH having at least 70% identity to any one of SEQ ID NOs 15, 37 and 38; and a VL having at least 70% identity to any one of SEQ ID NOs 16, 39 and 40;
(iii) an antibody, or an antigen-binding fragment thereof, comprising a VH having at least 70% identity to any one of SEQ ID NOs 23, 41, 50 and 53; and a VL having at least 70% identity to any one of SEQ ID NOs 24, 42 and 43;
(iv) an antibody, or an antigen-binding fragment thereof, comprising a VH having at least 70% identity to any one of SEQ ID NOs 31, 33 and 34; and a VL having at least 70% identity to any one of SEQ ID NOs 32, 35, 36 and 54.

75. The kit according to any one of claims 72-74, wherein the kit comprises at least three of the following:
(i) an antibody, or an antigen-binding fragment thereof, comprising a VH according to any one of SEQ ID NOs 7, 44 and 45; and a VL according to any one of SEQ ID NOs 8, 46, 47, 48 and 49;
(ii) an antibody, or an antigen-binding fragment thereof, comprising a VH according to any one of SEQ ID NOs 15, 37 and 38; and a VL according to any one of SEQ ID NOs 16, 39 and 40;
(iii) an antibody, or an antigen-binding fragment thereof, comprising a VH according to any one of SEQ ID NOs 23, 41, 50 and 53; and a VL according to any one of SEQ ID NOs 24, 42 and 43;
(iv) an antibody, or an antigen-binding fragment thereof, comprising a VH according to any one of SEQ ID NOs 31, 33 and 34; and a VL according to any one of SEQ ID NOs 32, 35, 36 and 54.

76. The kit according to any one of claims 72-75, wherein the kit comprises the four distinct antibodies, or antigen-binding fragments, according to (i), (ii), (iii) and (iv).

77. The kit according to any one of claims 69-76, wherein the kit further comprises at least one additional agent useful for treating peanut allergy.

78. The kit according to any one of claims 69-77, wherein the kit further comprises a peanut allergen.

79. The antibody, or an antigen-binding fragment thereof, according to any one of claims 1 to 45, the nucleic acid or the plurality of nucleic acids according to any one of claims 48 to 51, the vector or the plurality of vectors according to claim 52 or 53, the cell according to claim 54, the composition according to any one of claims 56 to 68, or the kit according to any one of claims 69 to 78 for use as a medicament.

80. The antibody, or an antigen-binding fragment thereof, according to any one of claims 1 to 45, the nucleic acid or the plurality of nucleic acids according to any one of claims 48 to 51, the vector or the plurality of vectors according to claim 52 or 53, the cell according to claim 54, the composition according to any one of claims 56 to 68, or the kit according to any one of claims 69 to 78 for use in the prophylaxis or treatment of a peanut allergy.

81. The antibody, or the antigen-binding fragment thereof, the nucleic acid or the plurality of nucleic acids, the vector or the plurality of vectors, the cell, or the composition for use according to claim 80, wherein at least two, three or four distinct antibodies or antigen-binding fragments thereof, which bind to distinct non-overlapping epitopes of Ara h 2; or nucleic acid(s) or vector(s) encoding the distinct antibodies; or composition(s) comprising the distinct antibodies are administered.

82. The antibody, or the antigen-binding fragment thereof, the nucleic acid or the plurality of nucleic acids, the vector or the plurality of vectors, the cell, or the composition for use according to claim 80 or 81, wherein the administration of the antibody, or the antigen-binding fragment thereof, the nucleic acid or the plurality of nucleic acids, the vector or the plurality of vectors, the cell, or the composition is combined with the administration of a peanut allergen, preferably selected from the group consisting of Ara h1, Ara h2, Ara h3 and Ara h6 or a mixture thereof.

83. The antibody, or the antigen-binding fragment thereof, or the composition for use according to claim 82, wherein the antibody, or the antigen-binding fragment thereof, or the composition is administered before or during a desensitization procedure with a peanut allergen.

84. Use of the antibody, or an antigen-binding fragment thereof, according to any one of claims 1 to 45, the composition according to any one of claims 56 to 68, or the kit according to any one of claims 69 to 78 in (in-vitro) diagnosis of a peanut allergy.

85. Use of the antibody, or an antigen-binding fragment thereof, according to any one of claims 1 to 45, according to any one of claims 56 to 68, or the kit according to any one of claims 69 to 78 in a method for detecting a peanut allergen.

86. Use of the antibody, or an antigen-binding fragment thereof, according to any one of claims 1 to 45, the nucleic acid or the plurality of nucleic acids according to any one of claims 48 to 51, the vector or the plurality of vectors according to claim 52 or 53, the cell according to claim 54, the composition according to any one of claims 56 to 68, or the kit according to any one of claims 69 to 78 in the manufacture of a medicament for prophylaxis, treatment or attenuation of a peanut allergy.

87. A method of treating, ameliorating or reducing a peanut allergy, or lowering the risk of a peanut allergic or anaphylactic reaction, comprising: administering to a subject in need thereof, a therapeutically effective amount of the antibody, or an antigen-binding fragment thereof, according to any one of claims 1 to 45, the nucleic acid or the plurality of nucleic acids according to any one of claims 48 to 51, the vector or the plurality of vectors according to claim 52 or 53, the cell according to claim 54, the composition according to any one of claims 56 to 68, or the kit according to any one of claims 69 to 78.
